(19) United States
(12) Patent Application Publication
(10) Pub. No.: US 2009/0227658 A1
(43) Pub. Date: Sep. 10, 2009

(54) Methods and compositions for immunization against hiv

(76) Inventors: Yaoxing Huang, Brooklyn, NY (US); David D. Ho, Chappaqua, NY (US); Zhiwei Chen, New York, NY (US)

Correspondence Address: Thomas J. Kowalski;Frommer Lawrence & Haug, 745 Fifth Avenue, New York, NY, 10151, US

(21) Appl. No.: 10/570,512

(22) PCT Filed: Oct. 17, 2003
(86) PCT No.: PCT/US03/33112

§ 371 Date: Mar. 1, 2006

Related U.S. Application Data
(60) Provisional application No. 60/419,465, filed on Oct. 18, 2002.

(51) Int. Cl. A61K 31/7088; C12N 15/63

(52) U.S. Cl. 514/44 R; 435/320.1

(57) ABSTRACT

The present invention relates to nucleic acid and attenuated vaccinia vectors for prophylactic use against HIV infection, as well as methods of eliciting immune responses in subjects susceptible to HIV infection. The prophylactic vaccine regimen of the invention involves immunological priming with an inoculum comprising two novel DNA vectors, followed by boosting with a Modified Vaccinia Ankara (MVA) recombinant viral vector expressing the corresponding HIV proteins.

### CROSS-REFERENCE TO RELATED APPLICATIONS

This application claims priority to U.S. Provisional Application Ser. No. 60/419,465, filed on Oct. 18, 2002.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### STATEMENT OF RIGHTS TO INVENTIONS MADE UNDER FEDERALLY SPONSORED RESEARCH

The Aaron Diamond AIDS Research Center (ADARC) and International AIDS Vaccine Initiative (IAVI) provided funding for developments of inventions herein. ADARC and IAVI may have certain rights to the invention.

### FIELD OF THE INVENTION

The present invention relate to nucleic acid and attenuated vaccinia vectors for therapeutic and prophylactic use against HIV infection, as well as methods of eliciting immune responses in a subject susceptible to HIV infection. The therapeutic and prophylactic vaccine regimens of the invention involves immunological priming with an inoculum comprising two novel nucleic acid vectors, followed by boosting with a Modified Vaccinia Ankara (MVA) recombinant viral vector expressing the corresponding HIV proteins. Other aspects of the invention are described in or are obvious from the following disclosure, and are within the ambit of the invention.

### BACKGROUND OF THE INVENTION

Despite two decades of effort against it, the global HIV epidemic continues to plague humanity. In the face of such an unprecedented medical challenge, the scientific community has made important advances in the fields of virology, immunology and pharmacology. Nonetheless, it has proven extremely difficult both to contain the spread of infection around the world, and to prevent disease progression in most infected individuals. Since the beginning of the epidemic, 65 million people have been infected. Globally, over 42 million people are today living with HIV infection, with 5 million new infections acquired annually in 2002 (AIDS epidemic update, December 2002. Joint UNAIDS/WHO). More than 25 million individuals have lost their lives to the disease since the beginning of the pandemic; 3 million people died of AIDS in 2002 alone. Over 95% of new HIV infections occur in developing countries, with the majority of infections found in Sub-Saharan Africa and South East Asia. Of the 5% who have access to antiretroviral medication, a significant subset will be intolerant of available drugs because of adverse effects, and another subset will harbor drug-resistant viral variants. Though public health outreach can help slow the rate of HIV transmission in certain regions, it is clear that a protective vaccine would represent the most satisfying solution to the global problem.

Two broad classes of the HIV virus have been identified, HIV-1 and HIV-2. Three classes of HIV-1 have developed across the globe: M (major), O (outlying) and N (new). Among the M group, which accounts for >90% of reported HIV/AIDS cases, viral envelopes have diversified so greatly that this group has been subclassified into nine major clades including A-D, F-H, J and K, as well as several circulating recombinant forms. Viral diversity appears to radiate out of sub-Saharan Africa, where over 28 million of the total 40 million infected persons live. The other genre of retrovirus, HIV-2, has not spread much beyond West Africa, where it is presently endemic. Some sporadic cases have been observed elsewhere in Africa but the virus appears to be significantly less pathogenic than HIV-1.

One particular subtype of HIV-1 appears to have achieved phylogenetic dominance. Subtype C viruses now account for over 50% of new HIV-1 infections in the world. In particular, this clade has ravaged much of sub-Saharan Africa, and is now encroaching into Indochina (Beyrer, C. et al, (2000) AIDS, 14(1): 75-83; Yu, X. F., (2001) AIDS 15(4): 523-5; Piyasirisilp, S. et al, (2000) AIDS 74(23): 11286-95). Via India and Myanmar, subtype C has gained a foothold in the People's Republic of China, presumably through transmission among intravenous drug-users (IVDUs) in the southwestern part of the country. Yunnan province is especially burdened, with nearly half of the HIV-1 cases in all of China. According to the Yunnan Bureau of Health, the prevalence of HIV-1 infection among IVDUs in the province was 29% in 2000, and is expected to reach 40.7% in 2005. Five counties in Yunnan (Wenshan, Honghe, Dehong, Lingchang and Dali) have the highest prevalence rates, estimated at between 50 and 75%. HIV-1 subtype C has also spread to neighboring provinces, such as Sichuan and Guangxi, and is additionally responsible for much of the infection in distant Xinjiang.

DNA vaccination, or genetic immunization, is a promising new strategy in vaccinology. Cell-mediated immunity (CMI) is known to be critical for controlling HIV-1 replication (Ogg et al. (1998) Science 279: 2103-6; Schmitz et al. (1999) Science 283: 857-60; Jin et al. (1999) J. Exp. Med. 189:991-8; McMichael et al. (2001) Nature 410: 980-7). Early attempts to design vaccines against HIV-1 revealed that conventional approaches such as protein/subunit or inactivated virus are ineffective against retroviral infection. Perhaps as a consequence of the de novo expression involved, DNA vaccination appears to allow for better antigen presentation towards generation of CMI. In one study, DNA-vaccination resulted in at least partial protection of rhesus macaques from experimental challenge with pathogenic SHIV (Barouch et al. (2600) Science 290: 486-92). In combination with a recombinant vector as a prime-boost regimen, however, DNA vaccination proves even more effective at stimulating CMI and containing infection with SHIV (Robinson et al. (1999) Nat. Med. 5: 526-34; Hanke et al. (1999) Vaccine 17: 589-96; Hanke et al. (1999) J. Virol. 73: 7524-32; Allen et al. (2000) J. Immunol. 164: 4968-78; Amara et al. (2001) Science 292: 69-74; Barouch et al. (2001) J. Virol. 75: 5151-8).

The use of live attenuated vaccines designed using HIV itself is widely held to be unacceptably risky for use against the virus. Therefore, an effective vaccine regimen using nucleic acids, alone or in combination with an attenuated non-lentiviral boost, would provide a significant advance in a field where other vaccination strategies have thus far been unsuccessful.

### SUMMARY OF THE INVENTION

A therapeutic and prophylactic vaccine against HIV that is safe and effective has thus far been a major challenge. Traditional methods of vaccinology have proven to be ineffective or unsafe for use against HIV, however it has now been unexpectedly shown that a nucleic acid vaccine against HIV, administered either alone or in combination with an attenuated poxyiral boost, is effective in the priming of an immune response against selected HIV antigenic determinants. Therefore, the present invention relates to nucleic acid and attenuated vaccinia vectors for therapeutic and prophylactic use against HIV infection, as well as compositions and methods of eliciting immune responses in subjects susceptible to HIV infection. The therapeutic and prophylactic vaccine regimens of the invention involves immunological priming with an inoculum comprising two novel nucleic acid vectors, followed by boosting with a recombinant MVA expressing the corresponding HIV proteins.

Other aspects of the invention are described in or are obvious from the following disclosure, and are within the ambit of the invention.

A first aspect of the present invention provides nucleic acid vectors comprising at least one HIV sequence operably linked to a promoter and which encode a protein(s) that does not assemble into viral particles.

In another aspect, the nucleic acid vectors comprise at least two HIV sequences, each operably linked to separate promoters and which encode proteins that do not assemble into viral particles.

The HIV sequences described herein are selected from the group consisting of env, gag, pol, tat, rev, nef, vif, vpr, vpu, vpx, muteins, fusions, and portions thereof.

The promoters comprise heterologous promoters selected from the group consisting of prokaryotic promoters, eukaryotic promoters, and viral promoters. In an embodiment, the eukaryotic promoter is human eukaryotic initiation factor-1α promoter, and the viral promoter of the nucleic acid vector is the cytomegalovirus immediate/early promoter.

The present invention also describes use of transcriptional termination sequences positioned downstream of the HIV sequences in the nucleic acid vectors. The transcriptional terminators can be polyadenylation signals selected from the group consisting of the bovine growth hormone polyadenylation signal, the SV40 polyadenylation signal, and the vaccinia virus polyadenylation signal.

The invention further describes at least one HIV sequence operably linked to a heterologous leader sequence. The leader sequences can be the tissue plasminogen activator (tPA) leader sequence, but can also comprise the yeast α-factor mating pheromone leader sequence, the pre-pro-insulin leader sequence, and the invertase leader sequence, the immunoglobulin A leader sequence, and the ovalbumin leader sequence, among others.

The nucleic acid vectors of the invention comprise HIV Gag operably linked to the tPA leader sequence such that viral particles are not assembled. In an embodiment of the invention, the HIV sequences are selected from the group consisting of SEQ ID NO:7, SEQ ID NO:9, SEQ ID NO:11, SEQ ID NO:13; SEQ ID NO:17; and SEQ ID NO:19.

Origins of replication that direct propagation and amplification of the nucleic acid vectors in unicellular organisms are also contemplated in the nucleic acid vectors of the invention. These origins can be, but are not limited to, the colE1 (pMB1) origin, the 2μ yeast origin, eukaryotic centromeric regions, eukaryotic autonomously replicating sequences, the SV40 origin, and the cytomegalovirus (CMV) origin.

The nucleic acid vectors of the invention further comprise selectable marker genes, which can be antibiotic resistance genes. Such resistance genes can be attributed to the antibiotics ampicillin, tetracycline, kanamycin, doxycycline, neomycin, hygromycin, bleomycin, zeocin, puromycin, and chloramphenicol, among others.

In another aspect of the invention, nucleic acid vectors comprising at least two HIV sequences are provided, wherein the HIV sequences are each operably linked to separate promoters, encode proteins that do not assemble into viral particles. Further, at least one HIV sequence is operably linked to a heterologous leader sequence. The vector optionally further comprises a downstream transcriptional terminator, an origin of replication, and a selectable marker gene.

The nucleic acid vectors of the invention can be viral vectors, such as a modified vaccinia Ankara (MVA) vector, an ALVAC vector, a NYVAC. 1 vector, or a NYVAC.2 vector. Preferably, the viral vector is an MVA vector that comprises at least two HIV sequences inserted into deletion site III of the MVA genome, wherein each HIV sequence is operably linked to separate promoters and wherein the HIV sequences encode proteins that do not assemble into viral particles.

The poxyiral promoters used to express the HIV sequences are selected from the group consisting of the poxyiral 7.5K promoter, the poxyiral 40K promoter, the poxyiral H5 promoter, the poxyiral 11K promoter, the poxyiral I3 promoter, the poxyiral synthetic (SYN) promoter, and the poxyiral synthetic early/late promoter. In another embodiment, the promoters are different promoters.

Another aspect utilizes nucleic acid vectors that are MVA vectors that comprise at least two HIV sequences inserted into deletion site III of the MVA genome, at least one HIV sequence inserted into deletion site II of the MVA genome; and wherein each HIV sequence is operably linked to a separate promoter. Additionally, the HIV sequences encode proteins that do not assemble into viral particles.

The HIV sequences, described in this disclosure are selected from the group consisting of env, gag, pol, tat, rev, nef, vif, vpr, vpu, vpx, muteins, fusions, and portions thereof.

The promoters described in the viral vectors of the invention are selected from the group consisting of the poxyiral 7.5K promoter, the poxyiral 40K promoter, the poxyiral H5 promoter, the poxyiral 11K promoter, the poxyiral I3 promoter, the poxyiral synthetic (SYN) promoter, and the poxyiral synthetic early/late promoter. In another embodiment, the promoters are different promoters.

The HIV sequences of the invention further comprise heterologous leader sequences selected from the group consisting of the tPA leader sequence, yeast α-factor mating pheromone leader sequence, the pre-pro-insulin leader sequence, the invertase leader sequence, the immunoglobulin A leader sequence, the β-globin leader sequence, and the ovalbumin leader sequence.

In an embodiment of the invention, the HIV sequences SEQ ID NO:17 and SEQ ID NO:19 are inserted into deletion site III of MVA and SEQ ID NO:21 is inserted into deletion site II of MVA.

The invention also describes a nucleic acid vector comprising a viral vector selected from the group consisting of ALVAC, MVA, NYVAC.1 and NYVAC.2. One embodiment of the invention provides an MVA vector comprising tPA-delta V2 env and tPA-gag-pol inserted into deletion site III of MVA and tPA-nef-tat inserted into deletion site II of MVA.

Compositions comprising any of the nucleic acid vectors described herein are also envisioned in this disclosure. In one embodiment, each nucleic acid vector comprises different HIV sequences. Preferably, tPA-env and tPA-gag are on a first nucleic acid vector and tPA-nef-tat are on a second nucleic acid vector. Even more preferably, SEQ ID NO:7 and SEQ ID NO:9 are on a first nucleic acid vector and SEQ ID NO:11 AND SEQ ID NO:13 are on a second nucleic acid vector.

Another aspect of the invention describes a composition wherein tPA-env and tPA-gag are on a first nucleic acid vector, and tPA-pol and tPA-nef-tat are on a second nucleic acid vector. The composition further comprises tPA-delta V2 env and tPA-gag-pol inserted into deletion site III of MVA and tPA-nef-tat inserted into deletion site II of MVA. Preferably, SEQ-ID NO:7 and SEQ ID NO:9 are on a first nucleic acid vector and SEQ ID NO:11 and SEQ ID NO:13 are on a second nucleic acid vector. The composition further comprises SEQ ID NO:17 AND SEQ ID NO:19 inserted into deletion site II of MVA.

The present invention is additionally directed to pharmaceutical compositions comprising the nucleic acid vectors described above, in a pharmaceutically acceptable carrier, adjuvant, or excipient.

Additionally, the invention relates to methods of eliciting an immune response in a subject susceptible to an HIV-related disease or condition. The methods comprise administration of the nucleic acid vectors, compositions, and pharmaceutical compositions described in this disclosure to the subject, thereby eliciting an immune response against HIV.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1 is a schematic map of pVAX1.

FIG. 2 is a schematic map of pADVAX, a modified pVAX1 vector including the human elongation factor 1α (hEF1α) as a second promoter.

FIG. 3 is a bar graph showing expression of gag genes from transiently transfected 293T cells of native pVAX1, codon optimized pADVAX, and codon-optimized tPA pADVAX as measured using a commercially available ELISA kit that quantifies HIV Gag (p24).

FIG. 4 is a Western blot of env expression in 293T cells transfected DNA constructs as follows: native gp160 with rev (A), optimized gp160 with native signal peptide (B), and optimized gp160 with tPA signal peptide.

FIG. 5 is a Western blot showing expression of env and gag driven by the pADVAX dual promoter-vector. The vertical arrow indicates protein expression from ADVAX I.

FIG. 6 is a schematic representation of the modifications of the pol gene made for ADVAX II, wherein PR=protease, RT=reverse transcriptase, IN=integrase. The deletion in protease (DTGA) comprises amino acids 25-28 of the wild-type gene. The point mutation in reverse transcriptase (M to G) is position 184 of the wild-type gene. The Western blot was performed on cell lysates of 293T cells transfected with pVAX1-tPA-mutated pol (A) and pVAX1 alone (B). Uncleaved tPA-Pol is 110 kD.

FIG. 7 is a Western blot depicting protein expression from cell culture supernatants transfected with nef-tat.

FIG. 8 is a Western blot showing protein expression from cell culture supernatants transfected with tPA-nef-tat.

FIG. 9 shows the results of flow cytometric analysis of MHC-class I expression of 293T cells transfected with nef constructs: vector alone, nef, tPA-nef, and tPA-nef-tat.

FIG. 10 is a schematic map of ADVAX I and ADVAX II.

FIG. 11 shows IFN-γ ELISpot responses to Env and Gag derived peptide pools. Peptides were 20-mers overlapping by 10. Each pool contains 12 peptides, except for Gag A-I, which represents a specific 9-mer (AMGMLKDTI) (SEQ ID NO:2) previously identified as an antigen-specific CD8+ epitope in BALB/c mice. Env P1 comprises amino acids 24-144, Env P4 comprises amino acids 403-573, Gag P3 comprises amino acids 251-380, and Gag A-I comprises amino acids 217-225.

FIG. 12 shows the Env- and Gag-specific IFN-γ ELISpot responses in mice vaccinated with different doses of ADVAX I.

FIG. 13 shows ELISA analysis of mice vaccinated with different DNA vaccines intramuscularly at 0, 3, and 6 weeks, using serum anti-Gag antibodies.

FIG. 14 shows IFN-γ ELISpot responses to Pol, Tat, and Nef derived peptide pools.

FIG. 15 shows Pol- and Nef-Tat specific IFN-γ ELISpot responses in mice vaccinated with different doses of ADVAX II.

FIG. 16 is a Western blot depicting HIV-1 nef expression after the nef gene was introduced into different insertion sites of the MVA genome.

FIG. 17 shows 293T cells expressing DV2 Env mediated cell fusion with HOS cells carrying the primary receptor CD4 and the secondary receptor CCR5.

FIG. 18 is a genomic map of ADMVA.

FIG. 19 is a schematic map showing the construction of pZC1 and pZC3 from pLW7.

FIG. 20 shows expression of recombinant env-gag-pol MVA by immunostaining using an anti-Env antibody (left panel) and Western Blot analysis (right).

FIG. 21 is a schematic map showing construction of pZC22 from pLW22.

FIGS. 22 and 23 show expression of Env and Nef from recombinant ADMVA by double-immunostaining with anti-Env and anti-Nef antibodies.

FIG. 24 is a Western blot showing all five inserted genes in ADMVA.

FIG. 25 shows immunostaining of HIV-1 Env with 10³-10⁸ ADMVA.

FIG. 26 depicts ADMVA infection of human cells.

FIG. 27 is a graph depicting IFN-γ ELISpot responses to HIV-1 Env, Gag, Pol, Nef, and Tat derived peptides of peptide pools.

FIG. 28 depicts IFN-γ ELISpot responses in BALB/c mice to homologous subtype C Env, Gag, Pol, Nef and Tat derived peptides or peptide pools.

FIG. 29 shows IFNγ ELISpot responses in B6×B10 mice to homologous subtype C Env, Gag, Pol, Nef, and Tat derived peptides or peptide pools.

FIG. 30 shows Env-specific IFN-γ ELISpot responses in BALB/c mice vaccinated with different doses of ADMVA.

FIG. 31 is a graph depicting HIV-1 specific antibody responses in BALB/c mice against Gag and gp120 proteins.

FIG. 32 shows HIV-1 specific antibody responses in BALB/c mice against gp120.

FIG. 33 is a graph showing Env-specific IFN-γ ELISpot responses in BALB/c mice vaccinated with ADMVA via different routes of immunization.

FIG. 34 shows MVA-specific IFN-γ ELISpot responses in BALB/c mice vaccinated with different doses of MVA.

### DETAILED DESCRIPTION OF THE INVENTION

As used herein, the following terms are described by the following meanings.

The terms “disorder associated with HIV infection” or “HIV-1 related disease”, and the like, herein refer to a disease state marked by HIV infection. Such disorders associated with HIV infection include, but are not limited to AIDS, Kaposi's sarcoma, opportunistic infections such as those caused by Pneumocystis carinii and Mycobacterium tuberculosis; oral lesions including thrush, hairy leukoplakia, and aphthous ulcers; generalized lymphadenopathy; shingles; thrombocytopenia; aseptic meningitis; neurologic disease such as toxoplasmosis, cyrptococcosis, CMV infection, primary CNS lymphoma, and HIV-associated dementia; peripheral neuropathies; seizures; and myopathy.

A “subject” is a vertebrate, preferably a mammal, more preferably a human. Mammals include, but are not limited to, humans, farm animals, sport animals, and pets.

A subject “susceptible to” HIV infection or an HIV-associated condition or disease is a subject who belongs to a group whose risk of HIV infections is higher than the risk of the population as a whole.

The terms “immunogenic composition”, “immunological composition” and “vaccine” relate to an immunological composition containing the vector (or an expression product thereof) that elicits an immunological or immune response—local or systemic. The response can, but need not be protective. An immunogenic composition containing the inventive recombinant or vector (or an expression product thereof) likewise elicits a local or systemic immunological response that can, but need not, be protective. A vaccine composition elicits a local or systemic protective response. Accordingly, the terms “immunological composition” and “immunogenic composition” include a “vaccine composition” (as the two former terms can be protective compositions). The invention comprehends immunological, immunogenic or vaccine compositions.

The term “therapeutically effective dose” means a dose that produces the desired effects for which it is administered. The exact dose will depend on the purpose of the treatment, and will be ascertainable by one skilled in the art using known techniques (see, for example, Lieberman (1992) Pharmaceutical Dosage Forms Vol. 1-3; Lloyd (1999) The Art, Science and Technology of Pharmaceutical Compounding; and Pickar (1999) Dosage Calculations). In the case of a therapeutically effective amount of a DNA vaccine of the invention, the therapeutically effective amount will be an amount necessary to achieve any indicia of success in the treatment of HIV infection or AIDS in an individual, including any objective or subjective criteria such as HIV viral inhibition, diminishing of symptoms associated with HIV infection and AIDS, or improvement of a patient's physical or mental well-being.

A “vector” is a tool that allows or faciliates the transfer of an entity from one environment to another (See “The Development of Human Gene Therapy” T. Friedmann Ed., 1999 Cold Spring Harbor Press). For example, some vectors used in recombinant DNA techniques allow entities, such as a segment of DNA (such as a heterologous transgene) to be transferred into a target cell. Optionally, once within the target cell, the vector may then serve to maintain the transgene within the cell or may act as a unit of DNA replication. Examples of vectors used in recombinant DNA techniques include plasmids, chromosomes, artificial chromosomes or viruses. The vectors of the present invention may be delivered to a target site by a non-viral (plasmid) or a viral vector.

As used herein, an antigen or antigenic determinant, such as gene products of HIV, is “reactive” with an antibody raised against the antigen when there is a specific binding event/reaction between the antigen and the antibody.

The term “host cell” refers to one or more cells into which a recombinant DNA molecule is introduced. Host cells of the invention include, but need not be limited to, bacterial, yeast, animal, insect and plant cells. Host cells can be unicellular, or can be grown in tissue culture as liquid cultures, monolayers or the like. Host cells may also be derived directly or indirectly from tissues.

A host cell is “transformed” by a nucleic acid when the nucleic acid is translocated into the cell from the extracellular environment. Any method of transferring a nucleic acid into the cell may be used; the term, unless otherwise indicated herein, does not imply any particular method of delivering a nucleic acid into a cell, nor that any particular cell type is the subject of transfer. Another term used in the art is “transfect”. Non-viral delivery systems include but are not limited to DNA transfection methods. Here, transfection includes a process using a non-viral vector to deliver a gene to a target eukaryotic cell such as a mammalian cell. Typical transfection methods include direct DNA injection, electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), and combinations thereof.

An “expression control sequence” is a nucleic acid sequence that regulates gene expression (i.e., transcription, RNA formation and/or translation). Expression control sequences may vary depending, for example, on the chosen host cell or organism (e.g., between prokaryotic and eukaryotic hosts), the type of transcription unit (e.g., which RNA polymerase must recognize the sequences), the cell type in which the gene is normally expressed (and, in turn, the biological factors normally present in that cell type).

A “promoter” is one such expression control sequence, and, as used herein, refers to an array of nucleic acid sequences that control, regulate and/or direct transcription of downstream (3′) nucleic acid sequences. As used herein, a promoter includes necessary nucleic acid sequences near the start site of transcription, such as, in the case of a polymerase II type promoter, a TATA element.

The term “operably linked” refers to functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “recombinant” when used herein with reference to portions of a nucleic acid or protein, indicates that the nucleic acid comprises 2 or more sub-sequences that are not found in the same relationship to each other in nature. For instance, a nucleic acid that is recombinantly produced typically has 2 or more sequences from distinct genes or non-adjacent regions of the same gene, synthetically arranged to make a new nucleic acid sequence encoding a new protein. The term “recombination” as used herein, refers to the process of producing a recombinant protein or nucleic acid by standard techniques known to those skilled in the art, and described in, for example, Sambrook et al., Molecular Cloning; A Laboratory Manual 2d ed. (1989).

The term “heterologous” in the context of the instant application refers to an element, such as a component of a plasmid vector (e.g. promoter, leader sequence) that is not normally associated with the nucleic acid molecule to which it is operably linked.

In this disclosure, “comprises,” “comprising,” “containing” and “having” and the like can have the meaning ascribed to them in U.S. Patent law and can mean includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

A first aspect of the present invention provides nucleic acid vectors comprising at least one HIV sequence operably linked to a promoter and which encode a protein(s) that does not assemble into viral particles.

In another aspect, the nucleic acid vectors comprise at least two HIV sequences, each operably linked to separate promoters and which encode proteins that do not assemble into viral particles.

The HIV sequences described herein are selected from the group consisting of env, gag, pol, tat, rev, nef, vif, vpr, vpu, vpx, muteins, fusions, and portions thereof.

The promoters comprise heterologous promoters selected from the group consisting of prokaryotic promoters, eukaryotic promoters, and viral promoters. In an embodiment, the eukaryotic promoter is human eukaryotic initiation factor-1α promoter, and the viral promoter of the nucleic acid vector is the cytomegalovirus immediate/early promoter.

The present invention also describes use of transcriptional termination sequences positioned downstream of the HIV sequences in the nucleic acid vectors. The transcriptional terminators can be polyadenylation signals selected from the group consisting of the bovine growth hormone polyadenylation signal, the SV40 polyadenylation signal, and the vaccinia virus polyadenylation signal.

The invention further describes at least one HIV sequence operably linked to a heterologous leader sequence. The leader sequence(s) can be the tissue plasminogen activator (tPA) leader sequence, but can also comprise the yeast α-factor mating pheromone leader sequence, the pre-pro-insulin leader sequence, and the invertase leader sequence, the immunoglobulin A leader sequence, and the ovalbumin leader sequence, among others.

The nucleic acid vectors of the invention comprise HIV Gag operably linked to the tPA leader sequence such that viral particles are not assembled. In an embodiment of the invention, the HIV sequences are selected from the group consisting of sequences provided herein, SEQ ID NO:7, SEQ ID NO:9, SEQ ID NO:11, SEQ ID NO:13; SEQ ID NO:17; and SEQ ID NO:19.

Origins of replication to direct propagation and amplification of the nucleic acid vectors in unicellular organisms are also contemplated in the nucleic acid vectors of the invention. These origins can be, but are not limited to, the colE1 (pMB1) origin, the 21 yeast origin, eukaryotic centromeric regions, eukaryotic autonomously replicating sequences, the SV40 origin, and the cytomegalovirus (CMV) origin.

The nucleic acid vectors of the invention further comprise selectable marker genes, which can be antibiotic resistance genes. Such resistance genes can be attributed to the antibiotics ampicillin, tetracycline, kanamycin, doxycycline, neomycin, hygromycin, bleomycin, zeocin, puromycin, chloramphenicol, among others.

In another aspect of the invention, nucleic acid vectors comprising at least two HIV sequences are provided, wherein the HIV sequences are each operably linked to separate promoters, encode proteins that do not assemble into viral particles. Further, at least one HIV sequence is operably linked to a heterologous leader sequence. The vector optionally further comprises a downstream transcriptional terminator, an origin of replication, and a selectable marker gene.

The nucleic acid vectors of the invention can be viral vectors, such as a modified vaccinia Ankara (MVA) vector, an ALVAC vector, a NYVAC. 1 vector, or a NYVAC.2 vector. Preferably, the viral vector is an MVA vector that comprises at least two HIV sequences inserted into deletion site III of the MVA genome, wherein each HIV sequence is operably linked to separate promoters and wherein the HIV sequences encode proteins that do not assemble into viral particles.

The poxyiral promoters used to express the HIV sequences are selected from the group consisting of the poxyiral 7.5K promoter, the poxyiral 40K promoter, the poxyiral H5 promoter, the poxyiral 11K promoter, the poxyiral I3 promoter, the poxyiral synthetic (SYN) promoter, and the poxyiral synthetic early/late promoter. In another embodiment, the promoters are different promoters.

Another aspect utilizes nucleic acid vectors that are MVA vectors that comprise at least two HIV sequences inserted into deletion site III of the MVA genome, at least one HIV sequence inserted into deletion site II of the MVA genome, and wherein each HIV sequence is operably linked to a separate promoter. Additionally, the HIV sequences encode proteins that do not assemble into viral particles.

The HIV sequences described in this disclosure are selected from the group consisting of env, gag, pol, tat, rev, nef, vif, vpr, vpu, vpx, muteins, fusions, and portions thereof.

The promoters described in the viral vectors of the invention are selected from the group consisting of the poxyiral 7.5K promoter, the poxyiral 40K promoter, the poxyiral H5 promoter, the poxyiral 11K promoter, the poxyiral I3 promoter, the poxyiral synthetic (SYN) promoter, and the poxyiral synthetic early/late promoter. In another embodiment, the promoters are different promoters.

The HIV sequences of the invention further comprise heterologous leader sequences selected from the group consisting of the tPA leader sequence, yeast α-factor mating pheromone leader sequence, the pre-pro-insulin leader sequence, the invertase leader sequence, the immunoglobulin A leader sequence, the β-globin leader sequence, and the ovalbumin leader sequence.

In an embodiment of the invention, the HIV sequences SEQ ID NO:17 and SEQ ID NO:19 are inserted into deletion site III of MVA and SEQ ID NO:21 is inserted into deletion site II of MVA.

The invention also describes a nucleic acid vector comprising a viral vector selected from the group consisting of ALVAC, MVA, NYVAC.1 and NYVAC.2. One embodiment of the invention provides an MVA vector comprising tPA-delta V2 env and tPA-gag-pol inserted into deletion site III of MVA and tPA-nef-tat inserted into deletion site II of MVA.

Compositions comprising any of the nucleic acid vectors described herein are also envisioned in this disclosure. In one embodiment, each nucleic acid vector comprises different HIV sequences. Preferably, tPA-env and tPA-gag are on a first nucleic acid vector and tPA-nef-tat are on a second nucleic acid vector. Even more preferably, SEQ ID NO:7 and SEQ ID NO:9 are on a first nucleic acid vector and SEQ ID NO:11 AND SEQ ID NO:13 are on a second nucleic acid vector.

Another aspect of the invention describes a composition wherein tPA-env and tPA-gag are on a first nucleic acid vector, and tPA-pol and tPA-nef-tat are on a second nucleic acid vector. The composition further comprises tPA-delta V2 env and tPA-gag-pol inserted into deletion site III of MVA, and tPA-nef-tat inserted into deletion site II of MVA. Preferably, SEQ ID NO:7 and SEQ ID NO:9 are on a first nucleic acid vector and SEQ ID NO:11 and SEQ ID NO:13 are on a second nucleic acid vector. The composition further comprises SEQ ID NO:17 and SEQ ID NO:19 inserted into deletion site II of MVA.

The present invention is additionally directed to pharmaceutical compositions comprising the nucleic acid vectors described above, in a pharmaceutically acceptable carrier, adjuvant, or excipient.

Additionally, the invention relates to methods of eliciting an immune response in a subject susceptible to an HIV-related disease or condition. The methods comprise administration of the nucleic acid vectors, compositions, and pharmaceutical compositions described in this disclosure to the subject, thereby eliciting an immune response against HIV.

A wide variety of nucleic acid vectors may be employed in housing the HIV nucleic acid sequences used in the compositions and vaccines of this invention. It will be apparent to one skilled in the art that nucleic acid vectors of the invention must have the capability to be produced at high volume, yet at the same time, must be capable of being expressed in a host of interest. Therefore, nucleic acid vectors can contain sequences that will allow their expression and amplification in unicellular hosts such as bacteria or yeast. Useful expression vectors include, but are not limited to, pVAX1, pGEM, pSP72, pcDNA, and other commercially available cloning vehicles.

Nucleic acids designed as a vaccine composition and that have been amplified in bacteria must undergo extensive purification in order to remove bacterial cell wall components that can cause infection, inflammation, and disease. These “endotoxins” are also called “lipopolysaccharides”, or “LPS”. Endotoxins can be removed by filtration methods well known in the art. An alternative method of plasmid vector amplification is the use of yeasts, such as Saccharomyces cerevisiae, among others.

In addition, any of a wide variety of expression control sequences, also heretofore used interchangeably with the analogous term “promoter”, may be used in the nucleic acid vectors to express the HIV sequences used in the compositions and methods of this invention. In selecting an expression control sequence, a variety of factors should also be considered. These include, for example, the relative strength of the promoter sequence, its controllability, and its compatibility with the DNA sequence of the pepfides described in this invention, in particular with regard to potential secondary structures. Such useful expression control sequences include heterologous expression control sequences such as prokaryotic promoters, eukaryotic promoters, and viral promoters.

Examples of useful viral promoters include, for example, the early and late promoters of SV40, cytomegalovirus, bovine papilloma virus, cytomegalovirus, retroviruses including lentiviruses, adeno-associated virus, and adenovirus, the T3 and T7 promoters, the major operator and promoter regions of phage lambda, the control regions of fd coat protein. Prokaryotic promoters such as, but not limited to, the lac system, the trp system, the TAC or TRC system, can also be used. Eukaryotic promoters that can be used advantageously to express the HIV sequences in the nucleic acid vectors of the invention include, but are not limited to, the human eukaryotic initiation factor 1 promoter, the promoter for 3-phosphoglycerate kinase, alcohol dehydrogenase, pyruvate kinase, or other glycolytic enzymes, the promoters of acid phosphatase, e.g., Pho5, α- and β-actin, and other constitutive and inducible promoter sequences known to control the expression of genes of prokaryotic or eukaryotic cells or their viruses, and various combinations thereof.

It is understood that not all vectors and promoters will function equally well to express the HIV sequences in the nucleic acids and compositions mentioned herein. However, one of skill in the art may make a selection among these vectors, promoters, and hosts without undue experimentation and without departing from the scope of this invention. For example, in selecting a vector, the host must be considered because the vector must be replicated in it. The vector's copy number, the ability to control that copy number, the ability to control integration, the presence and use of enhancer sequences, if any, and the expression of any other proteins encoded by the vector, such as antibiotic or other selection markers, should also be considered.

Optimal expression of HIV sequences also benefit from addition of transcriptional termination sequences at the 3′ end of the inserted sequences. Transcriptional terminators vary widely between organisms and can comprise nucleic acid sequences that advantageously form secondary structures in vivo, such as stem-loop structures. In this case, termination depends on the RNA product and is not determined simply by scrutiny of the DNA sequence during transcription. Well-known prokaryotic termination mechanisms include intrinsic termination, wherein the RNA polymerase core enzyme can terminate at certain sites in the absence of any other factor. Another well-characterized termination mechanism is dependent on the prokaryotic Rho factor, a 46 kD protein that is intimately involved in disengaging RNA polymerase from the cognate RNA strand. Other transcriptional terminators are well known in the art and include polyadenylation signals such as the bovine growth hormone polyadenylation signal and viral polyadenylation signals, such as those from vaccinia and the simian virus 40. Transcription termination often occurs at sites considerably downstream of the sites that, after polyadenylation, are the 3′ ends of most eukaryotic mRNAs. Polyadenylation is the non-templated addition of a 50 to 200 nt chain of polyadenylic acid (polyA). Cleavage must precede polyadenylation. These polyadenylation signals often comprise the sequence AAUAAA, which when deleted or mutated, prevents generation of polyadenylated mRNA.

Leader sequences are defined as sequences at the end of either nucleic acids (DNA and RNA) or proteins that must be processed off to allow for a specific function of the mature molecule. Leader sequences direct placement of proteins in a specific cellular compartment. An analogous term for leader sequence is “signal sequence”. For ER resident proteins and proteins destined for the lysosome or peroxisome, the signal sequence directs their return to their respective cellular compartments. Membrane proteins and proteins destined for secretion also require signal sequences, wherein the proteins are directed into specific secretion pathways destined for the plasma membrane, extracellular space, or via endosomal sorting. Signal sequences show no conservation of sequence. Signal sequences generally begin within 10 amino acids of the N-terminus. They are between 20 and 30 residues in length, characterized by a central hydrophobic core of approximately 10 to 15 (but no less than 6) residues, with a marked preference for leucines or alanines. They are flanked on the N-terminal side by a positively charged stretch of polar residues and by a neutral, but polar, C-terminal region. They are remarkably tolerant of amino acid substitutions, as long as their central hydrophobic character is retained. In an embodiment of the invention, the tissue plasminogen activator is used advantageously to direct translated HIV sequences into a secretory pathway that differs from the native HIV proteins, such that viral particles are not produced. Other examples of leader sequences include the yeast α-factor mating pheromone leader sequence, the pre-pro-insulin leader sequence, the invertase leader sequence, the immunoglobulin A leader sequence, the β-globin leader sequence, and the ovalbumin leader sequence.

Selectable markers can be used to assay for the presence of the nucleic acid vector in host or host cells of interest. Commonly used selectable markers include genes that when expressed, result in antibiotic resistance in the host. Such genes confer resistance to numerous antibiotics, such as, but not limited to, ampicillin, tetracycline, doxycycline, kanamycin, neomycin, bleomycin, puromycin, zeocin, hygromycin, and chloramphenicol. Reporter genes can also be used to monitor expression of the vector, such as the lacZ gene product, however these are not recommended, as reporter genes commonly used in the art encode for foreign proteins that can stimulate unwanted or unforeseen immune responses when administered in the subject.

Similarly, under certain circumstances wherein antibiotic resistance may be undesirable, such as when the goal is to generate a pure biological product in high yield for administration in a clinical setting, the use of antibiotics can present three main problems. First, a loss of selective pressure during intensive culture conditions (e.g. high biomass or continuous culture) can lead to antibiotic degradation or inactivation, resulting in product yield reduction. Secondly, the product is inevitably contaminated with the residual antibiotic, which in some cases, increases risk of immune sensitization and even anaphylaxis in the subject. Finally, there is also a risk of the spread of drug resistance after gene transfer to environmental organisms and in particular, pathogens. A repressor titration system, the methods of which are incorporated by reference (Williams, S. G. et al, (1998) Nucleic Acids Res. 26(9): 2120-2124; U.S. Pat. No. 5,972,708), can be advantageously used to amplify nucleic acid products in prokaryotic hosts without the presence of an antibiotic resistance gene.

Instead, selection of cells containing the plasmid occurs by using a molar excess of plasmid over chromosomal genomes to competitively titrate a repressor from a host selectable gene. In other words, the system uses the plasmid molecule itself to activate selection. It requires 1) that the host strain contains a chromosomal gene encoding a product essential to cell survival or growth, 2) that the gene is negatively regulated by a repressor protein such as the λ repressor, 3) an intracellular repressor concentration just sufficient to achieve repression of the gene, 4) that the plasmid contains a binding site for the repressor, and 5) that the plasmid copy number per cell is sufficient to achieve repressor titration.

Origins of replication are defined as the position on the DNA at which replication start points are found. In the context of plasmid-borne origin sequences, an origin is a DNA sequence that, when added to a non-replicating DNA causes it to replicate. Origins may also be described as a DNA sequence that in vitro is the binding target for enzyme complexes known to function in initiation of DNA replication. Commonly used origins are the ColE1 (pMB1) origin, the yeast 2μ origin, eukaryotic autonomously replicating sequences (ARS), eukaryotic centromeric sequences, the SV40 origin, the CMV origin, among others.

Viral delivery systems include but are not limited to adenovirus vector, adeno-associated viral (AAV) vector, a herpes viral vector, retroviral vector, or lentiviral vector. Other examples of vectors include ex vivo delivery systems, which include but are not limited to DNA transfection methods such as electroporation, DNA biolistics, lipid-mediated transfection, compacted DNA-mediated transfection. In a preferred embodiment, poxyiral vectors are used to deliver nucleic acids. More preferably, the poxyiral vector used is a modified vaccinia Ankara viral vector, discussed further below.

Vaccinia virus now serves as a unique live vector for expressing genes within the cytoplasm of mammalian cells (Hu, S. L., et al. (1986) Nature 320: 537-40; Moss, B., et al. (1996) Adv. Exp. Med. Biol. 397: 7-13; Sutter, G., and B. Moss. (1992) Proc. Natl. Acad. Sci. USA 89: 10847-51). The vaccinia virus has been well described in the art and can be found in U.S. Pat. Nos. 6,340,462; 5,972,597; 5,942,235; 5,225,336; 5,204,243; 5,155,020; 5,110,587; 4,769,330; 4,722,848; and 4,603,112. As a scientific tool, recombinant vaccinia viruses have been used to investigate the types of immune response needed for protection against specific infectious diseases including AIDS (Girard, M. (1990) Cancer Detect. Prev. 14: 411-3; Haynes, B. F. (1996) Lancet 348: 933-7; Moss, B. (1996) Proc. Natl. Acad. Sci. USA 93:11341-8). Since vaccinia virus is infectious in humans, the major concern of using live vaccinia vectors is their safety. Conventional vaccinia viruses cannot be used in immunocompromised patients, such as those with HIV, hematologic malignancies or those undergoing treatment with chemotherapy (Mayr, A., and K. Danner. (1978) Dev Biol Stand 41:225-34). For this reason, several highly attenuated vaccinia virus strains have been developed for their use as smallpox vaccines (Paoletti (1996) Proc. Natl. Acad. Sci. USA 93:11349-53) (Moss, B., et al. (1996) Adv. Exp. Med. Biol. 397: 7-13; Sutter, G., and B. Moss. (1992) Proc. Natl. Acad. Sci. USA 89: 10847-51; Blanchard, T. J., et al. (1998) J Gen Virol 79: 1159-67; Paoletti, E. (1996) Proc Natl Acad Sci USA 93:11349-53). Although these attenuated viruses are no longer required for immunization against smallpox, their early use in humans has provided critical safety information to guide the selection of a proper strain for AIDS vaccine development.

Three highly attenuated and efficacious poxvirus-based vectors, including NYVAC (U.S. Pat. Nos. 6,596,279; 5,762,938; 5,494,807; 5,453,364; 5,378,457; 5,364,773, Canarypox (ALVAC; U.S. Pat. Nos. 5,863,542; 5,766,598; 5,756,103), and Modified Vaccinia Ankara (MVA), are available for targeted applications as recombinant vaccines in both human and veterinary medicine (Moss et al. (1996) Adv. Exp. Med. Biol. 397:7-13). Use of MVA is described in U.S. Pat. No. 5,185,146. Preferably, an orthopoxvirus or avipoxvirus that is host range-restricted and can replicate only in baby hamster kidney cells (BHK) and chicken embryonic fibroblasts (CEF) are advantageously used. U.S. Pat. No. 5,494,807 discloses the differences between ALVAC and NYVAC with respect to their abilities to replicate in specific hosts. MVA in particular has been used in large vaccine trials and clinical practice for primary vaccination of over 120,000 humans. No side effects have been associated with its use, even when high-risk patients received primary vaccination (Mayr et al. (1978) ZBL Bakt Hyg. I Abt. Orig. B 167: 375-90). MVA is a host-range-restricted vaccinia virus strain (Sutter, G., and B. Moss. (1995) Dev Biol Stand 84: 195-200; Wyatt, L. S., et al. (1998) Virology 251: 334-42). The MVA strain has been passaged over 570 times in chicken embryo fibroblasts (CEF) and has lost its ability to multiply in most mammalian cell lines because its genome contains six major deletions relative to the WR vaccinia strain (Altenburger et al. (1989) Arch. Virol. 105:15-27; Meyer et al. (1991) J. Gen. Virol, 71-1031-8; Mayr, A., (1978) Zentralbl Bakteriol [B] 167: 375-90; Meyer, H., et al. (1991) J Gen Virol 72: 1031-8; Stickl, H., et al. (1974) Dtsch Med Wochenschr 99: 2386-92). These deletions are located near both ends of the viral genome. Notably, one deletion affects a 55K as well as a 32K human host range gene. Further analysis has revealed that the deletions of about two thirds of host range genes are partially responsible for the viral attenuation. The MVA strain grows well in avian cells but is unable to multiply in human and most other mammalian cells tested. Nevertheless, the replication of MVA DNA appears normal, and both early and late viral proteins are synthesized in human cells (Sutter et al. (1992) Proc. Natl. Acad. Sci. USA 89:10847-51; Sutter, G., and, B. Moss. (1992) Proc Natl Acad Sci USA 89: 10847-51; Sutter, G., et al. (1994). Vaccine 12: 1032-40). Since recombinant gene expression is unimpaired in non-permissive human cells, MVA serves as a highly efficient and exceptionally safe vector (Moss, B., et al. (1996) Adv Exp Med Biol 397: 7-13). Importantly, the MVA strain has been used in large vaccine trials and clinical practice for primary vaccination of over 120,000 humans in the battle against smallpox. No side effects have been associated with its use, even when immune-suppressed macaques or patients received primary vaccination (Mayr, A., et al. (1978) Zentralbl Bakteriol [B] 167: 375-90; Hochstein-Mintzel, V., et al. (1972) Z Immunitatsforsch Exp Klin Immunol 144: 104-56; Stittelaar, K. J., et al. (2001) Vaccine 19: 3700-9).

The immunogenicity and the protective efficacy of the highly attenuated and replication-defective recombinant MVA have been demonstrated to be stronger than those of many conventional vaccinia viruses (VV). Using a multiplicity of infection (MOI)≧1, the highly attenuated strain MVA was the only representative of VV that induced significant amounts of IFN α/β, which are responsible for the antiviral effect. Replicable virus from five well-known conventional VV strains as well as the Chinese VV strain Tien Tan (VVTT) used as a recombinant vaccine failed to induce leukocyte IFN (IFNα; Buttner, M. et al. (1995) Vet. Immunol. 46: 237-50). In small animals, recombinant MVA strains expressing HA-NP genes not only induced serum IgG antibodies, mucosal IgA antibodies and strong CTL responses but also protected the lung infection of immunized mice from challenge with Influenza virus, even after oral immunization (Bender, B. S. et al. (1996) J. Virol. 70: 6418-24). Most importantly, macaques immunized with SIV/SHIV recombinant MVA more likely became long-term non-progressors than those immunized with an SIV recombinant NYCBH-VV (Hirsch, V. M. et al. (1996) 70:3741-52; Amara, R. R. et al. (2001) Science 292: 69-74). These macaques, just like HIV-1 infected human long-term nonprogressors, had low levels of primary plasma viremia followed by a sustained restriction of virus replication, which were associated with the maintenance of normal lymphocyte subsets and intact lymphoid architecture. These results, together with previous data on the safety of MVA in humans, suggest the potential usefulness of recombinant MVA for prophylactic vaccination, of AIDS in humans. At present, no HIV recombinant MVA has been constructed or used for HIV-1 vaccination in humans.

The multigenic recombinant ADMVA is designed as a homologous booster, corresponding to the plasmid DNA priming vaccine for HIV-1 subtype C. The HIV-1 structural genes (env, gag, pol) and regulatory genes (nef tat) are encoded in the construct. The genes used in our vaccines were derived from a clade C strain, (Circulating Recombinant Form 007, or HIV_{CHN.AD}, which also contains segments of clade B) that is the dominant subtype in Yunnan Province. Other HIV clades can also be advantageously substituted in the nucleic acid and viral vectors of the invention, without undue experimentation.

Vaccinia promoters are necessary when using vaccinia virus as a vector for gene transfer or gene expression, as the virus replicates in the cytoplasm of infected cells. Because of this unique feature, the virus encodes its own replication and transcription machinery, which specifically recognizes vaccinia promoters. This is in contrast with other viruses, which utilize the host's own mechanisms to carry out replication, transcription, and other processes for viral propagation. Promoters that can be advantageously used in the MVA vector include, but are not limited to, the 7.5K promoter, the 11K promoter, the 40K promoter, the H5 promoter, the I3 promoter, the SYN (synthetic) promoter, and the synthetic early/late promoter (sE/L) (Moss, et al, Biotechniques).

MVA achieved attenuation by over 570 serial passages in chicken embryonic fibroblasts. Analysis of the MVA genome revealed that attenuation might be attributed to loss of large fragments, mostly in regions of genes that were thought to be non-essential. There are six major naturally-occurring deletions in MVA and are denoted as deletions I through VI. These deletion sites presumably contained genes that were non-essential, and thus provide sites for insertion of heterologous genes. Use of any one of deletion sites I through VI can result in efficient and robust expression of genes of interest. In addition, the nonessential thymidine kinase gene also provides another site by which foreign genes may be inserted. Preferred embodiments use deletion site III. Deletion site II can also be used, separately or together with deletion site III.

Similarly, other attenuated poxviruses have regions of the genome that contain engineered deletions, which may or may not be essential. U.S. Pat. No. 5,766,882 describes a poxvirus that is defective such that it lacks a function imparted by an essential region of its parental poxvirus. The attenuated NYVAC vector describes similar regions of the genome, in which the thymidine kinase gene, the hemorrhagic region, the A type inclusion body region, the hemagglutinin gene, the host range gene region, and the large subunit, ribonucleotide reductase have been deleted therefrom (U.S. Pat. No. 5,364,773). Further, the NYVAC vector can additionally comprise deletion of the gene conferring interferon, thereby increasing safety in the host of interest.

There are two primary methods of producing recombinant MVA, homologous recombination and in vitro ligation. Homologous recombination is the original and still most widely used method of producing recombinant MVA (See U.S. Pat. No. 4,769,330). Cells are transfected with a transfer plasmid, which contains the recombinant gene under control of a vaccinia promoter flanked by several hundred base pairs of vaccinia-derived DNA, and infected with the virus. Recombination occurs between homologous sequences in the transfer plasmid and viral genome. A variety of methods are available for the isolation of recombinant MVA, including selection based on bromodeoxyuridine, or antibiotic resistance, detection of reporter gene expressing a calorimetric marker, complementation of a host range or small plaque phenotype (see, for example, U.S. Pat. No. 5,155,020), and direct antibody staining of plaques or DNA hybridization. The stable integration of a selectable marker precludes its use for selection of a second gene; in addition, extra genetic material may not be desirable in a recombinant MVA that is to be used in a subject. Schemes in which antibiotic-resistance or color marker genes are integrated and then spontaneously deleted by recombination have been developed, involving multiple rounds of plaque purification (Chakrabarti, S. et al. (1985) Mol. Cell. Biol. 5(12): 3403-9).

The in vitro ligation of a foreign gene into the MVA genome also provides an alternative to homologous recombination (see U.S. Pat. Nos. 6,265,183; 5,866,383; 5,445,953). Since MVA DNA is not infectious, the cells are transfected with MVA DNA and infected with a host-restricted helper virus, conditionally lethal virus, or otherwise defective virus (U.S. Pat. No. 5,204,243). These techniques allow efficient insertion of very large DNA fragments or even libraries of DNA fragments directly into the vaccinia genome. Recombinant VV genomes have been constructed with promoters and unique restriction sites to facilitate cloning and expression (Pfleiderer, M. et al (1995) J. Gen. Virol. 76(Pt.12): 2957-62; Merchlinsky, M. and B. Moss (1992) Virology 190(1): 522-6).

Human immunodeficiency virus is a retrovirus, of which there are many. Some examples of retroviruses include, but are not limited to: murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses. A detailed list of retroviruses may be found in Coffin et al. (“Retroviruses” 1997 Cold Spring Harbour Laboratory Press Eds: J M Coffin, S M Hughes, H E Varmus pp 758-763).

Retroviruses are broadly divided into two categories, namely “simple” and “complex”. Retroviruses are further sub-divided into seven groups. Five of these groups represent oncogenic retroviruses. The remaining two groups are the lentiviruses and the spumaviruses. A review of these retroviruses is presented in Coffin et al., 1997 (ibid). A distinction between the lentivirus family and other types of retroviruses is that lentiviruses have the capability to infect both dividing and non-dividing cells (Lewis, P. et al. (1992) EMBO J. 11: 3053-3058; Lewis, P. F. and M. Emerman (1994) J. Virol. 68: 510-516). In contrast, other retroviruses, such as MLV, are unable to infect non-dividing cells such as those that make up, for example, muscle, brain, lung and liver tissue. HUV falls under the category of “lentivirus”. In the context of this application, other lentivirus sequences may be advantageously used, such as FIV, SIV, EIAV, and the like.

Details on the genomic structure of some lentiviruses may be found in the art. Details on the HIV genome may be found in the NCBI Genbank database (i.e. Genome Accession Nos. AF033819; SEQ ID NOS:23-41). The HIV retroviral genome comprises genes called gag, pol and env, which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses. For the viral genome, the site of transcription initiation is at the boundary between U3 and R in the left hand side LTR and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves; gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction leads ultimately to infection by fusion of the viral membrane with the cell membrane.

Co-expression of gag, pol, and env result information of infectious virion particles. For the purposes of immunogenic compositions and vaccine production, formation of infectious particles would result in a dangerous and unacceptably risky situation. Mutations in the respective genes, such as in specific regions of pol, cause inactivation of viral infectivity, however, formation of virion particles can still occur. The present invention provides for expression of HIV sequences wherein the sequences encode proteins that do not assemble into infectious or non-infectious particles. Protein expression for the purposes of this disclosure serves to induce an immunological response. Activity of the protein, or even the presence of the full-length protein, is often not necessary to mount an immune response in a subject in need thereof. Plasmids comprising HIV sequences have been previously described. U.S. Pat. No. 5,665,577 describes HIV sequences that are expressed in plasmid vectors, but which encode proteins that form virions that do not contain sufficient HIV RNA to result in a replication competent HIV virion. U.S. Pat. No. 6,451,304 describes methods for producing replication-incompetent retrovirus vectors comprising transfecting cells with a first provirus plasmid that encodes gag, but not pol or envelope proteins; a second provirus plasmid that encodes pol, but not gag or envelope proteins; and a third separate envelope protein encoding construct.

HIV also contains additional genes that code for proteins other than gag, pol and env. Additional genes in HIV are vif vpr, vpx, vpu, tat, rev and nef Proteins encoded by additional genes serve various functions, some of which may be duplicative of a function provided by a cellular protein. In HIV, tat acts as a transcriptional activator of the viral LTR. It binds to a stable, stem-loop RNA secondary structure referred to as TAR. Rev regulates and co-ordinates the expression of viral genes through rev-response elements (RRE).

The predominant subtype that is found in the developed Western World, clade B, differs considerably from those subtypes and recombinants that exist in Africa and Asia, where the vast majority of HIV-infected persons reside. Thus, serious discrepancies may exist between the subtype B retrovirus that medical practitioners encounter in North America and Europe and those viral subtypes that plague humanity on a global scale (Spira, S. et al (2003) J. Antimicrob. Chemother. 51(2): 229-40). The large genomic diversity of viral subtypes in different geographical regions is the consequence of the astonishingly high mismatch error rate of the HIV reverse transcriptase (RT) enzyme coupled with the absence of an exonuclease proofreading activity. Other factors that contribute to the rapid pace of genetic diversification include the replicative rate of each viral subtype, the number of mutations arising in each replicative cycle, the viral propensity for genomic recombination and viral fitness. In addition, high rates of genomic evolution may result from host, environment and/or therapeutic selection pressures.

Three classes of HIV-1 have developed across the globe: M (major), 0 (outlying) and N (new). Among the M group, which accounts for >90% of reported HIV/AIDS cases, viral envelopes have diversified so greatly that this group has been subclassified into nine major clades including A-D, F-H, J and K, as well as several circulating recombinant forms. Viral diversity appears to radiate out of sub-Saharan Africa, where over 28 million of the total 40 million infected persons live.

A and A/G recombinant variants predominate in West and Central Africa. B has been the predominant species in Europe and the Americas. However, with increasing immigration and globalization, >40% of new infections in Europe are presently non-B African and Asian variants. C is largely predominant in Southern and Eastern Africa, India and Nepal. Indeed, clade C has created the recent epicentres of the HIV pandemic by its uncontrolled spread throughout Botswana, Zimbabwe, Malawi, Zambia, Namibia, Lesotho, South Africa, India, Nepal and China. D is generally limited to East and Central Africa, with sporadic cases observed in Southern and Western Africa. E has never materialized alone, but rather appears as an A/E mosaic detected in Thailand, the Philippines, China and Central Africa. F has been reported in Central Africa, South America and Eastern Europe. G and A/G recombinant viruses have been observed in Western and Eastern Africa as well as in central Europe. H has only been detected in Central Africa. J has been reported exclusively in Central America. K has recently been identified in the Democratic Republic of Congo and Cameroon.

This list is not exhaustive, for more subtypes are constantly being discovered, and migrating populations are shaping new patterns of infection. Of particular concern are HIV-1 clades C and A, as well as the A/G and A/E recombinant forms, which represent the predominant subtypes in Africa and Asia where HIV disease is dangerously out of control.

In sharp contrast, the other genre of retrovirus, HIV-2, has not spread much beyond West Africa, where it is presently endemic. Some sporadic cases have been observed elsewhere in Africa but the virus appears to be significantly less pathogenic than HIV-1.

HIV-1 clades are phylogenetically classified on the basis of the 20-50% differences in envelope (env) nucleotide sequences. The Env proteins of groups M and O may differ by as much as 30-50%. The N subtype, in turn, appears to be phylogenetically equidistant from M and O. Within M subgroups, inter-clade env variations differ by 2-30% whereas intra-clade variation of 10-15% is observed.

The pol region of HIV-1 is two to three times less divergent than env because this region encodes two critically important enzymes, RT and protease, which, if excessively mutated, render the virus inoperative. Gag sequences are even further intolerant of mutations, seeing as they encode for relatively inflexible core protein sequences.

Inter- and intra-clade variations within pol sequences are particularly relevant insofar as this region encodes RT and protease proteins, against which many antiviral drugs are directed. Variations in these regions may therefore affect drug susceptibility and development of drug resistance. Ethiopian clade C isolates differ (with respect to RT) from clade B by 6.8-10%, and intra-clade differences of 3.5-5.8% have been reported for strains from Africa, India and South America.

The fact that any given percentage variation in nucleotide sequence translates into lower amino acid sequence variation is notable because many genetic mutations are silent. For instance, the 10% nucleotide divergence between RT sequences in clades E and B yields only a 7% divergence in amino acid residues.

Not only do env genes vary substantially from clade to clade, but so do the long terminal repeat (LTR) sequences, which contain transcriptional promoters of HIV replication. Each clade has its own LTR copy number as well as an exact nucleotide sequence of enhancer and promoter structures, despite the uniformity in other LTR features, i.e. Sp1 sites, TATA box and TAT-responsive element. Moreover, diversity is seen in numbers of transcriptional promoters. These include the NF-κB binding sites (three to four in C, two in B and just one in E), as well as in sequences upstream of NF-κB sites, such as the nef-overlapping USF gene, which is incident only in clade B, and the AP-1 transcriptional factor binding site (which exists as one site in subtypes C, E and G, two in A and F, and none in B or D). The −170 region of U3, containing a specific motif for the NF-IL6 transcriptional factor (C/EBP-B), is harboured by clade B but not by A, C, D or O. This factor transactivates the HIV-1 LTR in cells of monocytic origin. Additionally, subtype discrepancies arise between the negative regulatory element (NRE) seen in clades C, D and E versus that detected in clade B.

Recent experiments indicate that the sequence of the viral regulatory protein, Nef, also differs between HIV-1 clades, ranging in variation from 14.4% to 23.8%, with the closest Nef configurations being those of B and D. The clinical implications of Nef sequence diversity are currently unknown but potentially great, given the recent observation that Nef sequences may change in clade B-infected patients as a function of disease progression.

Lastly, there is evidence that other regulatory and accessory HIV-1 genes may play an important role in subtype diversity. This relates partly to the fact that clade C contains a uniquely truncated Rev protein and an enlarged Vpu product, as well as the finding that clade D expresses a Tat protein with a C-terminus deletion.

In preferred embodiments, the present invention provides for nucleic acid and MVA vectors containing HIV-1 Clade C, as well as Circulating Recombinant Form 007, also known as HIV_{CHN.AD}, which also contains segments of clade B) that is the dominant subtype in Yunnan Province. Optionally, other clades may also be used in alternative embodiments.

Codon optimization has previously been described in WO 99/41397. Different cells differ in their usage of particular codons. This “codon bias” corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Codon optimization has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. Codon optimization also overcomes the Rev/RRE requirement for export, rendering optimised sequences Rev independent. Codon optimization also reduces homologous recombination between different constructs within the vector system (for example between the regions of overlap in the gag-pol and env open reading frames). The overall effect of codon optimization is therefore a notable increase in viral titre and improved safety.

The present invention involves the strategy of direct injection of DNA encoding viral antigens into skin or muscle. Local cells then take up the plasmids and express the foreign proteins themselves, essentially manufacturing the vaccine immunogens in situ. Optionally, utilizing an MVA prime boost enhances the immunogenic response in a subject at risk for an HIV infection or an HIV-related disease. This approach is economic and versatile. More important, however, is the potential for efficacy in vivo.

The invention also encompasses the use of the nucleic acid vectors to stimulate immune responses in subjects who are already infected with the virus. Further, the vectors of the invention can be used to generate antibodies either against the HIV sequences provided in the vectors, or against pre-existing circulating HIV sequences in the infected individual. Accordingly, the invention further envisions the use of antibodies generated using the disclosed vectors in diagnostic kits for HIV or HIV-related diseases.

Pharmaceutically acceptable carriers are determined in part by the particular composition being administered as well as by the particular method used to administer the compound. The present invention encompasses delivery of pharmaceutical compositions comprising nucleic acids, and optionally in combination with an MVA viral boost. In an alternative embodiment, MVA may be administered separately, without nucleic acid administration. Accordingly, there are a variety of suitable formulations of pharmaceutical compositions of these nucleic acids (see, for example, Remington's Pharmaceutical Sciences, 17^{th} ed. 1989). Administration can be by any convenient manner, for example, by injection, oral administration, inhalation, transdermal application, or rectal administration. When mucosal administration is used, it is possible to use oral, ocular or nasal routes.

Formulations suitable for parenteral administration, such as, for example, by intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of the invention, compositions can be administered, for example, by intravenous infusion, orally, topically, intraperitoneally, intravesically, or intrathecally. Parenteral administration is the preferred method of administration. The formulations can be presented in unit- or multi-dose sealed containers, such as ampules and vials.

A purified vaccine solution is prepared for administration by methods known in the art, which can include filtering to sterilize the solution, diluting the solution, adding an adjuvant, and stabilizing the solution. The vaccine can be lyophilized to produce a vaccine against HIV in a dried form for ease in transportation and storage. Further, the vaccine may be prepared in the form of the priming vaccines ADVAX I and ADVAX II alone or combined, and the booster vaccine ADMVA alone, or may contain at least one other antigen as long as the added antigen does not interfere with the effectiveness of the priming or booster vaccines, and the side effects and adverse reactions are not increased additively or synergistically. The recombinant poxvirus or immunogens may be in admixture with a suitable carrier, diluent, or excipient such as sterile water, physiological saline, or the like. The compositions can also be lyophilized or frozen. The compositions can contain auxiliary substances such as wetting or emulsifying agents, pH buffering agents, adjuvants, preservatives, and the like, depending upon the route of administration and the preparation desired.

Pharmaceutically acceptable adjuvants, such as complete or incomplete Freund's adjuvant, RIBI (muramyl dipeptides), ISCOM (immunostimulating complexes), cholera toxin B, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants that may protect the nucleic acid and/or viral vector from rapid dispersal by sequestering it in a local deposit, or they may contain substances that stimulate the host to secrete factors that are chemotactic for macrophages and other components of the immune system. The immunization schedule may or may not involve two or more administrations of the polypeptide, spread out over several weeks.

The invention comprehends a method for inducing an immunological or protective immune response against HIV in an animal comprising or consisting essentially of administering to the animal the immunogenic or vaccine composition.

The invention further comprehends a prime-boost immunization or vaccination against HIV, wherein the priming is done with (a) DNA vaccine(s) or immunological or immunogenic composition(s) that contains or consists essentially of (a) nucleic acid molecule(s) encoding and express(es) in vivo a HIV immunogen, antigen or epitope and the boost is done with (a) vaccine(s) or immunological or immunogenic composition(s) that is a HIV inactivated or attenuated or subunit (antigen, immunogen and/or epitope) preparation(s) and/or (a) recombinant or modified virus vaccine or immunological or immunogenic composition(s) that contains or consists essentially of (a) nucleic acid molecule encoding and express(es) in vivo (a) HIV immunogen(s), antigen(s) or epitope(s). Thus, the invention provides a prime-boost immunization or vaccination method against HIV, such as a prime-boost immunization or vaccination which comprises or consists essentially of or consists of administering to a target species animal (a) DNA vaccine(s) or immunological or immunogenic composition(s) of the invention (that contains or consists essentially of nucleic acid molecule(s) encoding and express(es) in vivo HIV antigen(s), immunogen(s) or epitope(s)) (as the prime) and thereafter administering (as the boost) administering a recombinant or modified virus vaccine or immunological or immunogenic composition that contains or consists essentially of nucleic acid molecule(s) encoding and express(s) in vivo HIV immunogen(s), antigen(s) or epitope(s), advantageously (a) recombinant vaccine or immunological or immunogenic composition(s) that expresses the HIV immunogen, antigen or epitope in vivo. The boost is advantageously matched to the prime, e.g., the boost contains or consists essentially of or expresses at least one antigen, epitope or immunogen that is expressed by the prime.

The methods of administration can comprise, consist essentially of or consist of the administration of an effective quantity of an immunogenic composition or vaccine according to the invention. One or more administrations can take place, such as two administrations. Compositions in forms for various administration routes are envisioned by the invention. The effective dosage and route of administration are determined by known factors, such as age, sex, weight, and other screening procedures which are known and do not require undue experimentation. Dosages of each active agent can be as in herein cited documents (or documents referenced or cited in herein cited documents) and/or can range from one or a few to a few hundred or thousand micrograms, e.g., 1 μg to 1 mg, for a subunit immunogenic, immunological or vaccine composition.

The amounts (doses) administered in the priming and the boost and the route of administration for the priming and boost can be as herein discussed, such that from this disclosure and the knowledge in the art, the prime-boost regimen can be practiced without undue experimentation. Furthermore, from the disclosure herein and the knowledge in the art, the skilled artisan can practice the methods, kits, etc. herein with respect to any of the herein-mentioned target species.

Vaccines or immunogenic compositions can be injected by a needleless, liquid jet injector or powder jet injector. For plasmids, it is also possible to use gold particles coated with plasmid and ejected in such a way as to penetrate the cells of the skin of the subject to be immunized (Tang et al., Nature 1992, 356, 152-154). Other documents cited and incorporated herein may be consulted for administration methods and apparatus of vaccines or immunogenic compositions of the invention. The needleless injector can also be for example Biojector 2000 (Bioject Inc., Portland Oreg., USA).

Advantageously, the immunogenic compositions and vaccines according to the invention comprise or consist essentially of or consist of an effective quantity to elicit an immunological response and/or a protective immunological response of one or more expression vectors and/or polypeptides as discussed herein; and, an effective quantity can be determined from this disclosure, including the documents incorporated herein, and the knowledge in the art, without undue experimentation.

In the case of immunogenic compositions or vaccines based on a plasmid vector, a dose can comprise, consist essentially of or consist of, in general terms, about in 10 μg to about 2000 μg, advantageously about 50 μg to about 1000 μg. The dose volumes can be between about 0.1 and about 2 ml, preferably between about 0.2 and about 1 ml.

Recombinant vectors can be administered in a suitable amount to obtain in vivo expression corresponding to the dosages described herein and/or in herein cited documents. For instance, suitable ranges for viral suspensions can be determined empirically. The viral vector or recombinant in the invention can be administered to a subject or infected or transfected into cells in an amount of about at least 10³ pfu; more preferably about 10⁴ pfu to about 10¹⁰ pfu, e.g., about 10⁵ pfu to about 10⁹ pfu, for instance about 10⁶ pfu to about 10⁸ pfu, per dose, for example, per 2 ml dose. If more than one gene product is expressed by more than one recombinant, each recombinant can be administered in these amounts; or, each recombinant can be administered such that there is, in combination, a sum of recombinants comprising these amounts. In recombinant vector compositions employed in the invention, dosages can be as described in documents cited herein or as described herein or as in documents referenced or cited in herein cited documents. For instance, suitable quantities of each DNA in recombinant vector compositions can be 1 μg to 2 mg, preferably 50 μg to 1 mg. Documents cited herein (or documents cited or referenced in herein cited documents) regarding DNA vectors may be consulted by the skilled artisan to ascertain other suitable dosages for recombinant DNA vector compositions of the invention, without undue experimentation.

However, the dosage of the composition(s), concentration of components therein and timing of administering the composition(s), which elicit a suitable immunological response, can be determined by methods such as by antibody titrations of sera, e.g., by ELISA and/or seroneutralization assay analysis and/or by vaccination challenge evaluation in a subject. Such determinations do not require undue experimentation from the knowledge of the skilled artisan, this disclosure and the documents cited herein. And, the time for sequential administrations can be likewise ascertained with methods ascertainable from this disclosure, and the knowledge in the art, without undue experimentation.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Construction of DNA Vaccines ADVAX I and II

The prophylactic vaccine regimen of the invention comprises two novel DNA vectors, followed by boosting with a Modified Vaccinia Ankara (MVA) recombinant expressing corresponding HIV-1 proteins. The genes used in the instant vaccines are derived from an HIV-1 clade C strain, Circulating Recombinant Form 007, or HIV_{CHN.AD}, which also contains small segments of Clade B that is the dominant subtype in Yunnan. The nef and tat gene products are expressed early in the viral life cycle, and may represent key targets for immunologic control of HIV-1 infection. In addition, the Gag, pol, and env structural genes were also selected. Therefore, both structural and regulatory genes were included in the DNA vaccine strategy of the present invention, designed for maximal inclusion of immunogenic epitopes.

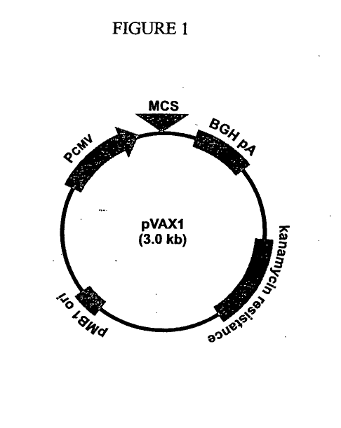

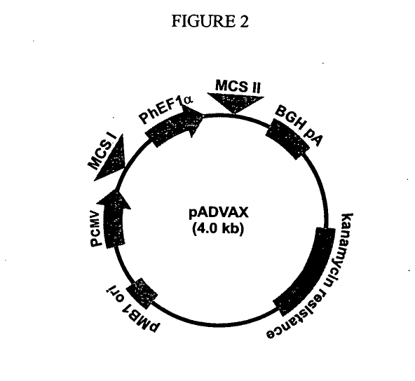

The DNA vaccines of the present invention are based on pVAX 1, a commercially available plasmid from Invitrogen® (FIG. 1). This vector was designed specifically for use in the development of DNA vaccines, and was constructed to be consistent with United States Food and Drug Administration (FDA) guidelines (Center for Biologics Evaluation and Research, FDA, 22 Dec. 1996, Docket No. 96N-0400). The original vector was modified, however, by inserting an additional promoter. PCR was used to amplify the human elongation factor 1α (hEF1α) promoter from pBudCE4.1, a commercially available vector (Invitrogen®). The promoter was cloned into the EcoRI/NotI sites of pVAX1 and the new construct was verified by sequencing. The hEF1αpromoter has been well-characterized by others (Najjar, S. M. et al. (1999) Gene 230: 41-5; Nishimura, Y. et al. (1999) Vaccine 18: 675-80; Wallich, R. et al. (2001) Infect. Immun. 69:2130-6). This alteration of pVAX 1, yielding pADVAX (FIG. 2), was found to permit independent, high-level expression of a second genetic insert. The bicistronic capacity of pADVAX is more potent (by 10- to 20-fold) than that achieved with use of an internal ribosomal entry site, or IRES (Martinez-Salas, E. (1999) Curr. Opin. Biotechnol. 10:458-64). Western blots showed that the level of protein expression from each gene under the dual promoters of pADVAX is comparable to that driven by the CMV promoter alone in pVAX1.

After constructing the pADVAX vector, the HIV viral genes were prepared for insertion. HIV-1 env and gag genes were synthesized to comprise codons optimal for mammalian expression. Codon optimization represents a facilitation of Rev/RRE-independent nuclear export (Schneider, R. et al. (1997) J. Virol. 71:4892-903; Kotsopoulou, E. et al. (2000) J. Virol. 74: 4839-52), and was consistently found to enhance expression of viral genes. Overlapping PCR was used to unite oligonucleotides (80- to 90-mers overlapping by 16-18) with sequences reflecting this ideal codon selection. In doing so, gene expression was enhanced by 100- to 1000-fold, as measured by ELISA or Western blot.

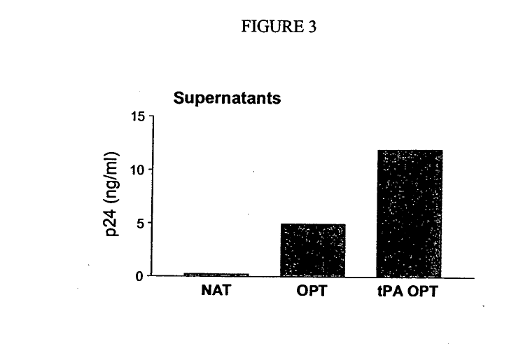

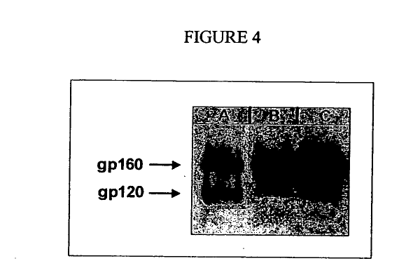

The genes were further modified by incorporating a tissue plasminogen activator (tPA) leader sequence (MDAMKRGLCCVLLLCGAVFVSAR) (SEQ ID NO: 1), replacing the native sequence of env and supplementing the gag gene. This sequence is thought to enhance expression in part by facilitating transport of protein from the endoplasmic reticulum (ER) to the Golgi apparatus (Haddad, D. et al. (1997) FEMS Immunol. Med. Microbiol. 18:193-202; Li, Z. et al. (1999) Infect. Immun. 67: 4780-6; Weiss, R. et al. (1999) Vaccine 18: 81524; Qiu, J. T. et al. (2000) J. Virol 74: 5997-6005). With this modification, gene expression was further enhanced by 3- to 5-fold. FIG. 3 shows the expression of gag of native (NAT), codon-optimized (OPT), and codonoplimized/tPA (tPA OPT) as measured by ELISA (Abbott Laboratories) that quantifies HIV-1 Gag (p24). Similar results were obtained with enhanced env expression provided by codon optimization and addition of the tPA leader sequence, as measured in a Western blot using a polyclonal antibody to the env gene product (FIG. 4). The results established that Env function is preserved with the genetic modifications. In a fusion assay involving HeLa cells bearing CD4/CCR5 (HIV-1 receptor/co-receptor), 293T cells transfected with a tPA-optimized env vector were capable of fusing to form syncytia (results not shown).

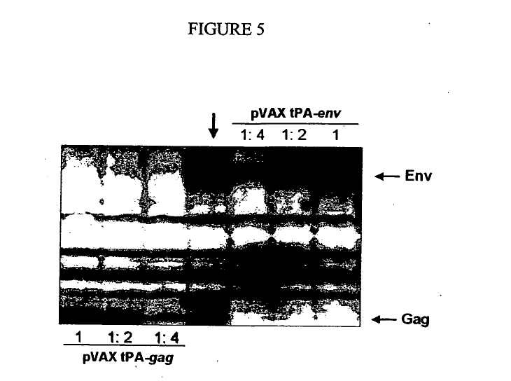

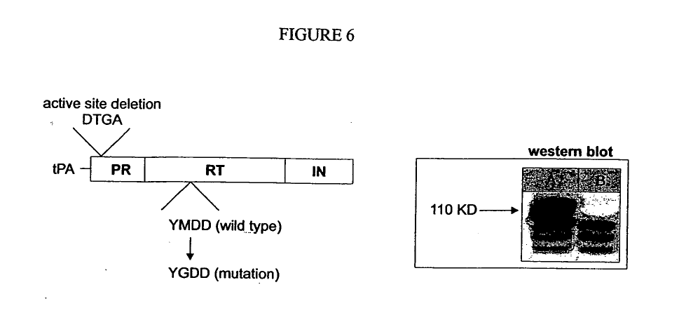

With the desired genetic modifications in place, two-HIV-1 genes were cloned into pADVAX to create the first vaccine, ADVAX I. Bicistronic expression was confirmed by Western blot (FIG. 5). The second vaccine, ADVAX II, was again constructed as described above, with overlapping PCR to unite codon-optimized oligonucleotides for synthesis of pol, nef; and tat However, additional measures were taken to ensure safety for in vivo use. First, a deletion in the active site of protease (PR) was included in the pol gene to prevent polypeptide processing (Loeb, D. D. et al. (1989) Nature 340: 307-400), a consequence that was confirmed by Western blot (FIG. 6). An additional cautionary step was taken to incorporate a point mutation in the active site of reverse transcriptase (RT), also in the pol gene (Wakefield, J. K. et al. (1992) J. Virol 66: 6806-12, Chao, S. F. et al. (1995) Nucleic Acids Res. 23: 803-10).

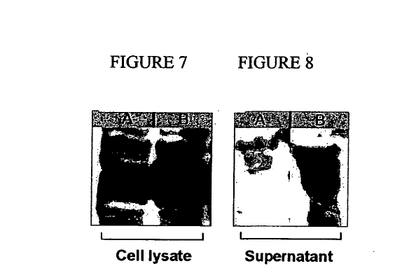

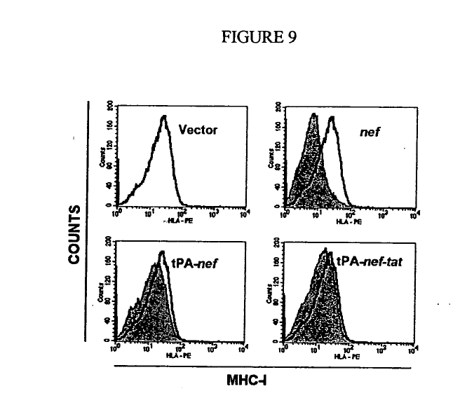

To incorporate all three genes into a single pADVAX-based vector, overlapping PCR created a nef-tat fusion gene. Both genetic sequences were kept intact, thereby preserving all immunogenic epitopes in the resultant fusion protein. As described before, a tPA leader sequence was added to both pol and nef-tat. The increased efficiency of expression and secretion achieved was verified by Western blot performed with both lysates and supernatants of 293T cells transfected with the relevant vectors (FIGS. 7-8). The antibody used was a polyclonal rabbit anti-Nef antibody (provided by Dr. Cecilia Cheng-Mayer). As with Pol, the safety of the nef-tat fusion protein for in vivo use was considered and addressed by the following analyses.

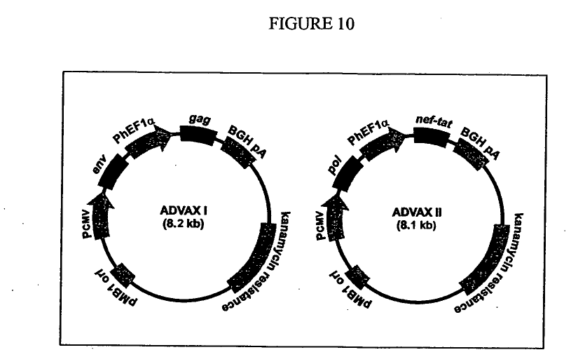

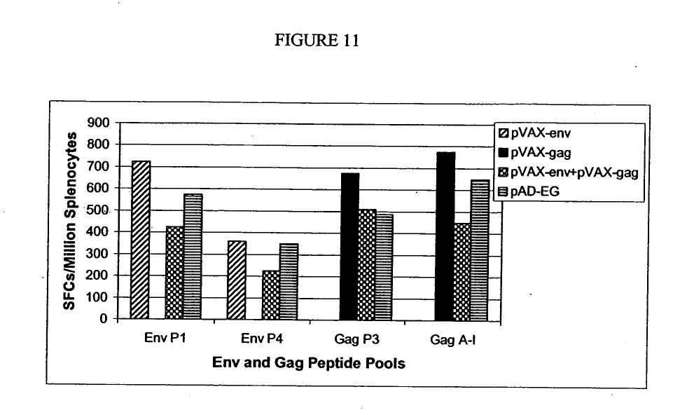

Nef is known to down-regulate both CD4 and MHC-class I surface expression (Collins, K. L. et al. (1998) Nature 391:397-401; Aiken, C. et al. (1994) Cell 76:853-64; Collins, K. L. et al. (1999) Immunol. Rev. 168:65-74), and tat has immunosuppressive effects, presumably by acting as a general transactivator (Goldstein, G. (1996) Nat. Med. 2:960-4; Garber, M. E. et al (1999) Curr. Opin. Immunol. 11: 460-5). Using flow cytometric analysis, however, we demonstrated that the Nef effect on MHC Class I expression is nullified by the tPA leader sequence (FIG. 9). Similarly, in the context of the nef-tat fusion protein, tat loses its ability to transactivate. This phenomenon is seen manifestly in the “MAGI” assay, which involves the use of HeLa cells that are engineered to express the 13-galactosidase gene in the presence of functional HIV-1 Tat (Kimpton, J. et al. (1992) J. Virol. 66:2232-9). With the addition of the X-gal (5-bromo-4-chloro-3-indolyl-β-D-galactopyranoside) substrate, the cells turn blue only if tat is active (results not shown). It can be deduced, therefore, that the Nef-Tat fusion protein generated by this vaccine will not have immunosuppressive effects in vivo. Indeed, even with the risk for general transactivation overlooked, it has been seen that DNA encoding wild-type HIV-1 tat is safe for use as a vaccine in immuno-compromised individuals (Calarota, S. A. et al. (1999) J. Immunol. 163:2330-8). The vectors are represented by FIG. 10.

### Example 2

### In Vivo Immunogenicity Assessment of ADVAX I and II-Cell Mediated Response

The determination of cell-mediated immune response to ADVAX I and II was evaluated with the ELISpot assay as described above (see also Hanke, T. et al. (1998) J. Gen. Virol 79: 83-90; Carvalho, L. H. et al. (2001) J. Immunol. Methods 252: 207-18; Tobery, T. W. et al. (2001) J. Immunol. Methods 254: 59-66; Novitsky, V. et al. (2001) J. Virol. 75: 9210-28). Beginning with a GLP-grade stock (Aldeveon, Fargo, N.D.) of ADVAX 1, 6-8 week-old female BALB/c mice were immunized. The vaccine was administered as 200 μg intramuscularly at weeks 0, 3 and 6. A total of 5 groups of 6 mice each were inoculated with the following constructs: pVAX1-env, pVAX1gag, pVAX1-env+pVAX1-gag, pVAX1 (control) and ADVAX I. Peptides represented specific epitopes as follows: Env 34 (VPVWKEAKTTLFCASDAKAY) (SEQ ID NO:3) is known to elicit a CD4+ cell-mediated response, Env 43 (RNVSSDGTYNETYNEIKNCS) (SEQ ID NO:4) elicits a CD8+ cell-mediated response, Gag 26 (TSNPPIPVGDIYKRWIILGL) (SEQ ID NO:5) elicits a CD4+ cell-mediated response and Gag A-1 (AMQMLKDTI) (SEQ ID NO:6 OR 2) elicits a CD8+ cell-mediated response.

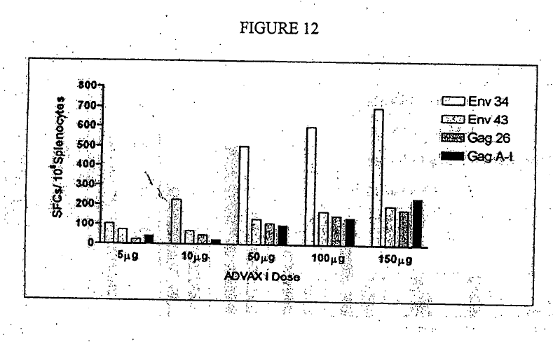

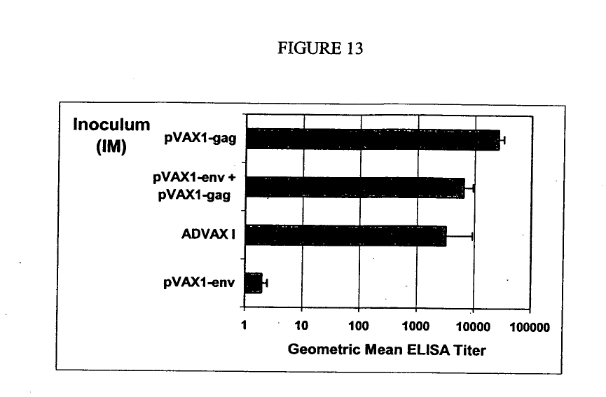

Two weeks after the third injection, the mice were sacrificed. Splenocytes were then pooled from each group and assayed using ELISpot for their ability to secrete interferon-γ (IFNγ) during re-stimulation in vitro with Env and Gag antigen-specific peptide pools (NIH AIDS Research and Reference Reagent Program). At the time of filing, only Gag peptides from a heterologous strain (HIV_{96ZM65.8}, Catalog No. 3993) were available. Similarly, we did not yet have a complete set of homologous Env peptides (HIV_{CHNAD}, Catalog No. 4974, 80% complete) at the time of our ELISpot assay. Nonetheless, the results revealed strong immune responses to both single promoter driven vectors (pVAX1-env and pVAX1-gag), each yielding approximately 700 spot-forming cells (SFC) per million splenocytes. The immune response induced by ADVAX I was comparably significant, with approximately 600 SFC/million splenocytes specific to both Env and Gag peptide pools detected. Predictably, the response to the pVAX1 control was nil and upon depletion of CD8+ cells from the splenocyte pools, no ELISpot response to Gag A-I was detected (FIG. 11). Overall, the cell-mediated immune responses were directed against at least 9 different epitopes, including ones specific for either CD8⁺ or CD4⁺Tcells (data not shown). No evidence of immunogenic synergy or interference between the two gene products of ADVAX I were detected. Dose escalation experiments revealed a clear dose-response effect (FIG. 12). For at least one epitope (Env 34), the quantitative ELISpot response seen for 150 μg was approximately seven times that for 5 μg. Nonetheless, the dose-response trend holds true for all epitopes tested, whether specific for CD4⁺ or CD8⁺ cell-mediated responses.

### Example 3

### Pre-Clinical In Vivo Immunogenicity Assessment

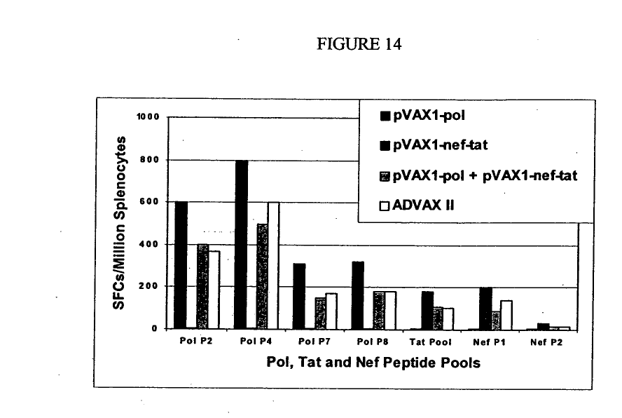

The following data supports the humoral immunogenicity of ADVAX I in vivo. Serum samples collected two weeks after the final (third) immunization to a mouse trial were tested for anti-Gag antibodies using ELISA. Although the highest titer was seen in the mice inoculated with pVAX1-gag, there was also a substantial titer in the group immunized with ADVAX I, which was comparable to the response displayed by the animals who received pVAX1-env+pVAX1-gag (FIG. 13). The serum samples collected from the ADVAX I group also demonstrated an antibody response to Env by Western blot. Similar in vivo studies were carried out with ADVAX II. Specifically, GLP-grade stock (Aldevron, Fargo, N.D.) of ADVAX II was used to immunize 6-8 week-old female BALB/c mice. The vaccine was administered as a 200 μg IM injection at weeks 0, 3 and 6. A total of 5 groups of 5 mice each were inoculated with the following constructs: pVAX1-pol, pVAX1-nef-tat, pVAX1-pol+pVAX1-nef-tat, pVAX1 (control) and ADVAXII (the only dual-promoter vector).

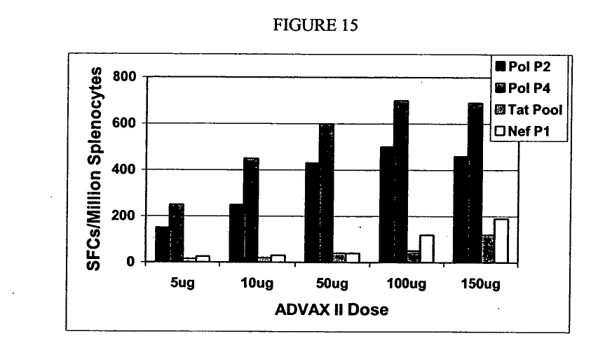

Two weeks after the third injection, the mice were sacrificed. Splenocytes were then pooled from each group and assayed using ELISpot for their ability to secrete interferon-γ during re-stimulation in vitro with pol, tat and nef derived peptide pools. Of note, no Clade C peptides were available at the time of the assay. The antigen-specific peptides consisted of 15-mers based on Clade B consensus sequences (NIH AIDS Research and Reference Reagent Program: Tat Catalog No. 5138, Nef Catalog No. 5189 and Pol Catalog No. 6208). Nonetheless, as in the ADVAX I trial, we saw comparably good responses with both the single-gene vectors and the dual-promoter vaccine. Responses to Pol pools, for example, were best for pVAX1 pol alone (300-800 SFC per million splenocytes, depending on pool). For pVAX1 pol+pVAX1 nef-tat; results ranged from 180 to 500 SFC per million splenocytes, and for ADVAX II, the response was between 180 and 600 SFC per million splenocytes. With a Tat pool, the response was −180 SFC for pVAXI-nef tat, and −100 SFC for both pVAX1 pol+ pVAX1 nef-tat and ADVAX II. Using Nef pools, the response was 30-200 SFC for pVAX1 nef-tat and 20-150 SFC for both pVAX1 pol+ pVAX1 nef-tat and ADVAX II (FIG. 14).

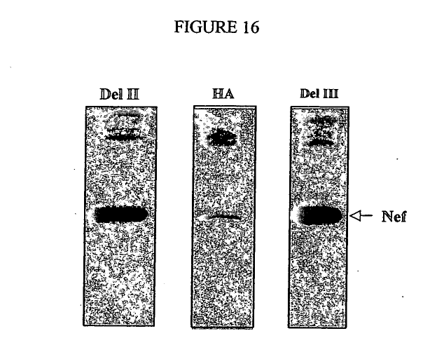

We additionally performed a dose-escalation study using ADVAX II, demonstrating a clear dose response effect (FIG. 15). Mice were injected IM with 5 μg, 10 μg, 50 μg, 100 μg or 150 μg of DNA at weeks 0, 3 and 6. After sacrifice at week 8, splenocytes were pooled and re-stimulated in vitro with peptides derived from the Clade B consensus sequence. Pol responses ascended in accordance with dose, from 250-050 SFC at 10 μg to 500-700 SFC at 100 μg. The response at 150 μg was comparable to that of 100 μg. Nef responses ranged from −20 SFC to −200 SFC, and Tat responses from −25 SFC to −100 SFC.

An in vivo trial of ADVAX I+II administered together as a combination-inoculum was conducted. Groups of mice received IM injections of 5 μg, 10 μg, 50 μg, 100 μg or 150 μg of ADVAX I+II at weeks 0, 3 and 6. The control group received a mixture of 50 μg each of pVAX1 gag, pVAX1 env, pVAX1 pol and pVAX1-nef-tat Two weeks following the final immunization, the mice were sacrificed, and splenocytes pooled from each group were assayed using ELISpot for interferon-γ release. Env-, Gag- and Pol-specific peptides containing CD4+ and CD8+ T cell epitopes were used for in vitro re-stimulation, as were autologous subtype C Tat and Nef sequences. As seen in separate ADVAX I and ADVAX II trials, the results of this combination-inoculum trial show comparably good responses to both vaccine vectors. The mice inoculated with ADVAX I+II have antigen-specific responses to all peptides (peptide pools) tested, and the response is induced in a dose-dependent manner (Table 1).

TABLE 1
Antigen-specific interferon-γ ELISpot Responses to a Combination
Inoculum and Control

| | IFN-γ spot-forming cells (SFC)/10⁶ splenocytes | | | | | | | | | |
|---|---|---|---|---|---|---|---|---|---|---|
| Vaccine | Gag | Gag | Env | Env | Pol | Pol | Pol | Tat | Nef | Nef |
| Dose | 26 | A-I | 34 | T-I | 223 | YLI | VGI | pool | Pool1 | Pool2 |
| pVAX1-gag + | 120 | 150 | 350 | 700 | 70 | 500 | 500 | 110 | 400 | 300 |
| pVAX1-env + | | | | | | | | | | |
| pVAX1-pol + | | | | | | | | | | |
| pVAX1-nef-tat | | | | | | | | | | |
| (50 μg each) | | | | | | | | | | |
| ADVAX | 112 | 252 | 400 | 700 | 117 | 700 | 830 | 120 | 225 | 370 |
| I + II | | | | | | | | | | |
| (150 μg | | | | | | | | | | |
| each) | | | | | | | | | | |
| ADVAX | 70 | 210 | 400 | 600 | 100 | 550 | 500 | 70 | 200 | 330 |
| I + II | | | | | | | | | | |
| (100 μg | | | | | | | | | | |
| each) | | | | | | | | | | |
| ADVAX | 30 | 100 | 220 | 500 | 84 | 400 | 420 | 50 | 140 | 160 |
| I + II (50 μg | | | | | | | | | | |
| each) | | | | | | | | | | |
| ADVAX | 30 | 60 | 180 | 250 | 70 | 275 | 375 | 30 | 110 | 140 |
| I + II (10 μg | | | | | | | | | | |
| each) | | | | | | | | | | |
| ADVAX | 10 | 30 | 110 | 180 | 60 | 250 | 370 | 20 | 70 | 140 |
| I + II (5 μg | | | | | | | | | | |
| each) | | | | | | | | | | |

Of note:
Number of spots was normalized per million splenocytes and averaged for each sample and antigen based on duplicate wells for both of the two cell input levels. Gag 26, Env 34, and Pol 223, contain CD4 epitopes, whereas Gag A-I, Env T-I, Pol YLI, and Pol VGI contain CD8 epitopes. Peptide pools used for Tat and Nef are derived from autologou subtype-C sequences.

Of note, a particularly robust response was seen in this trial on in vitro re-stimulation of splenocytes with subtype C-derived Nef and Tat peptide pools, in contrast to the milder response seen earlier in the ADVAX I trial. This discrepancy is likely attributable to the differences between heterologous and autologous proteins. Thus, we believe that the nef-tat fusion gene in the context of ADVAX II can, in fact, induce very potent immune responses. Furthermore, the combination-inoculum trial demonstrates no detectable interference among the different antigen-specific responses measured by interferon-γ ELISpot.

### Example 4

### Construction of a Recombinant MVA Viral Vector as an HIV-1 Vaccine

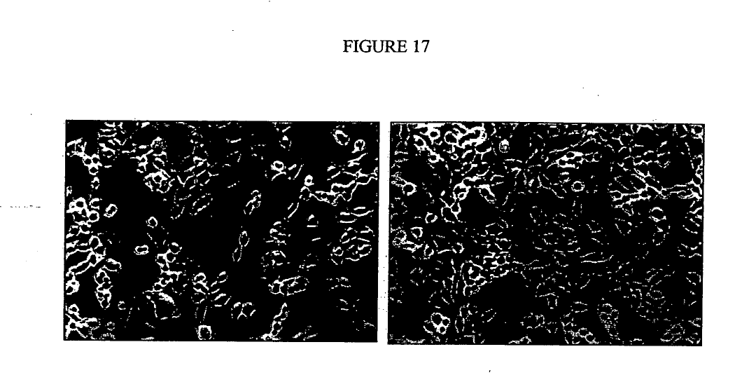

The MVA shuttle vectors we used were originally obtained from Dr. Bernard Moss of the National Institutes of Health. The vectors were further modified to be consistent with United States Food and Drug Administration (FDA) guidelines. We first modified the original vector by removing reporter or drug-resistant genes that we did not want to introduce into humans. We then evaluated three MVA insertion sites (del II, del III and HA) for the expression of HIV-1 proteins. We found that del II and del III are superior to the HA site (FIG. 16) respect to gene expression as discerned by Western blot. Moreover, we noted that vaccinia-specific promoters drive foreign gene expression in the following order: pSYN>pH5>p7.5. Thus, we chose del II and del III sites as well as vaccinia promoter pSYN and pH5 for our vaccine construction. This strategy was adopted to ensure high-level expression of HIV-1 proteins.

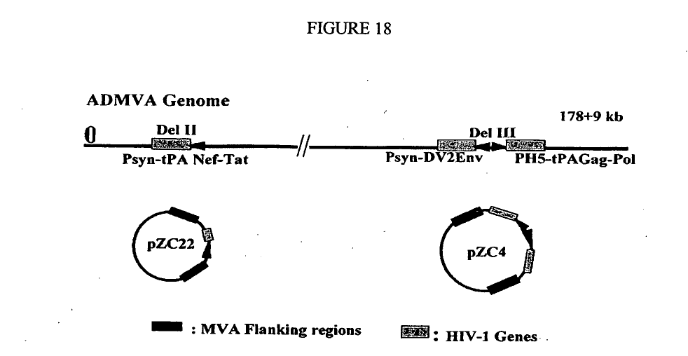

The expression of HIV-1 genes was evaluated in the MVA system. Although sequence “humanization” helps the enhancement of protein expression in DNA vaccines, it did not offer any advantage to the expression of HIV-1 proteins by MVA as observed in comparison of humanized and wild-type nef genes inserted into the del II site by Western blot. Therefore, wild-type HIV-1 sequences were chosen for constructing our vaccines. Furthermore, MVA cannot tolerate full-length HIV envelope protein, probably due to its toxicity to the vaccinia virus (Amara, R. R., et al. (2001) Science 292: 69-74; Barouch, D. H., et al. (2001) J Virol 75: 5151-8; Men, R., et al (2000) Vaccine 18: 3113-22; Ourmanov, I., et al (2000) J Virol 74: 2740-51; Takahashi, H., et al (1988) Proc Natl Acad Sci USA 85: 3105-9). Thus, a truncation is usually introduced into the carboxyl terminus of the HIV-1 gp41 region to generate a live recombinant HIV-1-MVA. Taking these findings into consideration, the viral envelope sequence was modified by introducing deletions in its variable (V) regions. In comparison to wild type, deletion in gp120 V2 regions (DV2) allowed the recombinant MVA to reach a higher titer, i.e. >10⁸TCID₅₀/ml. This modification preserved all immunogenic epitopes in gp41. The DV2 envelope may serve as a better immunogen to induce broad neutralization antibodies against HIV-1 because of enhanced exposure of certain key antibody epitopes (Barnett, S. W., et al. (2001) J Virol 75: 5526-40; Cherpelis, S., et al (2001) Immunol Lett 79: 47-55; Cherpelis, S., et al (2001) J Virol 75: 1547-50; Donnelly, J. J., et al. (2002) Science 297: 1277-8; discussion 1277-8; Ly, A., and L. Stamatatos. (2000) J Virol 74: 6769-76; Stamatatos, L., et al. (2000) AIDS Res Hum Retroviruses 16: 981-94). Of note, DV2 also eliminated a vaccinia transcription termination signal that could affect the expression of full-length envelope (Moss, B., et al. (1996) Adv Exp Med Biol. 397: 7-13; Moss, B. (1996) Proc Natl Acad Sci USA 93:11341-8). Importantly, the functional properties of DV2 envelope were preserved, despite the modifications. In a fusion assay involving HOS cells that bear CD4/CCR5 (HIV-1 receptor/co-receptor), 293T cells transfected with a modified DV2 env vector were capable of fusing to form syncytia (FIG. 17).

Since desirable insertion sites are limited for MVA, overlapping PCR created gag-pol and nef-tat fusion genes. In doing so, both genetic sequences were retained intact, thereby preserving all immunogenic epitopes in the resultant fusion proteins. Consistent with the approach to enhance expression in our plasmid DNA vaccine, the genes were further modified by incorporating a tissue plasminogen activator (tPA) leader sequence (amino acids: MDAMKRGLCCVLLLCGAVFVSAR), supplementing the gag-pol and nef-tat fused genes.

As described for the DNA vaccine, additional efforts were taken to ensure safety for in vivo use. A deletion in the active site of protease (PR) was introduced in the pol gene, such that polypeptide processing was prevented (Loeb D D, et al. (1989) Nature August 3; 340(6232): 397-400). The deletion in protease (DTGA) comprises amino acids 25-28 of the wild-type gene. A point mutation was also incorporated in the active site of reverse transcriptase (RT), so that retroviral reverse transcription was inactivated (Wakefield, J. K., et al. (1992) J. Virol. 66(11): 6806-12; Chao S. F., et al (1995) Nucleic Acids Res 23(5): 803-10). The point mutation in reverse transcriptase (YMDD to YGDD) corresponds to position 184 of the wild-type gene. The nef and tat genes were introduced as a nef-tat fusion gene including a tPA leader sequence. When evaluated in the MAGI assay, this tPA nef-tat fusion gene, in the context of the plasmid DNA vaccine candidate, was shown to have lost the transactivating function normally associated with the expression of native tat. The effect of nef on down-regulation of MHC class I surface expression was nullified by the introduction of the tPA leader sequence as demonstrated by flow cytometric analysis of 293T cells, comparing cells transfected with a plasmid expressing tPA nef-tat, versus a construct expressing native nef alone.

To incorporate all five genes into a single recombinant MVA virus, a dual-promoter shuttle vector, pZC1, was constructed. Using this novel vector, both env and gag-pol genes were inserted into MVA deletion III by homologous recombination.

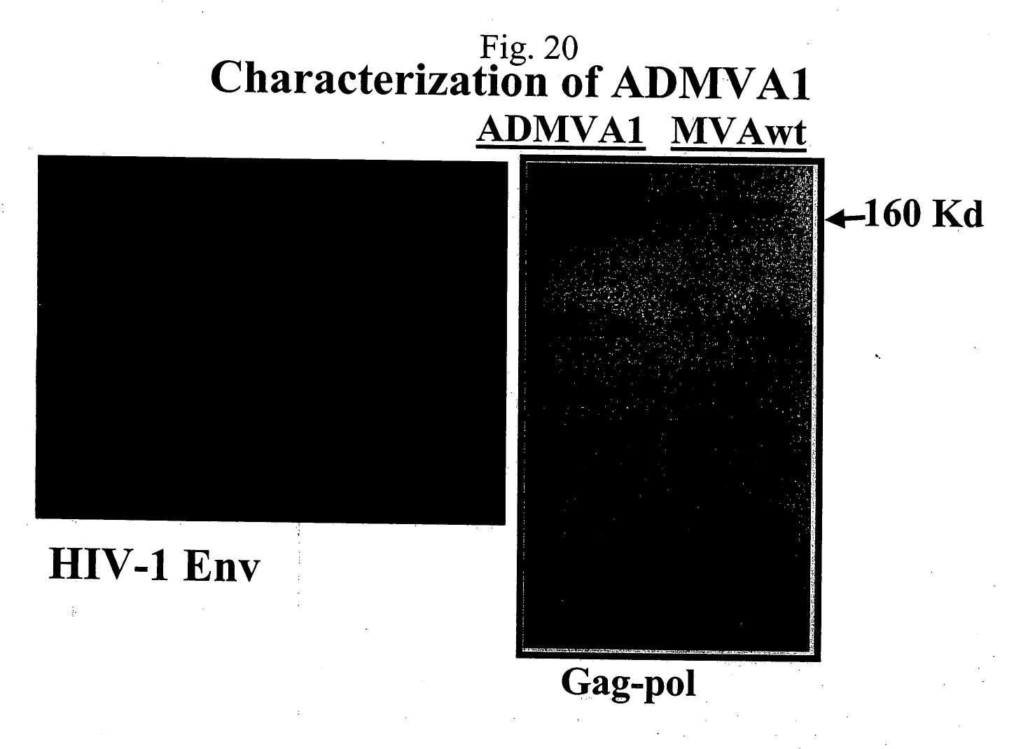

The MVA shuttle plasmid pLW7, generously provided by Drs. Moss and Wyatt at NAIAD at the National Institutes of Health, contains the SYN promoter and directs recombination into MVA deletion III. From the data obtained to rank the efficiency of vaccinia-specific promoters, the pLW7 plasmid was modified to create a novel dual promoter insertion plasmid by the addition of a cloning site under the control of the H5 promoter. Since pZC1 harbors two distinct promoters, the potential problem of promoter competition was not deemed to be an issue. The dual promoter insertion plasmid ZC1 was used to construct the shuttle plasmid pZC4 containing DV2Env under control of the SYN promoter and tPA gag-pol under the control of the H5 promoter. Instead of delivering one foreign gene, this new insertion vector pZC1 was constructed to deliver two foreign genes into the Del III region of the MVA genome. Therefore, pZC1 can deliver env and gag-pol, each under the control of separate and different vaccinia promoter, but in the same insertional cassette, into the Del III region of MVA (FIG. 19).

Because envelope immunostaining has proven to be sensitive and reliable, env was then used as a surrogate marker to screen for the presence of gag-pol, which is otherwise difficult to detect by itself. Thus, cells positive for envelope staining had gag-pol gene integrated on the genome as well. After multiple rounds of enrichment, gag-pol expression can then be confirmed by Western blot, which is more sensitive than in situ immunostaining.

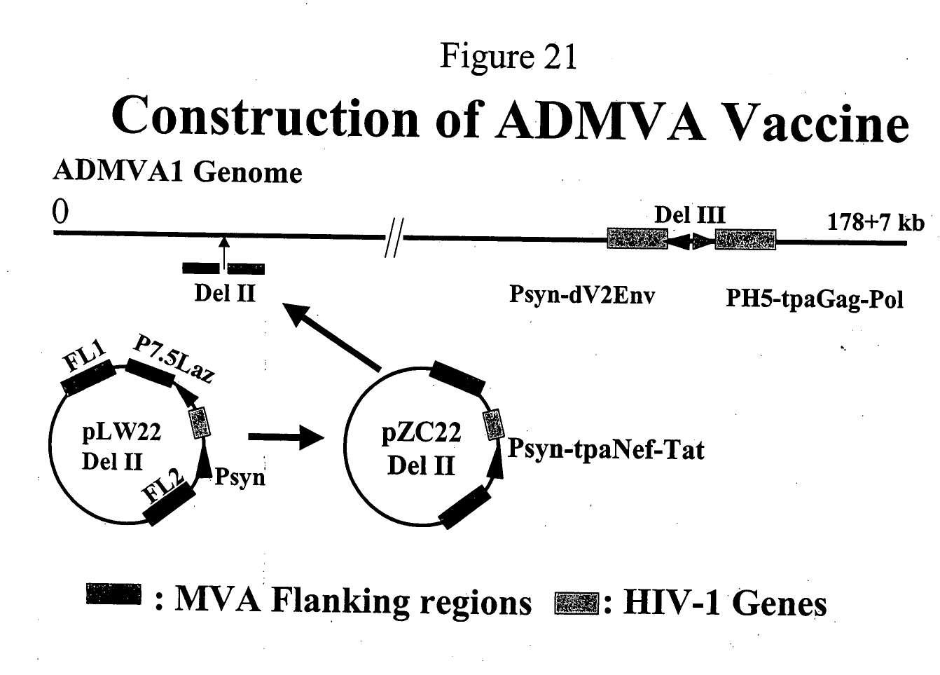

As described earlier, both HIV-1 env and gag-pol were inserted under separate promoters as pZC4. The env-gag-pol pZC4 was inserted into the Del III region of wild-type MVA by homologous recombination. The recombinant env-gag-pol MVA was identified by immunostaining using an anti-Env antibody, and further confirmed by testing gag-pol expression using Western Blot analysis. Thus, both genes are expressed in the dual-promoter construct (FIG. 20). The recombinant env-gag-pol MVA strain (“ADMVA”) was further propagated through enrichment/selection with an anti-Env antibody.

CEF cells infected with parental strain MVA P585 were transfected with plasmid pZC4 (env/gag-pol) to generate the recombinant MVA expressing DV2env and tPA gag-pol by homologous recombination. Harvested cell lysate from the transfected culture was sonicated, diluted, and plated on CEF cells. Resultant monolayers were immunostained and individual foci were picked. Positive foci were identified by staining with inactivated human anti-Env sera (Km94). The foci were transferred into a tube containing DMEM with 2% FCS. Several cycles of freezing thawing were performed to disrupt cells and release bound virus. The contents were clarified by centrifugation. The supernatant was aspirated and virus expanded by infecting 150 ml TC flasks containing CEF cells. Infected cells were harvested after 48 hours and bound virus released by disrupting the cells. The virus was purified on a 36% sucrose cushion by ultracentrifugation and titer was then determined. Based on the results of the titration, the virus was diluted by limiting dilution and the next round of foci purification undertaken. The procedure for foci purification was performed successively 11 times. The selected isolate was named ZC4PCRE11/12. Expression of the inserted DV2env gene was confirmed by immunostaining while gag-pol expression was confirmed by Western blot analysis.

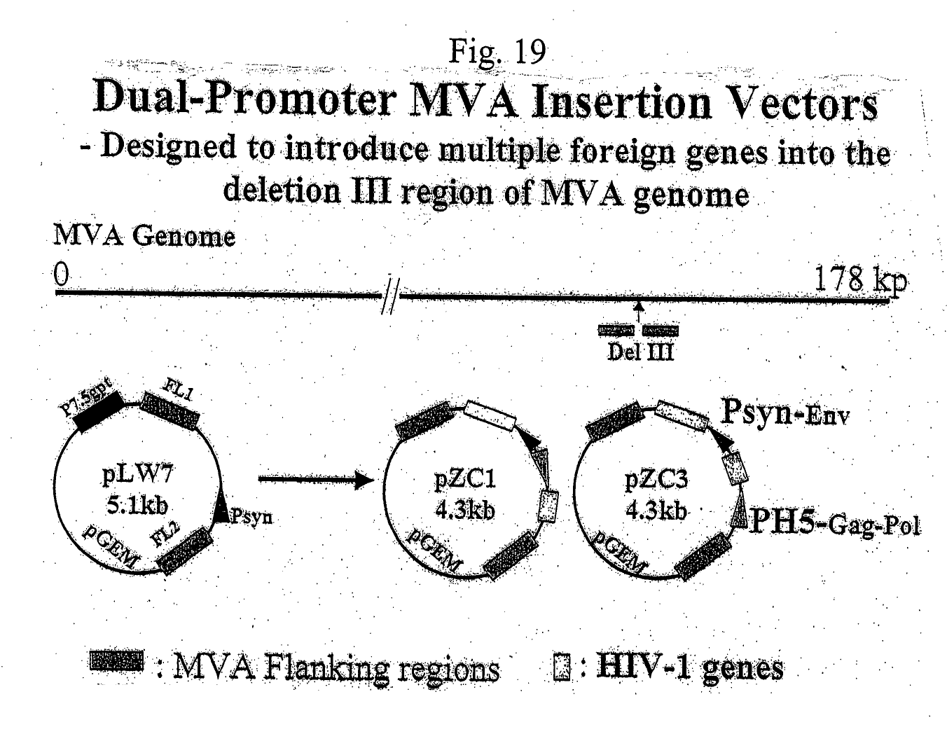

The multigenic recombinant, termed ADMVA, was generated by homologous recombination between CEF cells infected with the above recombinant MVA clone and the shuttle plasmid pZC22 that directed insertion of the tPA modified nef-tat fusion gene into MVA deletion II, more than 120 kbp upstream of the del III region (FIG. 18). Harvested cell lysates from the transfected culture were sonicated, diluted, and plated on CEF cells. The resultant monolayers were immunostained and individual foci were picked. Positive foci were identified using a double-staining selection technique using rabbit anti-nef and inactivated human anti-env sera. Nine successive rounds of foci purification were performed, as described above. Prior to characterization, the final isolate underwent further random expansion through 5 passages resulting in the ADMVA Research Seed stock with a titer equal to 2.15×10⁶ TCID₅₀/ml.

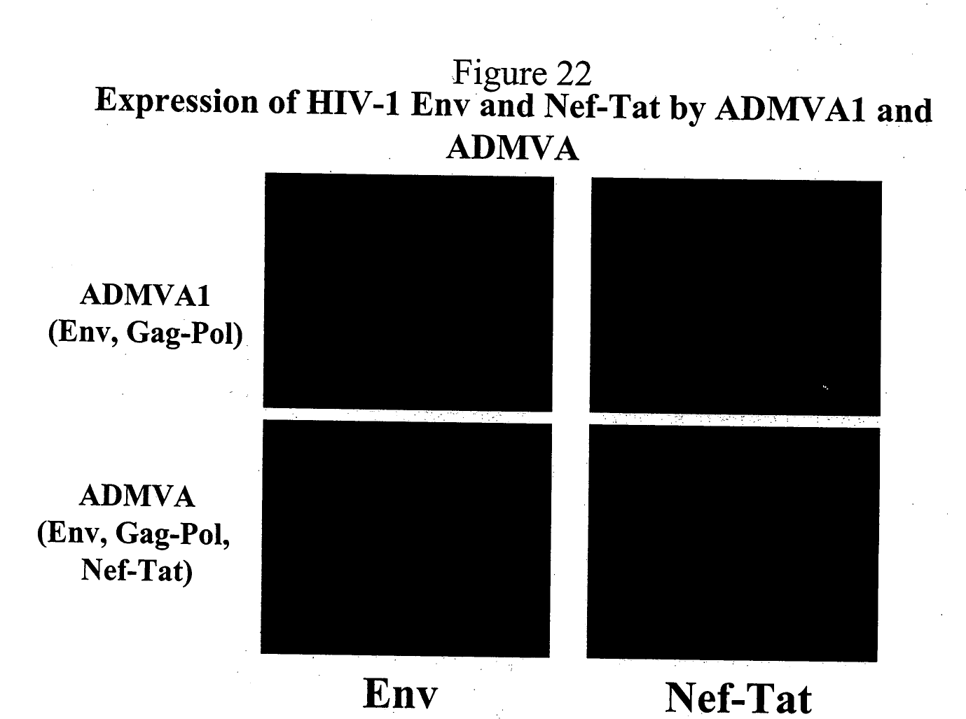

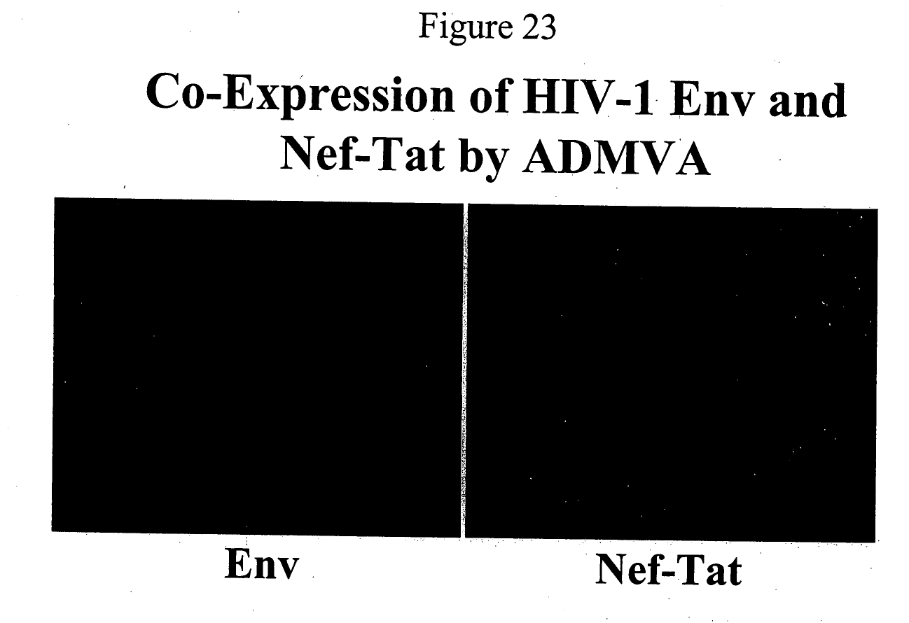

The MVA shuttle plasmid pLW22 contains the SYN promoter and directs recombination into MVA deletion II (FIG. 21). The del II region is more than 120 kbp upstream of the del III region. The pLW22 plasmid was modified to yield the shuttle plasmid pZC22 containing tPA nef-tat fusion gene under control of the SYN promoter. The identities of shuttle plasmids pZC4 and pZC22 were confirmed by restriction enzyme analysis. PCR analysis was undertaken to confirm the identity of the inserted transgenes. The modification eliminated the presence of the reporter gene. In theory, multiple HIV-1 genes can be recombined into a single MVA genome by using both pZCI and pZC22 vectors. To use five HIV-1 genes for the DNA vaccination, a second variant of ADMVA was constructed. HIV-1 nef-tat genes were inserted into pZC22, and this nef-tat pZC22 was introduced into the del II region of plaque-purified ADMVA by homologous recombination. The recombinant ADMVA was identified by double-immunostaining using anti-Env and anti-Nef antibodies (FIGS. 22-23). The recombinant ADMVA strain was plaque-purified through selection with anti-Env and anti-Nef antibodies.

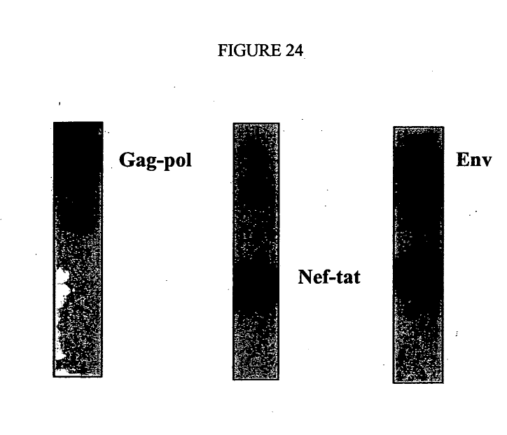

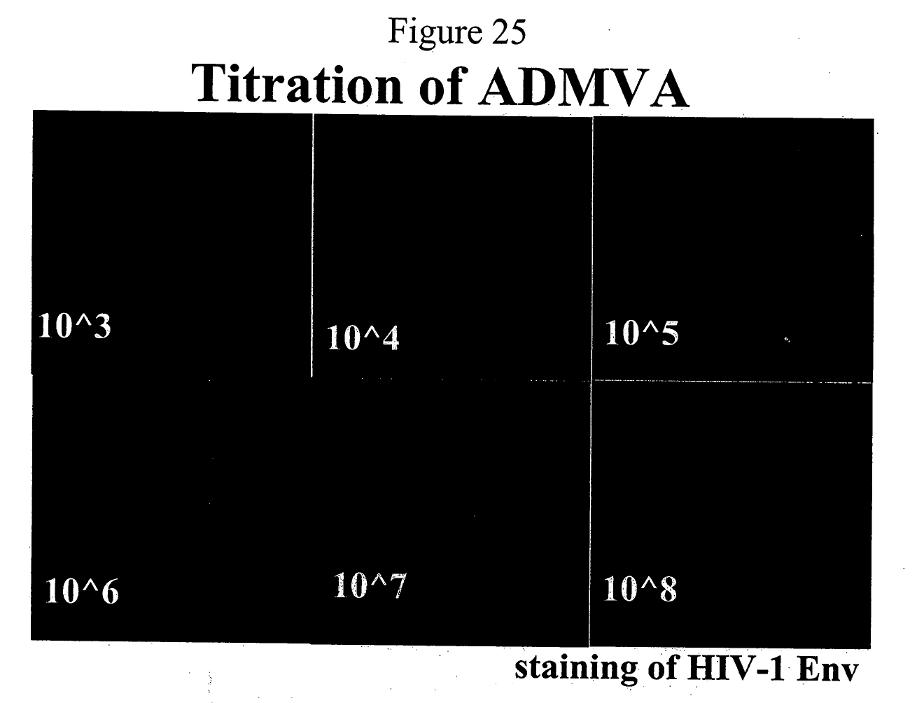

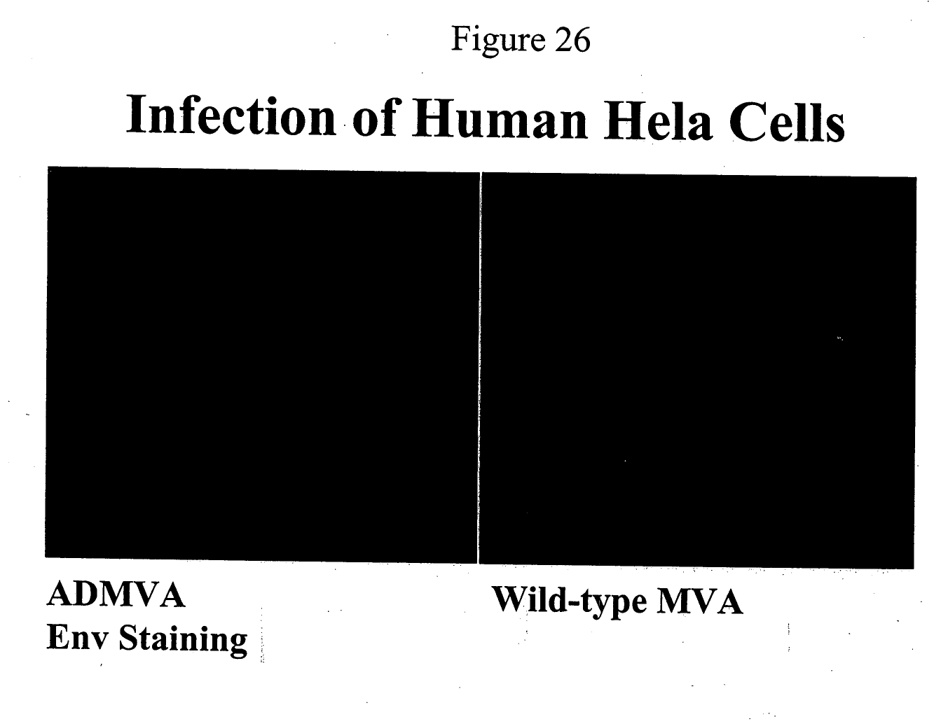

The expression of five HIV-1 gene products in cells post-infected was evaluated with enriched ADMVA. Effective expression of all 5 genes was been confirmed by Western blot analysis (FIG. 24). Additionally, all 5 genes can be amplified from ADMVA genomic DNA. Sequence analysis has confirmed the identity of the inserted genes. The infectivity of ADMVA can reach 10-109 TCID₅₀/mL (FIG. 25), and the virus can be expanded at a 1:10 ratio with ease. ADMVA remained stable after 6 passages in vitro. In addition to chicken embryonic fibroblasts, ADMVA also infected human cells (FIG. 26).

### Example 5

### Preclinical In Vivo Immunogenicity Assessment of ADMVA

Upon completion of the construction and in vitro characterization of ADMVA, we sought to determine the in vivo immunogenicity of this recombinant virus. For the purpose of measuring cell-mediated immune (CMI) responses, in particular, we first chose to use the ELISpot method, which is rapid, reproducible and sensitive for detecting CD8+ and CD4+ T-cell activity. Using the combination peptide pool matrix and splenocytes depleted of CD4+ T cells or CD8+ T cells, CD4 and CD8 epitopes in Env, Gag and Pol, which are presented in the BALB/c mouse were identified. The minimal 9mer epitopes for CD8 (Env and Gag) were predicted by using SYFPEITHI, a database for MHC ligands and peptide motifs (www.unituebingen.de/uni.kxi), purchased from Sigma Genosys (Woodlands, Tex.), and were subsequently confirmed in IFN-γ ELISpot assay.

The peptide sequences are as follows: Env-specific CD4+ T cell epitope (Env 34: VPVWKEAKTTLFCASDAKAY—20mer), and CD8+ T cell epitope (Env T-I: TYNETYNEI—9mer). Gag-specific CD4+ T cell epitope (Gag 26: TSNPPIPVGDIYKRWIILGL—20mer), and CD8+ T cell epitope (Gag A-1: AMQMLKDTI—9mer). Pol-specific CD4+ T cell epitope (Pol 223: TAVQMAVFIHNFKRK—15mer), and CD8+ T cell epitope (Pol 118: VHGVYYDPSKDLIAE—15mer). Tat-specific CD4+ T cell epitope (Tat 12: ISYGRKKRRQRRRAP—15mer).

With the exception of subtype B consensus peptide pools used in the preliminary ELISpot assay, the peptide pools used for Nef and Tat were derived from autologous sequences. Peptides are 15mers overlapping by 11 and were obtained from the IAVI Core Laboratory, Imperial College, London, UK. There are 51 peptides that comprise the entire Nef region. These 51 peptides were divided into two peptide pools. Nef peptide Pool 1 (C.NefP1) contains peptides 1 to 24, and Pool 2 (C.NefP2) contains peptide 25 to 51. The peptide pool for Tat (C.TatP1) contains 23 peptides that comprise full length Tat.

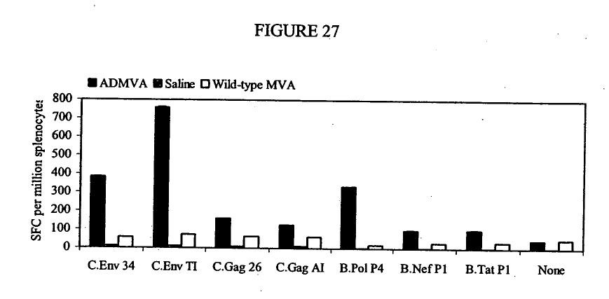

The Research Seed stock of ADMVA was used to immunize 6-8 week-old female BALB/c mice. Specifically, ADMVA vaccine was administered intramuscularly (IM) at weeks 0 and 3. A total of 3 groups of 6 mice each were inoculated as follows: 10⁶ TCID₅₀ of ADMVA, 10⁶ TCID₅₀ of wild-type MVA and saline control. Two weeks after the second injection, the mice were sacrificed. Splenocytes were then pooled from each group and assayed using ELISpot for their ability to secrete interferon-γ (IFN-γ) during re-stimulation in vitro with HIV-1 antigen-specific peptides (NIH AIDS Research and Reference Reagent Program). Results revealed clear immune responses to the five immunogens (gag, pol, env, nef and tat) introduced by the vaccine, yielding approximately 750 spot-forming cells (SFC) per million splenocytes to the strongest epitope, Env TI (FIG. 27). The non-specific background immune response induced by wild-type MVA was approximately 50 SFC/million splenocytes. Predictably, the response to the saline control was less than 10. Of note, the CMI responses to nef and tat were not optimal since peptides from subtype B were used for this assay.

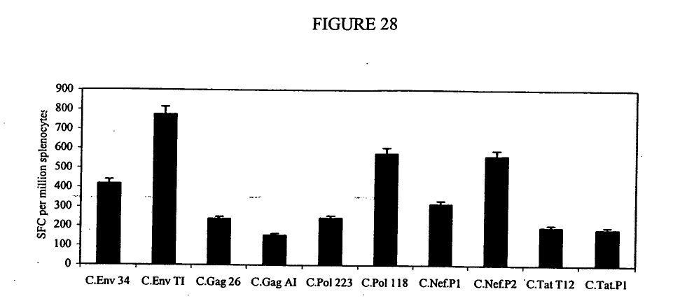

When the experiment was repeated using subtype C-specific homologous peptides for stimulation, the number of SFC increased significantly against nef and tat (FIG. 28). Overall, these CMI responses are directed against at least 9 different epitopes, including those specific for either CD8⁺ (Env TI, Gag AI, Pol 18) or CD4⁺ (Env 34, Gag 26, Pol 223, Tat T12) T-cells. It should be noted, too, that although regulatory proteins nef and tat are co-expressed with structural proteins env, gag and pol in cells infected with ADMVA, the CMI responses to the latter were not eliminated or restricted.

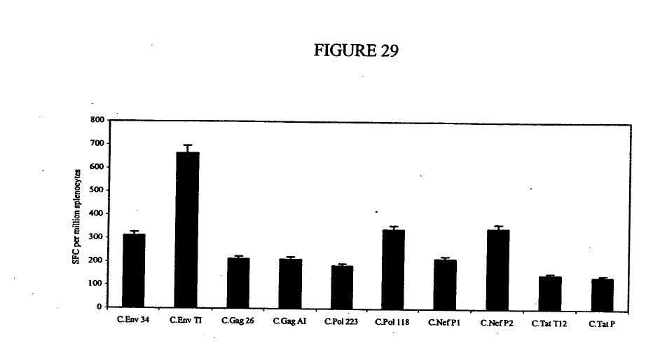

To further determine whether or not the CMI responses elicited in BALB/c mice were strain-specific phenomena, ADMVA was used to immunize 6-8 week-old female B6×B10 mice. Similarly, the vaccine was administered IM at weeks 0 and 3. A total of 6 mice were inoculated with 10⁶ TCID₅₀ of ADMVA. Two weeks after the second injection, the mice were sacrificed. Splenocytes were subjected to ELISpot analysis using homologous subtype-C peptide restimulation (FIG. 29). Based on the resultant SFC counts, comparable CMI responses were found in this mouse species as well. Therefore, the ability of ADMVA to induce broad CMI responses is not limited to a single mouse strain.

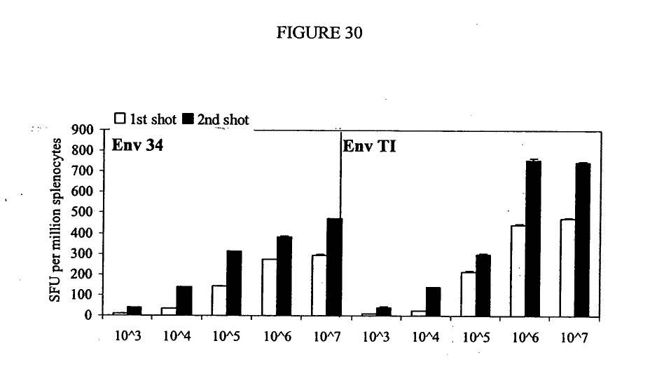

In dose-escalation experiments in mice, we find a clear dose-response effect (FIG. 30). For example, the quantitative ELISpot response to the Env TI CD8 epitope seen for the dose of 10⁶ TCID₅₀ was approximately 20 times higher than that for 10³ TCID₅₀. Moreover, the dose-response trend holds true for all epitopes tested, whether specific for CD4⁺ or CD8⁺ cell-mediated responses. In addition, there is a clear boost effect of MVA after the second immunization for all epitopes tested. Under each dose, all immunized mice tolerated the vaccine very well. ADMVA did not cause any sign of disease or pathogenic effects in immunized mice.

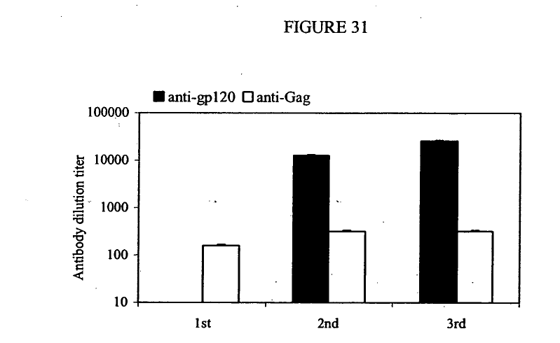

In addition to CMI responses, we also determined the ability of ADMVA to elicit humoral immune responses in mice. Antibody responses were monitored by direct (for Gag) or indirect (for gp120) ELISA. To quantify the antibody response, ELISA with different dilutions of immunized mouse sera was performed. Data from mice immunized with 106 TCID₅₀ ADMVA show that the antibodies against gp120 and Gag were readily detected 2 weeks after the second immunization (FIG. 31). The anti-gp120 antibody titer reached over 1:20,000 after the second immunization.

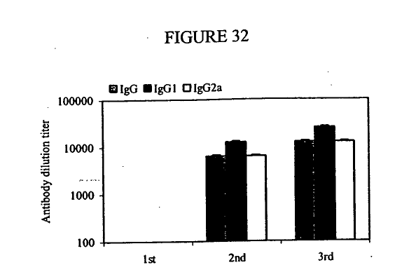

We determined the role of Th1 and Th2 in inducing anti-gp120 antibodies. In mice, Th1 favors IgG2a production whereas Th2 favors IgG1. By measuring the dilution titer of IgG1 and IgG2a, we found similar levels of anti-gp120 antibodies of each subclass (FIG. 32). Therefore, ADMVA elicited rather balanced Th1 and Th2 responses against gp120. We are now in the process of determining the level of neutralizing antibodies in these animals. A preliminary prime-boost experiment was conducted using a 1:1 mixture of ADVAX env/gag plasmid DNA+ADVAX pol/nef-tat plasmid DNA (ADVAX) and ADMVA in BALB/c mice with varying immunization regimens. Each group of four mice received a different immunization regimen as shown below. The mice were sacrificed 2 weeks post boost for the assessment of immune responses. The immunization schedule is shown in Table 2. The results of the prime-boost experiment are summarized in Table 3.

TABLE 2
Prime Boost Schedule

| Group | Prime (Week 0) | Boost (Week 3) |
|---|---|---|
| 1 | ADVAX | ADVAX |
| 2 | ADVAX | ADMVA |
| 3 | ADMVA | ADVAX |
| 4 | ADMVA | ADMVA |

ADVAX DNA dose: 20 μg total DNA per injection (IM)
ADMVA dose: 10⁶ TCID₅₀ per injection (IM)

TABLE 3
CMI responses induced by four different immunization regimens
Antigen Specific IFN-γ SFCs per Million Splenocytes

| Vaccine | | | Env | | Pol | Pol | | | Nef |
|---|---|---|---|---|---|---|---|---|---|
| Regimens | Gag26 | GagA-I | 34 | Env T-I | 223 | VGI | Tat 64 | Tat 60 | Pool |
| ADVAX + | 20 | 50 | 280 | 380 | 70 | 550 | 22 | 20 | 60 |
| ADVAX | | | | | | | | | |
| ADVAX + | 90 | 285 | 260 | 670 | 135 | 600 | 200 | 100 | 245 |
| ADMVA | | | | | | | | | |
| ADMVA + | 40 | 54 | 310 | 585 | 70 | 410 | 40 | 40 | 125 |
| ADVAX | | | | | | | | | |
| ADMVA + | 35 | 40 | 105 | 220 | 42 | 155 | 70 | 55 | 125 |
| ADMVA | | | | | | | | | |

Number of spots was normalized per million splenocytes and averaged for each sample and antigen based on duplicate wells for both the two cell input levels.
Gag 26, Env 34, Pol 223 and Tat 64 contain CD4 epitopes, whereas Gag A-I, Env T-I, Pol VGI and Tat 60 contain CD8 epitopes.
Peptide pool used to assay responses against Nef is based on autologous subtype-C sequences.

Although both CD4+ and CD8+ T cell mediated responses were induced against epitopes of all five MVA-encoded HIV-1 immunogens by all four regimens, ADVAX (DNA) prime plus ADMVA boost induced the strongest overall response.

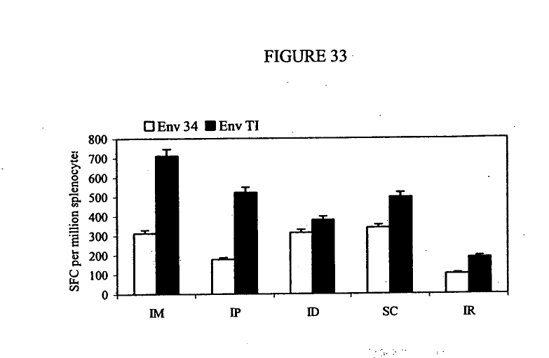

Homologous HIV-1 subtype C peptide-specific CTL responses were induced against all five HIV immunogens in BALB/c mice immunized with the multivalent recombinant ADMVA strain. Furthermore, ADMVA induced CMI responses regardless of the route of administration used in mice, with consistently strong responses observed using the intramuscular route intended for clinical use (FIG. 33). Despite strong MVA specific T-cell responses after a single immunization with ADMVA, mice demonstrated a boosted HIV-specific CMI response after a second immunization with ADMVA administered 3 weeks post priming response. The recombinant MVA vaccine elicited comparable CTL responses in two different strains of mice. Humoral immune responses were also observed when anti-gp120 and anti-gag antibody titers were measured. When the IgG subclasses of the resultant anti-gp120 antibody responses were compared, ADMVA elicited a balanced Th1 and Th2 response in BALB/c mice as shown by the comparable IgG1 and IgG2a env-specific antibody titers.

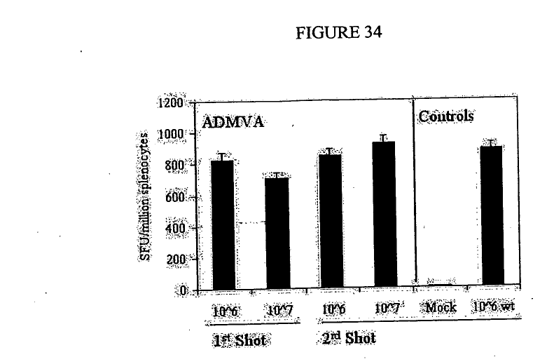

Since people born before 1980 received smallpox vaccination in China, they may have pre-existing immunity against our recombinant MVA vector. For this reason, we sought to determine the magnitude of CMI responses against ADMVA following viral inoculations using a modified ELISpot assay in mice. In this assay, H-2d restricted A20 cells were used as antigen-presenting cells post infection with wild-type MVA at a MOI of 1. The number of SFC against MVA itself reached over 700˜800 two weeks after the first immunization with ADMVA at the doses of 10⁶ TCID₅₀ and 117 TCID₅₀ (FIG. 34). Nonetheless, the same doses of virus were able to boost immune response by approximately 1.5 fold when given a second time (FIG. 29). Since ADMVA will be used as a boosting component for our human trials, our findings support the premise that ADMVA will serve as an effective vaccine booster even in the presence of certain levels of pre-existing immunity to the viral vector.

In an evaluation of several prime-boost regimens using ADVAX plasmid DNA and ADMVA candidate vaccines, the DNA prime+MVA boost regimen induced the strongest CMI responses to peptides representing epitopes expressed by the five HIV-1 transgenes.

### Example 6

### Measurement of Anti-HIV-1 Gag Antibodies in Immunized Animals

A plate was coated (Inanulon-2, Dynex Technologies, Chantilly, Va. or Costar EIA/RIA high binding 96 well plate 9018, Corning Inc., Corning, N.Y.) with 100 μl of Gag protein (0.5 μg/well) overnight at 4° C. in 0.1 M NaHCO₃, pH 9.6. The plate was washed once with 200 μl of phosphate buffered saline (PBS), and blocked with 5% non-fat milk and 0.5% BSA in PBS for 1-2 hour at room temperature. Serially diluted animal serum or controls in the blocking buffer was added and the plate incubated for 1 hour at room temperature. The plate was washed four times with PBS containing 0.05% Tween-20. Alkaline phosphatase-labeled goat anti-mouse IgG (Pharmingen BD) was added; a 1:10,000 dilution per 1 μl conjugate was made in 10 ml blocking buffer, and the plate incubated for 30 minutes at room temperature. The plate was washed four times with AmpliQ wash buffer. The AmpliQ instructions comprised the steps of adding 100 μl per well of freshly made substrate (50 μl solution A combined with 50 μl of solution B) for 15 minutes at room temperature. The reaction was stopped with AmpliQ stop solution and the plate read at 490 nm in a spectrophotometer within 15 min (AmpliQ; DAKO Diagnostics Ltd.).

### Example 7

### Humoral Immune Responses to HIV-1 gp120

Antibodies against HIV-1 gp120 were measured using an indirect ELISA. Costar EIA/RIA high-binding 96-well plates (Corning Inc., Corning, N.Y.) were coated with 100 μl of 5 μg/ml of sheep anti-gp120 antibody to the C terminus of gp120 (International Enzymes, Inc., Fallbrook, Calif.) in 0.1 M NaHCO3 (pH 9.6) overnight at 4° C. The plates were washed with PBS and blocked by adding 5% non-fat milk, 0.5% BSA in PBS for 2 hr at room temperature. Pre-titered subtype C gp120 supernatant was added for 1 hr at room temperature. The plates were then washed four times with PBST. Serially diluted sera from the immunized mice or appropriate controls were added and incubated for 1 hr at room temperature. The plates were washed as above, and 1:10,000 diluted alkaline phosphatase-labeled goat anti-mouse IgG (Pharmingen BD) was added for 30 min at room temperature. The plates were washed three times with AmpliQ wash buffer (DAKO Diagnostics Ltd.), developed using AmpliQ substrate solution, and read at 490 nm within 5 min after the color reaction was terminated with AmpliQ stop solution.

### Example 8

### Mouse IFNγ ELISpot Assay

On day 1, an ELISpot filter plate was pre-coated by adding capture Ab (e.g., mouse IFNγ) at a dilution of 1:50 with coating buffer (e.g., 125 μl in 5 ml of coating buffer). Each well was coated with 100 μl of capture Ab/coating buffer, then covered and incubated at 4° C. overnight. On day 2, cells were harvested, and plated 4× with PBS-Tween. Each well is blocked with R10 (200 μl/well) for 2 hours at 37° C. Cells were added (e.g., 0.5-1.0×10⁶ cells/well) in accordance with the particular plate plan. Subsequently, peptides were added and incubated overnight at 37° C. in a CO₂ incubator. On day 3, plates were washed 5 times with PBS-T, then 100 ul/well of detection Ab was added at a dilution of 1:60 in 1% BSA. Plates were incubated overnight at 4° C. On day 4, plates were washed 4× with PBS-T, then 100 μl/well of SAP added at a 1:60 dilution in 1% BSA. Plates were incubated at room temperature for 2 hours, washed 4× with PBS-T, and then washed 1× with double distilled H₂0. Each well then contained 100 μl of substrate, and then incubated in the dark at RT for about 15 minutes or until fully developed. The plates were washed with tap water, dried thoroughly and visualized for immunoreactive spots.

Having thus described in detail preferred embodiments of the present invention, it is to be understood that the invention defined by the appended claims is not to be limited to particular details set forth in the above description, as many apparent variations thereof are possible without departing from the spirit or scope of the present invention. Modifications and variations of the method and apparatuses described herein will be obvious to those skilled in the art, and are intended to be encompassed by the following claims.

### REFERENCES

AIDS Epidemic Update, December 2002, Joint UNAIDS/WHO Aiken, C., Konner, J., Landau, N. R., Lenburg, M. E., Trono, D.: “Nef induces CD4 endocytosis: requirement for a critical dileucine motif in the membrane-proximal CD4 cytoplasmic domain.” (1994) Cell 76(5): 853-64.
Allen, T. M., Vogel, T. U., Fuller, D. H., Mothe, B. R., Steffen, S., Boyson, J. E., Shipley, T., Fuller, J., Hanke, T., Sette, A., Altman, J. D., Moss, B., McMichael, A. J., and Watkins D. I.: “Induction of AIDS virus-specific CTL activity in fresh, unstimulated peripheral blood lymphocytes from rhesus macaques vaccinated with a DNA prime/modified vaccinia virus Ankara boost regimen”. (2000) J. Immunol. 164(9): 4968-78.
Altenburger, W., U.S. Pat. No. 5,185,146
Altenburger, W., Suter, C. P., and Altenburger, J.: “Partial deletion of the human host range gene in the attenuated vaccinia virus MVA”. (1989) Arch Virol. 105(1-2): 15-27.
Amara, R. R., Villinger, F., Altman, J. D., Lydy, S. L., O'Neil, S. P., Staprans, S. I., Montefiori, D. C., Xu, Y., Herndon, J. G., Wyatt, L. S., Candido, M. A., Kozyr, N. L., Earl, P. L., Smith, J. M., Ma, H. L., Grimm, B. D., Hulsey, M. L., Miller, J., McClure, H. M., McNicholl, J. M., Moss, B., Robinson, H. L.: “Control of a mucosal challenge and prevention of AIDS by a multiprotein DNA/MVA vaccine”. (2001) Science 292(5514): 69-74.
Barnett, S. W., Lu, S. Srivastava, I. Cherpelis, S. Gettie, A. Blanchard, J. Wang, S. Mboudjeka, I. Leung, L. Lian, Y. Fong, A. Buckner, C. Ly, A. Hilt, S. Ulmer, J. Wild, C. T. Mascola, J. R. and Stamatatos, L.: “The ability of an oligomeric human immunodeficiency virus type 1 (HIV-1) envelope antigen to elicit neutralizing antibodies against primary HIV-1 isolates is improved following partial deletion of the second hypervariable region” (2001) J. Virol. 75: 5526-40.
Barouch, D. H., Santra, S., Schmitz, J. E., Kuroda, M. J., Fu, T. M., Wagner, W., Bilska, M., Craiu, A., Zheng, X. X., Krivulka, G. R., Beaudry, K., Lifton, M. A., Nickerson, C. E., Trigona, W. L., Punt, K., Freed, D. C., Guan, L., Dubey, S., Casimiro, D., Simon, A., Davies, M. E., Chastain, M., Strom, T. B., Gelman, R. S., Montefiori, D. C., Lewis, M. G., Emini, E. A., Shiver, J. W., and Letvin, N. L.: “Control of viremia and prevention of clinical AIDS in rhesus monkeys by cytokine-augmented DNA vaccination”. (2000) Science 290(5491): 486-92.
Barouch, D. H., Santra, S., Kuroda, M. J., Schmitz, J. E., Plishka, R., Buckler-White, A., Gaitan, A. E., Zin, R., Nam, J. H., Wyatt, L. S., Lifton, M. A., Nickerson, C. E., Moss, B., Montefiori, D. C., Hirsch, V. M., and Letvin, N. L.: “Reduction of simian-human immunodeficiency virus 89.6P viremia in rhesus monkeys by recombinant modified vaccinia virus Ankara vaccination”. (2001) J. Virol. 75(11): 5151-8.
Bender, B. S., Rowe, C. A., Taylor, S. F., Wyatt, L. S., Moss, B., Small, P. A. Jr.: “Oral immunization with a replication-deficient recombinant vaccinia virus protects mice against influenza” (1996) J. Virol. 70(9): 6418-24.
Beyrer, C., Razak, M. H., Lisam, K., Chen, J., Lui, W., and Yu, X. F.: “Overland heroin trafficking routes and HIV-1 spread in South and Southeast Asia” (2000) AIDS 14(1): 75-83.
Blanchard, T. J., Alcami, A., Andrea, P., and Smith, G. L.: “Modified vaccinia virus Ankara undergoes limited replication in human cells and lacks several immunomodulatory proteins: implications for use as a human vaccine”. (1998) J Gen Virol. 79 (Pt 5): 1159-67
Buttner, M., Czerny, C. P., Lehner, K. H., and Wertz, K.: “Interferon induction in peripheral blood mononuclear leukocytes of man and farm animals by poxvirus vector candidates and some poxvirus constructs”. (1995) Vet Immunol Immunopathol. 46(3-4): 237-50.
Chakrabarti, S., Brechling, K., and Moss, B.: “Vaccinia virus expression vector: coexpression of beta-galactosidase provides visual screening of recombinant virus plaques” (1985) Mol. Cell. Biol. 5(12): 3403-9.
Calarota, S. A., Leandersson, A. C., Bratt, G., Hinkula, J., Klinman, D. M., Weinhold, K. J., Sandstrom, E., and Wahren, B.: “Immune responses in asymptomatic HIV-1-infected patients after HIV-DNA immunization followed by highly active antiretroviral treatment”. (1999) J Immunol. 163(4): 2330-8.
Carvalho, L. H., Hafalla, J. C., and Zavala F.: “ELISPOT assay to measure antigen-specific-murine CD8(+) T cell responses”. (2001) J Immunol Methods. 252(1-2): 207-18.
Chao, S. F., Chan, V. L., Juranka, P., Kaplan, A. H., Swanstrom, R., Hutchison, C. A. 3rd.: “Mutational sensitivity patterns define critical residues in the palm subdomain of the reverse transcriptase of human immunodeficiency virus type 1”. (1995) Nucleic Acids Res. 23(5): 803-10.
Cherpelis, S., Jin, X. Gettlie, A. Ho, D. D., Barnett, S. W. Shrivastava, I. and Stamatatos, L.; “DNA-immunization with a V2 deleted HIV-1 envelope elicits protective antibodies in macaques”. (2001) Immunol Lett 79: 47-55.
Cherpelis, S., Shrivastava, I. Gettie, A. Jin, X. Ho, D. D. Barnett, S. W. and Stamatatos L.: “DNA vaccination with the human immunodeficiency virus type I SF162DeltaV2 envelope elicits immune responses that offer partial protection from simian/human immunodeficiency virus infection to CD8(+) T-cell-depleted rhesus macaques.” (2001) J Virol 75: 1547-50.
Coffin, J M Hughes, S M Varmus, H E “Retroviruses” (1997) Cold Spring Harbor Laboratory Press Eds, pp. 758-763.
Collins, K. L., Chen, B. K., Kalams, S. A., Walker, B. D., and Baltimore, D.: “HIV-1 Nef protein protects infected primary cells against killing by cytotoxic T lymphocytes.” (1998) Nature 391(6665): 397-401.
Collins, K. L., and Baltimore, D.: “HIV's evasion of the cellular immune response”. (1999) Immunol Rev. 168: 65-74.
Donnelly, J. J., Barnett, S. W. Dorenbaum, A. and Stamatatos, L.: “Envelope-based HIV vaccines”. (2002) Science 297: 1277-8; discussion 1277-8.
Dorner, F., U.S. Pat. No. 6,265,183
Dorner, F., U.S. Pat. No. 5,445,953
Falkner, F. G., U.S. Pat. No. 5,766,882
Friedmann, T., U.S. Pat. No. 6,451,304
Friedmann, T. Ed., The development of human gene therapy (1999) Cold Spring Harbor Press
Garber, M. E., Jones, K. A.: “HIV-1 Tat: coping with negative elongation factors”. (1999) Curr Opin Immunol. 11(4): 460-5.
Girard M.: “Prospects for an AIDS vaccine”. (1990) Cancer Detect Prev. 14(3):411-3.
Goldstein, G.: “HIV-1 Tat protein as a potential AIDS vaccine”. (1996) Nat. Med. 2(9):960-4.
Garber, M. E., and Jones, K. A.: ‘HIV-1 Tat: coping with negative elongation factors’. (1999) Curr Opin Immunol. 11(4): 460-5.
Haddad, D., Liljeqvist, S., Stahl, S., Andersson, I., Perlmann, P., Berzins, K., and Ahlborg, N.: “Comparative study of DNA-based immunization vectors: effect of secretion signals on the antibody responses in mice”. (1997) FEMS Immunol Med Microbiol. 18(3): 193-202.
Hanke, T., Blanchard, T. J., Schneider, J., Ogg, G. S., Tan, R., Becker, M., Gilbert, S. C., Hill, A. V., Smith, G. L., and McMichael, A.: “Immunogenicities of intravenous and intramuscular administrations of modified vaccinia virus Ankara-based multi-CTL epitope vaccine for human immunodeficiency virus type 1 in mice.” (1998) J Gen Virol. 79 (Pt 1): 83-90.
Hanke, T., Neumann, V. C., Blanchard, T. J., Sweeney, P., Hill, A. V., Smith, G. L., and McMichael, A.: “Effective induction of HIV-specific CTL by multi-epitope using gene gun in a combined vaccination regime”. (1999) Vaccine. 17(6): 589-96.
Hanke, T., Samuel, R. V., Blanchard, T. J., Neumann, V. C., Allen, T. M., Boyson, J. E., Sharpe, S. A., Cook, N., Smith, G. L., Watkins, D. I., Cranage, M. P., and McMichael, A. J.: “Effective induction of simian immunodeficiency virus-specific cytotoxic T lymphocytes in macaques by using a multiepitope gene and DNA prime-modified vaccinia virus Ankara boost vaccination regimen”. (1999) J. Virol. 73(9): 7524-32.
Haynes, B. F.: “HIV vaccines: where we are and where we are going” (1996) Lancet. 348(9032): 933-7.
Hirsch, V. M., Fuerst, T. R., Sutter, G., Carroll, M. W., Yang, L. C., Goldstein, S., Piatak, M. Jr, Elkins, W. R., Alvord, W. G., Montefiori, D. C., Moss, B., and Lifson, J. D.: “Patterns of viral replication correlate with outcome in simian immunodeficiency virus (SIV)-infected macaques: effect of prior immunization with a trivalent SIV vaccine in modified vaccinia virus Ankara”. (1996) J. Virol. 70(6): 3741-52.
Hu, S. L., Kosowski, S. G., and Dahrymple, J. M.: “Expression of AIDS virus envelope gene in recombinant vaccinia viruses”. (1986) Nature 320: 537-40.
Jin, X., Bauer, D. E., Tuttleton, S. E., Lewin, S., Gettie, A., Blanchard, J., Irwin, C. E., Safrit, J. T., Mittler, J., Weinberger, L., Kostrikis, L. G., Zhang, L., Perelson, A. S., and Ho, D. D.: “Dramatic rise in plasma viremia after CD8(+) T cell depletion in simian immunodeficiency virus-infected macaques.” (1999) J Exp Med. 189(6): 991-8.
Kimpton, J., and Emerman, M.: ‘Detection of replication-competent and pseudotyped human immunodeficiency virus with a sensitive cell line on the basis of activation of an integrated beta-galactosidase gene”. (1992) J. Virol. 66(4): 2232-9.
Kingsman, A. J. WO 99/41397
Kotsopoulou, E., Kim, V. N., Kingsman, A. J., Kingsman, S. M., and Mitrophanous, K. A.: “A Rev-independent human immunodeficiency virus type 1 (HIV-1)-based vector that exploits a codon-optimized HIV-1 gag-pol gene” (2000) J. Virol. 74(10):4839-5.2.
Lewis, P., Hensel, M., Emerman, M.: “Human immunodeficiency virus infection of cells arrested in the cell cycle” (1992) EMBO J. 11(8): 3053-8.
Lewis, P. F., and Emerman, M.: “Passage through mitosis is required for oncoretroviruses but not for the human immunodeficiency virus”. (1994) J. Virol. 68(1): 510-6.
Li, Z., Howard, A., Kelley, C., Delogu, G., Collins, F., and Morris, S.: “Immunogenicity of DNA vaccines expressing tuberculosis proteins fused to tissue plasminogen activator signal sequences”. (1999) Infect Immun. 67(9): 4780-6.
Lieberman, H. A. Pharmaceutical dosage forms (1991) Marcel Dekker Vol. 1-3
Allen, L. V., Lachman, L., Schwartz, J. B., (1999) The Art, Science, and Technology of Pharmaceutical Compounding.
Loeb, D. D., Swanstrom, R., Everitt, L., Manchester, M., Stamper, S. E., and
Hutchison, C. A. 3rd.: “Complete mutagenesis of the HIV-1 protease”. (1989) Nature 340(6232): 397-400.
Ly, A., and Stamatatos, L.: “V2 loop glycosylation of the human immunodeficiency virus type 1 SF162 envelope facilitates interaction of this protein with CD4 and CCR5 receptors and protects the virus from neutralization by anti-V3 loop and anti-CD4 binding site antibodies. (2001) J Virol 74: 6769-76.
Martinez-Salas, E.: “Internal ribosome entry site biology and its use in expression vectors”. (1999) Curr Opin Biotechnol. 10(5): 458-64.
Mayr, A., and Danner, K.: “Vaccination against pox diseases under immunosuppressive conditions”. (1978) Dev Biol Stand 41: 225-34.
Mayr, A., Stickl, H., Muller, H. K., Danner, K., and Singer, H.: “The smallpox vaccination strain MVA: marker, genetic structure, experience gained with the parenteral vaccination and behavior in organisms with a debilitated defense mechanism” (1978) Zentralbl Bakteriol [B]. 167(5-6): 375-90
McMichael, A. J., and Rowland-Jones, S. L.: “Cellular immune responses to HIV”. (2001) Nature. 410(6831):980-7.
Men, R., Wyatt, L. Tokimatsu, I. Arakaki, S. Shameem, G. Elkins, R. Chanock, R
Moss, B. and Lai, C. J.: “Immunization of rhesus monkeys with a recombinant of modified vaccinia virus Ankara expressing a truncated envelope glycoprotein of dengue type 2 virus induced resistance to dengue type 2 virus challenge”. (2000) Vaccine 18: 3113-22.
Merchlinsky, M., and Moss, B.: “Introduction of foreign DNA into the vaccinia virus genome by in vitro ligation: recombination-independent selectable cloning vectors”. (1992) Virology 190(1): 522-6.
Meyer, H., Sutter, G., and Mayr, A.: “Mapping of deletions in the genome of the highly attenuated vaccinia virus MVA and their influence on virulence”. (1991) J Gen Virol. 72 (Pt 5): 1031-8.
Moss, B., U.S. Pat. No. 5,866,383
Moss, B., Carroll, M. W., Wyatt, L. S., Bennink, J. R., Hirsch, V. M., Goldstein, S., Elkins, W. R., Fuerst, T. R., Lifson, J. D., Piatak, M., Restifo, N. P., Overwijk, W., Chamberlain, R., Rosenberg, S. A., and Sutter, G.: “Host range restricted, non-replicating vaccinia virus vectors as vaccine candidates”. (1996) Adv Exp Med. Biol. 397: 7-13.
Moss B.: “Genetically engineered poxviruses for recombinant gene expression, vaccination, and safety.” (1996) Proc Natl Acad Sci USA. 93(21): 11341-8.
Najjar, S. M., and Lewis, R. E.: ‘Persistent expression of foreign genes in cultured hepatocytes: expression vectors” (1999) Gene. 230(1): 41-5.
Nishimura, Y., Kamei, A., Uno-Furuta, S., Tamaki, S., Kim, G., Adachi, Y., Kuribayashi, K., Matsuura, Y., Miyamura, T., Yasutomi, Y.: “A single immunization with a plasmid encoding hepatitis C virus (HCV) structural proteins under the elongation factor 1-alpha promoter elicits HCV-specific cytotoxic T-lymphocytes (CTL)”. (1999) Vaccine 18(7-8): 675-80.
Novitsky, V., Rybak, N., McLane, M. F., Gilbert, P., Chigwedere, P., Klein, I., Gaolekwe, S., Chang, S. Y., Peter, T., Thior, I., Ndung'u, T., Vannberg, F., Foley, B. T., Marlink, R., Lee, T. H., and Essex, M.: “Identification of human immunodeficiency virus type 1 subtype C Gag-, Tat-, Rev-, and Nef-specific elispot-based cytotoxic T-lymphocyte responses for AIDS vaccine design”. (2001) J. Virol. 75(19): 9210-28.
Ogg, G. S., Jin, X., Bonhoeffer, S., Dunbar, P. R., Nowak, M. A., Monard, S., Segal, J. P., Cao, Y., Rowland-Jones, S. L., Cerundolo, V., Hurley, A., Markowitz, M., Ho, D. D., Nixon, D. F., and McMichael, A. J.: “Quantitation of HIV-1-specific cytotoxic T lymphocytes and plasma load of viral RNA” (1998) Science 279(5359): 2103-6.
Ourmanov, I., Brown, C. R. Moss, B. Carroll, M. Wyatt, L. Pletneva, L. Goldstein, S. Venzon, D. and Hirsch, V. M.: “Comparative efficacy of recombinant modified vaccinia virus Ankara expressing simian immunodeficiency virus (SIV) Gag-Pol and/or Env in macaques challenged with pathogenic SIV”. (2000) J Virol 74: 2740-51.
Paoletti, E., U.S. Pat. No. 5,972,708
Paoletti, E., U.S. Pat. No. 6,340,462
Paoletti, E., U.S. Pat. No. 5,972,597
Paoletti, E., U.S. Pat. No. 5,225,336
Paoletti, E., U.S. Pat. No. 5,204,243
Paoletti, E., U.S. Pat. No. 5,155,020
Paoletti, E., U.S. Pat. No. 5,110,587
Paoletti, E., U.S. Pat. No. 4,769,330
Paoletti, E., U.S. Pat. No. 4,722,848
Paoletti, E., U.S. Pat. No. 4,603,112
Paoletti, E., U.S. Pat. No. 6,596,279
Paoletti, E., U.S. Pat. No. 5,762,938
Paoletti, E., U.S. Pat. No. 5,453,364
Paoletti, E., U.S. Pat. No. 5,378,457
Paoletti, E., U.S. Pat. No. 5,364,773
Paoletti, E., U.S. Pat. No. 5,863,542
Paoletti, E., U.S. Pat. No. 5,766,598
Paoletti, E., U.S. Pat. No. 5,756,103
Paoletti, E., U.S. Pat. No. 5,494,807
Paoletti, E., U.S. Pat. No. 5,364,773
Paoletti, E., U.S. Pat. No. 4,769,330
Paoletti, E., U.S. Pat. No. 5,155,020
Paoletti, E., U.S. Pat. No. 5,204,243
Paoletti E.: “Applications of poxvirus vectors to vaccination: an update”. (1996) Proc Natl Acad Sci USA. 93(21): 11349-53.
Pfleiderer, M., Falkner, F. G., and Dørner, F.: “A novel vaccinia virus expression system allowing construction of recombinants without the need for selection markers, plasmids and bacterial hosts”. (1995) J Gen Virol. 76 (Pt 12): 2957-62.
Pickar, G. D. Dosage Calculations (1999) Delmar Learning, 6^{th} Edition
Piyasirisilp, S., McCutchan, F. E., Carr, J. K., Sanders-Buell, E., Liu, W., Chen, J., Wagner, R., Wolf, H., Shao, Y, Lai, S., Beyrer, C., and Yu, X. F.: “A recent outbreak of human immunodeficiency virus type 1 infection in southern China was initiated by two highly homogeneous geographically separated strains, circulating recombinant form AE and a novel BC recombinant” (2000) J. Virol. 74(23): 11286-95.
Qiu, J. T., Liu, B., Tian, C., Paviakis, G. N., and Yu, X. F.: “Enhancement of primary and secondary cellular immune responses against human immunodeficiency virus type 1 gag by using DNA expression vectors that target Gag antigen to the secretory pathway”. (2000) J. Virol. 74(13): 5997-6005 Remington's Pharmaceutical Sciences, 17^{th} Ed., (1989) Mack Publishing
Robinson, H. L., Montefiori, D. C., Johnson, R. P., Manson, K. H., Kalish, M. L., Lifson, J. D., Rizvi, T. A., Lu, S., Hu, S. L., Mazzara, G. P., Panicali, D. L., Herndon, J. G., Glickman, R., Candido, M. A., Lydy, S. L., Wyand, M. S., and McClure, H. M.: “Neutralizing antibody-independent containment of immunodeficiency virus challenges by DNA priming and recombinant pox virus booster immunizations”. (1999) Nat. Med. 5(5): 526-34.
Sambrook, V., Fritsch, E. F., and Maniatis, T., Molecular Cloning: A Laboratory Manual 2^{nd} Ed. (1989) Cold Spring Harbor Laboratory Press
Schmitz, J. E., Kuroda, M. J., Santra, S., Sasseville, V. G., Simon, M. A., Lifton, M. A., Racz, P., Tenner-Racz, K., Dalesandro, M., Scallon, B. J., Ghrayeb, J., Forman, M. A., Montefiori, D. C., Rieber, E. P., Letvin, N. L., and Reimann, K. A.: “Control of viremia in simian immunodeficiency virus infection by CD8+ lymphocytes”. (1999) Science 283(5403): 857-60.
Schneider, R., Campbell, M., Nasioulas, G., Felber, B. K., and Pavlakis, G. N.: “Inactivation of the human immunodeficiency virus type 1 inhibitory elements allows Rev-independent expression of Gag and Gag/protease and particle formation”. (1997) J. Virol. 71(7): 4892-903.
Sodroski, J. G., U.S. Pat. No. 5,665,577
Spira, S., Wainberg, M. A., Loemba, H., Turner, D., and Brenner, B. G.: “Impact of clade diversity on HIV-1 virulence, antiretroviral drug sensitivity and drug resistance”. (2003) J Antimicrob Chemother. 51(2): 229-40.
Stamatatos, L., Lim, M., and Cheng Mayer, C.: “Generation and structural analysis of soluble oligomeric gp140 envelope proteins derived from neutralization-resistant and neutralization-susceptible primary HIV type 1 isolates.” (2000) AIDS Res Hum Retroviruses 16: 981-94.
Stickl, H., Hochstein-Mintzel, V. Mayr, A. Huber, H. C. Schafer, H. and Holzner, A.: “MVA vaccination against smallpox: clinical tests with an attenuated live vaccinia virus strain (MVA) (author's transl)”. (1974) Dtsch Med Wochenschr 99: 2386-92.
Sutter, G., and Moss, B.: “Nonreplicating vaccinia vector efficiently expresses recombinant genes”. (1992) Proc Natl Acad Sci USA. 89(22): 10847-51.
Sutter, G., Wyatt, L. S. Foley, P. L. Bennink, J. R. and Moss, B.: “A recombinant vector derived from the host range-restricted and highly attenuated MVA strain of vaccinia virus stimulates protective immunity in mice to influenza virus”. (1994) Vaccine 12: 1032-40.
Takahashi, H., Cohen, J. Hosmalin, A. Cease, K. B. Houghten, R. Cornette, J. L. DeLisi, C. Moss, B. Germain, R. N. and Berzofsky, J. A.: “An immunodominant epitope of the human immunodeficiency virus envelope glycoprotein gp 160 recognized by class I major histocompatibility complex molecule-restricted murine cytotoxic T lymphocytes.” (1988) Proc Natl Acad Sci USA 85: 3105-9.
Tang, D. C., Devit, M., Johnston, S. A.: “Genetic Immunization is a Simple Method for Eliciting an Immune Response” (1992) Nature 356: 152-154
Tobery, T. W., Wang, S., Wang, X. M., Neeper, M. P., Jansen, K. U., McClements, W. L., and Caulfield, M. J.: “A simple and efficient method for the monitoring of antigen-specific T cell responses using peptide pool arrays in a modified ELISpot assay”. (2001) J Immunol Methods. 254(1-2): 59-66.
Wakefield, J. K., Jablonski, S. A., and Morrow, C. D.: “In vitro enzymatic activity of human immunodeficiency virus type 1 reverse transcriptase mutants in the highly conserved YMDD amino acid motif correlates with the infectious potential of the proviral genome”. (1992) J. Virol. 66(11): 6806-12.
Walker, S., Sofia, M. J., Kakarla, R., Kogan, N. A., Wierichs, L., Longley, C. B., Bruker, K., Axelrod, H. R., Midha, S., Babu, S., and Kahne, D.: “Cationic facial amphiphiles: a promising class of transfection agents.” (1996) Proc Natl Acad Sci USA. 93(4): 1585-90.
Wallich, R., Siebers, A., Jahraus, O., Brenner, C., Stehle, T., and Simon, M. M.: “DNA vaccines expressing a fusion product of outer surface proteins A and C from Borrelia burgdorferi induce protective antibodies suitable for prophylaxis but Not for resolution of Lyme disease”. (2001) Infect Immun. 69(4): 2130-6.
Weiss, R., Dumberger, J., Mostbock, S., Scheiblhofer, S., Hartl, A., Breitenbach, M., Strasser, P., Domer, F., Livey, I., Crowe, B., and Thathamer, J.: “Improvement of the immune response against plasmid DNA encoding OspC of Borrelia by an ER-targeting leader sequence.” (1999) Vaccine 18(9-10): 815-24.
Williams, S. G., Cranenburgh, R. M., Weiss, A. M., Wrighton, C. J., Sherratt, D. J., and Hanak, J. A.: “Repressor titration: a novel system for selection and stable maintenance of recombinant plasmids”. (1998) Nucleic Acids Res. 26(9): 2120-4.
Yu, X. F., Liu, W., Chen, J., Kong, W., Liu, B., Yang, J., McCutchan, F., Piyasirisilp, S., Lai, S.: “Rapid dissemination of a novel B/C recombinant HIV-1 among injection drug users in southern China” (2001) AIDS 15(4): 523-5.

1. A nucleic acid vector comprising at least one HIV sequence operably linked to a promoter, wherein the HIV sequence(s) encode a protein(s) that does not assemble into viral particles.

2. A nucleic acid vector comprising at least two HIV sequences, wherein the HIV sequences are each operably linked to separate promoters and wherein the HIV sequences encode proteins that do not assemble into viral particles.

3. The nucleic acid vector of claim 1, wherein said HIV sequences are selected from the group consisting of env, gag, pol, tat, rev, nef, vif, vpr, vpu, vpx, muteins, fusions and portions thereof.

4. The nucleic acid vector of claim 1, wherein the promoter(s) is a heterologous promoters.

5. The nucleic acid vector of claim 4, wherein the heterologous promoter(s) is selected from the group consisting of prokaryotic promoters, eukaryotic promoters and viral promoters.

6. The nucleic acid vector of claim 2, wherein the promoters are different promoters.

7. The nucleic acid vector of claim 1, further comprising a transcriptional terminator downstream of said HIV sequences.

8. The nucleic acid vector of claim 7, wherein the transcriptional terminator is a polyadenylation signal.

9. The nucleic acid vector of claim 8, wherein the transcriptional terminator is a polyadenylation signal selected from the group consisting of the bovine growth hormone (bGH) polyadenylation signal, the SV40 polyadenylation signal, and the vaccinia polyadenylation signal.

10. The nucleic acid vector of claim 1, wherein at least one HIV sequence is operably linked to a heterologous leader sequence.

11. The nucleic acid vector of claim 10, wherein the leader sequence is selected from the group consisting of the tissue plasminogen activator (tPA) leader sequence, the α-factor leader sequence, the pre-proinsulin leader sequence, the invertase leader sequence, the immunoglobulin A leader sequence, the ovalbumin leader sequence, and the β-globin leader sequence.

12. The nucleic acid vector of claim 10, wherein viral particles are not assembled as a result of tPA-gag.

13. The nucleic acid vector of claim 1, wherein said HUV sequence(s) is selected from the group consisting of SEQ ID NO:7, SEQ ID NO:9, SEQ ID NO:11, SEQ ID NO:13, SEQ ID NO:17 and SEQ ID NO:19.

14. The nucleic acid vector of claim 1, further comprising an origin of replication.

15. The nucleic acid vector of claim 1, further comprising a selectable marker gene.

16. A nucleic acid vector comprising at least two HIV sequences, wherein the HIV sequences:
a) are each operably linked to separate promoters,
b) encode proteins that do not assemble into viral particles, and
c) at least one HIV sequence is operably linked to a heterologous leader sequence, the vector optionally further comprising a downstream transcriptional terminator, an origin of replication and a selectable marker gene.

17. The nucleic acid vector of claim 16, wherein the HIV sequences encode for tPA-env and tPA-gag.

18. The nucleic acid vector of claim 16, wherein the HIV sequences are SEQ ID NO:7 and SEQ ID NO:9.

19. The nucleic acid vector of claim 16, wherein the HIV sequences encode for tPA-pol and tPA-nef-tat.

20. The nucleic acid vector of claim 16, wherein the HIV sequences are SEQ ID NO: 11 and SEQ ID NO: 13.

21. The nucleic acid vector of claim 1, wherein the vector is a viral vector.

22. The nucleic acid vector of claim 21, wherein the viral vector is selected from the group consisting of modified vaccine Ankara (MVA), ALVAC, NYVAC. 1, and NYVAC.2.

23. The nucleic acid vector of claim 22, wherein the vector is a modified vaccinia Ankara (MVA) vector.

24. The nucleic acid vector of claim 22, wherein the promoter is selected from the group consisting of the poxyiral 7.5K promoter, the poxyiral 40K promoter, the poxyiral H5 promoter, the poxyiral 11K promoter, the poxyiral I3 promoter, the poxyiral synthetic (SYN) promoter, and the poxyiral synthetic early/late promoter.

25. A nucleic acid vector, wherein the vector is a modified vaccinia Ankara (MVA) vector comprising:
a) at least two HIV sequences inserted into deletion site III of the MVA genome; and
b) at least one HIV sequence inserted into deletion site II of the MVA genome; and
wherein each HIV sequence is operably linked to a separate promoter and wherein the HIV sequences encode proteins that do not assemble into viral particles.

26. The nucleic acid vector of claim 25, wherein the HIV sequences are selected from the group consisting of env, gag, pol, tat, rev, nef, vif, vpr, vpu, vpx, muteins, fusions, and portions thereof.

27. The nucleic acid vector of claim 25, wherein the promoters are selected from the group consisting of the poxyiral 7.5K promoter, the poxyiral 40K promoter, the poxyiral H5 promoter, the poxyiral 11K promoter, the poxyiral I3 promoter, the poxyiral synthetic (SYN) promoter, and the poxyiral synthetic early/late promoter.

28. The nucleic acid vector of claim 25, wherein the HIV sequences further comprise a heterologous leader sequence.

29. The nucleic acid vector of claim 25, wherein the heterologous leader sequence is selected from the group consisting of the tissue plasminogen activator (tPA) leader sequence, the α-factor leader sequence, the pre-proinsulin leader sequence, the invertase leader sequence, the immunoglobulin A leader sequence, the ovalbumin leader sequence, and the α-globin leader sequence.

30. The nucleic acid vector of claim 25, wherein tPA-delta V2 env and tPA-gag-pol are inserted into deletion site III of MVA and tPA-nef-tat is inserted into deletion site II of MVA.

31. The nucleic acid vector of claim 25, wherein SEQ ID NO:17 and SEQ ID NO:19 are inserted into deletion site III of MVA and SEQ ID NO:21 is inserted into deletion site II of MVA.

32. A composition comprising at least one of the nucleic acid vectors as in claim 1.

33. The composition of claim 32, wherein tPA-env and tPA-gag are on a first nucleic acid vector and tPA-pol and tPA-nef-tat are on a second nucleic acid vector.

34. The composition of claim 32, wherein SEQ ID NO:7 and SEQ ID NO:9 are on a first nucleic acid vector and SEQ ID NO:11 and SEQ ID NO:13 are on a second nucleic acid vector.

35. The composition of claim 32, wherein at least one vector is a viral vector.

36. The composition of claim 35, where the viral vector is selected from the group consisting of modified vaccine Ankara (MVA), ALVAC, NYVAC. 1, and NYVAC.2.

37. The composition of claim 36, wherein the viral vector is a MVA vector.

38. The composition of claim 37, wherein:
a) tPA-env and tPA-gag are on a first nucleic acid vector and tPA-pol and tPA-nef-tat are on a second nucleic acid vector, and
b) tPA-delta V2 env and tPA-gag-pol are inserted into deletion site III of MVA and tPA-nef-tat is inserted into deletion site II of MVA.

39. The composition of claim 37, wherein:
a) SEQ ID NO:7 and SEQ ID NO:9 are on a first nucleic acid vector and SEQ ID NO:11 and SEQ ID NO:13 are on a second nucleic acid vector, and
b) SEQ ID NO:17 and SEQ ID NO:19 are inserted into deletion site III of MVA and SEQ ID NO:21 is inserted into deletion site II of MVA.

40. A pharmaceutical composition comprising a nucleic acid vector as in claim 1 and a pharmaceutically acceptable carrier, adjuvant or excipient.

41. A pharmaceutical composition comprising a composition as in claim 32 and a pharmaceutically acceptable carrier, adjuvant or excipient.

42. A method of eliciting an immune response in a subject susceptible to an HIV-related disease or condition comprising administering a nucleic acid vector as claim 1 to the subject, thereby eliciting an immune response against HIV.

43. A method of eliciting an immune response in a subject susceptible to an HIV-related disease or condition comprising administering a composition as in claim 32 to the subject, thereby eliciting an immune response against HIV.

44. A method of eliciting an immune response in a subject susceptible to an HIV-related disease or condition comprising administering a composition of claim 40 to the subject, thereby eliciting an immune response against HIV.

45. A nucleic acid vector comprising at least one lentivirus sequence operably linked to a promoter, wherein the lentivirus sequence(s) encode a protein(s) that does not assemble into viral particles.

46. The nucleic acid vector of claim 45, wherein the lentivirus sequence comprises HIV, FIV, SIV, and EIAV sequences.

47. A nucleic acid vector comprising at least two lentivirus sequences, wherein the lentivirus sequences are each operably linked to separate promoters and wherein the HIV sequences encode proteins that do not assemble into viral particles.

48. The nucleic acid vector of claim 47, wherein the lentivirus sequences comprise HIV, FIV, SIV, and EIAV sequences.

49. A nucleic acid vector comprising at least two lentivirus sequences, wherein the lentivirus sequences:
a) Are each operably linked to separate promoters,
b) Encode proteins that do not assemble into viral particles, and
c) At least one lentivirus sequence is operably linked to a heterologous leader sequence, the vector optionally further comprising a downstream transcriptional terminator, an origin of replication and a selectable marker gene.

50. The nucleic acid vector of claim 49, wherein the lentivirus sequences comprise HIV, FIV, SIV, and EIAV sequences.

51. A nucleic acid vector, wherein the vector is a modified vaccinia Ankara (MVA) vector comprising:
a) At least two lentivirus sequences inserted into deletion site III of the MVA genome; and
b) At least one lentivirus sequence inserted into deletion site II of the MVA genome;
and wherein each lentivirus sequence is operably linked to a separate promoter and wherein the lentivirus sequences encode proteins that do not assemble into viral particles.

52. The nucleic acid vector of claim 51, wherein the lentivirus sequences comprise HIV, FIV, SIV, and EIAV sequences.


SEQUENCE LISTING (48 sequences)

SEQ ID NO: 1 - protein, 23 aa, Artificial Sequence
  Description of Artificial Sequence Synthetic peptide
MDAMKRGLCCVLLLCGAVFVSAR

SEQ ID NO: 2 - protein, 9 aa, Artificial Sequence
  Description of Artificial Sequence Synthetic peptide
AMGMLKDTI

SEQ ID NO: 3 - protein, 20 aa, Artificial Sequence
  Description of Artificial Sequence Synthetic peptide
VPVWKEAKTTLFCASDAKAY

SEQ ID NO: 4 - protein, 20 aa, Artificial Sequence
  Description of Artificial Sequence Synthetic peptide
RNVSSDGTYNETYNEIKNCS

SEQ ID NO: 5 - protein, 20 aa, Artificial Sequence
  Description of Artificial Sequence Synthetic peptide
TSNPPIPVGDIYKRWIILGL

SEQ ID NO: 6 - protein, 9 aa, Artificial Sequence
  Description of Artificial Sequence Synthetic peptide
AMQMLKDTI

SEQ ID NO: 7 - DNA, 2460 bp, Homo sapiens
atggatgcaatgaagagagggctctgctgtgtgctgctgctgtgtggagcagtcttcgttagcgccgccgagaacttgtgggtgaccgtgtactacggcgtgcccgtgtggaaggaggccaagaccaccctgttctgcgcctccgacgccaaggcctacgagaaggaggtgcacaacgtgtgggccacccacgcctgcgtgcccaccgaccccaacccccaggagatggtgttggagaacgtgaccgagaacttcaacatgtggaagaacgacatggtgaaccagatgcacgaggacgtcatcagcttgtgggaccagagcctgaagccctgcgtgaagttgacccccctgtgcgtgaccttggagtgcaggaacgtgagcagcaacggcacctacaacgagacctacaacgagatcaagaactgctccttcaacgccaccaccgtgttgagggacaggaagcagaccgtgtacgccctgttctacaggctggacatcgtgcccctgaacaagaagaactccagcgagaactccagcgagtactacaggttgatcaactgcaacacctccgccatcacccaggcctgccccaaggtgaccttcgaccccatccccatccactactgcacccccgccggctacgccatcctgaagtgcaacgacaagaccttcaacggcaccggcccctgccacaacgtgagcaccgtgcagtgcacccacggcatcaagcccgtggtgtccacccagctgctgttgaacggcagcctggccgagagggagatcatcatcaggtccgagaacctgaccaacaacgtgaagaccatcatcgtgcacctgaaccagtccgtggagatcgtgtgcaccaggcccaacaacaacaccaggaagagcatcaggatcggccccggccagaccttctacgccaccggcgacatcatcggcgacatcaggcaggcccactgcaacatcagcaaggacaagtggaaggagaccttgcagagggtgggcaagaagttggccgagcacttccccaacaaagccatcgagttcgcctcctcctccggcggcgacctggagatcaccacccacagcttcaactgcaggggcgagttcttctactgcaacacctccagcctgttcaacggcacctacatgcccaacggcaccgagggcaactccagctccatcatcaccatcccctgcaggatcaagcagatcatcaacatgtggcaggaggtgggccgcgccatgtacgccccccccatcgagggcaacatcacctgcaagtccaacatcaccggcctgctgttggtgcgcgacggcggcaaggagaccaacgacaccgagaccttcaggcccggcggcggcgacatgagggacaactggaggagcgagttgtacaagtacaaggtggtggagatcaagcccttgggcatcgcccccaccgccgccaagaggagggtggtggagagggagaagagggccgtgggcatcggcgccaccgtgcaggcccgccagctgctgagcggcatcgtgcagcagcagagcaacctgctgcgcgccatcgaggcccagcagcacctgctgcagctgaccgtgtggggcatcaagcagctgcagacccgcgtcctggccatcgagcgctacctgaaggaccagcagctgctgggcatctggggctgcagcggcaagctgatctgcaccaccgccgtgcactggaacagcagctggagcaaccgcagccaggaggagatctggaacaacatgacctggatgcagtgggaccgcgagatcagcaactacaccaacaccatctaccgcctgctggaggacagccagaaccagcaggagcgcaacgagaaggacctgctggccctggacaactggaagaacctgtggagctggttcgacatcaccaactggctgtggtacatccgcatcttcatcatgatcgtgggcggcctgatcggcctgcgcatcatcttcgccgtgctgagcatcgtgaaccgcgtgcgccagggctacagccccctgagcttccagaccctgacccccaaccccggcggccccgaccgcctgggccgcatcgaggaggagggcggcgagcaggacaagaaccgcagcatccgcctggtgaacggcttcctggccctggcctgggacgacctgcgcaacctgtgccgcttcagctaccacctgctgcgcgacctgctgctgatcgtggcccgcatcgtggagctgctgggccgccgcggctgggaggccctgcgctactggtggaacctgctgaagtactgggtgcaggagctgaagaacagcgccgtgagcctgctgaacgccaccgccatcgccgtggccgagggcaccgaccgcgtgatcgaggtggtgcagggcgcctaccgcgccatcctgcacatcccccgccgcatccgccagggcttcgaggccgccctgcagtaa

SEQ ID NO: 8 - protein, 839 aa, Homo sapiens
MDAMKRGLCCVLLLCGAVFVSAAENLWVTVYYGVPVWKEAKTTLFCASDAKAYEKEVHNVWATHACVPTDPNPQEMVLENVTENFNMWKNDMVNQMHEDVISLWDQSLKPCVKLTPLCVTLECRNVSSNGTYNETYNEIKNCSFNATTVLRDRKQTVYALFYRLDIVPLNKKNSSENSSEYYRLINCNTSAITQACPKVTFDPIPIHYCTPAGYAILKCNDKTFNGTGPCHNVSTVQCTHGIKPVVSTQLLLNGSLAEREIIIRSENLTNNVKTIIVHLNQSVEIVCTRPNNNTRKSIRIGPGQTFYATGDIIGDIRQAHCNISKDKWKETLQRVGKKLAEHFPNKTIEFASSSGGDLEITTHSFNCRGEFFYCNTSSLFNGTYMPNGTEGNSSSIITIPCRIKQIINMWQEVGRAMYAPPIEGNITCKSNITGLLLVRDGGKETNDTETFRPGGGDMDDNWRSELYKYKVVEIKPLGIAPTAAKRRVVEREKRAVGIGAVFLGFLGAAGSTMGAASITLTVQARQLLSGIVQQQSNLLRAIEAQQHLLQLTVWGIKQLQTRVLAIERYLKDQQLLGIWGCSGKLICTTAVHWNSSWSNRSQEEIWNNMTWMQWDREISNYTNTIYRLLEDSQNQQERNEKDLLALDNWKNLWSWFDITNWLWYIRIFIMIVGGLIGLRIIFAVLSIVNRVRQGYSPLSFQTLTPNPGGPDRLGRIEEEGGEQDKNRSIRLVNGFLALAWDDLRNLCRFSYHLLRDLLLIVARIVELLGRRGWEALRYWWNLLKYWVQELKNSAVSLLNATAIAVAEGTDRVIEVVQGAYRAILHIPRRIRQGFEAALQ

SEQ ID NO: 9 - DNA, 1545 bp, Homo sapiens
atggacgccatgaagcgcggcctgtgctgcgtgctgctgctgtgcggcgccgtgttcgtgagcgcccgcatgggcgcccgcgccagcatcctgcgcggcggcaagctggacaagtgggagaagatccgcctgcgccccggcggcaagaagcactacatgctgaagcacctggtgtgggccagccgcgagctggagcgcttcgccctgaaccccggcctgctggagaccagcgagggctgcaagcagatcatcaagcagctgcagcccgccctgcagaccggcaccgaggagctgcgcagcctgttcaacaccgtggccaccctgtactgcgtgcacgagggcatcgagatccgcgacaccaaggaggccctggacaagatcgaggaggagcagaacaagatccagcagaagacccagcaggccaagaaggccgacgagaaggtgagccagaactaccccatcgtgcagaacctgcagggccagatggtgcaccaggccatctcccccaggaccttgaacgcctgggtgaaggtgatcgaggagaaggccttcagccccgaggtgatccccatgttcaccgccttgtccgagggcgccaccccccaggacttgaacaccatgttgaacaccgtgggcggccaccaggccgccatgcagatgttgaaggacaccatcaacgaggaggccgccgagtgggacagggtgcaccccgtgcacgccggccccatcgcccccggccagatgagggagcccaggggcagcgacatcgccggcaccaccagcaccctgcagggccagatcgcctggatgaccagcaacccccccgtgcccgtgggcgagatctacaagaggtggatcatcctgggcttgaacaagatcgtgaggatgtacagccccgtgagcatcttggacatcaagcagggccccaaggagcccttcagggactacgtggaccgcttcttcaagaccttgagggccgagcaggccacccaggacgtgaagaactggatgaccgacaccttgttggtgcagaacgccaaccccgactgcaagaccatcttgagggccttgggccccggcgcctccttggaggagatgatgaccgcctgccagggcgtgggcggccccagccacaaggccagggtgttggccgaggccatgagccaggccaacggcaccatcctgatgcagaggagcaacttcaagggctccaagaggatcgtgaagtgcttcaactgcggcaaggagggccacatcgccaggaactgcagggcccccaggaagaagggctgctggaagtgcggcaaggagggccaccagatgaaggactgcaccgagaggcaggccaacttcttgggcaagatctggccctcccacaagggcaggcccggcaacttcctgcagagcaggcccgagcccaccgccccccccgccgagagcttcaggttcgaggagaccacccccgcccccaagcaggagcccaaggacagggagcccttgacctccctgaagtccctgttcggcagcgaccccttgtcccagtaa

SEQ ID NO: 10 - protein, 514 aa, Homo sapiens
MDAMKRGLCCVLLLCGAVFVSARMGARASILRGGKLDKWEKIRLRPGGKKHYMLKHLVWASRELERFALNPGLLETSEGCKQIIKQLQPALQTGTEELRSLFNTVATLYCVHEGIEIRDTKEALDKIEEEQNKIQQKTQQAKKADEKVSQNYPIVQNLQGQMVHQAISPRTLNAWVKVIEEKAFSPEVIPMFTALSEGATPQDLNTMLNTVGGHQAAMQMLKDTINEEAAEWDRVHPVHAGPIAPGQMREPRGSDIAGTTSTLQGQIAWMTSNPPVPVGEIYKRWIILGLNKIVRMYSPVSILDIKQGPKEPFRDYVDRFFKTLRAEQATQDVKNWMTDTLLVQNANPDCKTILRALGPGASLEEMMTACQGVGGPSHKARVLAEAMSQANGTILMQRSNFKGSKRIVKCFNCGKEGHIARNCRAPRKKGCWKCGKEGHQMKDCTERQANFLGKIWPSHKGRPGNFLQSRPEPTAPPAESFRFEETTPAPKQEPKDREPLTSLKSLFGSDPLSQ

SEQ ID NO: 11 - DNA, 2901 bp, Homo sapiens
atggacgccatgaagcgcggcctgtgctgcgtgctgctgctgtgcggcgccgtgttcgtgagcgcccgcccccagatcaccctgtggcagcgccccctggtgtccatccgcgtgggcggccagatcaaggaggccctgctggacgacaccgtgttggaggaggtgaacttgcccggcaagtggaagcccaagatgatcggcggcatcggcggcttcatcaaggtgaggcagtacgaccagatccccatcgagatctgcggcaagaaggccatcggcaccgtgttggtgggccccacccccgtgaacatcatcggcaggaacatgttgacccagctgggctgcaccctgaacttccccatcagccccatcgagaccatccccgtgaagctgaagcccggcatggacggcccccgcgtgaagcagtggcccctgaccgaggagaagatcaaggccctgaccgccatctgcgacgagatggagaaggagggcaagatcaccaagatcggccccgagaacccctacaacacccccgtgttcgccatcaagaagaaggacagcaccaagtggcgcaagctggtggacttccgcgagctgaacaagcgcacccaggacttctgggaggtgcagctgggcatcccccaccccgccggcctgaagaagaagaagtccgtgaccgtgctggacgtgggcgacgcctacttctccgtgcccctgtacgaggacttccgcaagtacaccgccttcaccatccccagcatcaacaacgagacccccggcatccgctaccagtaccacgtgctgccccagggctggaagggctcccccgccatcttccagtgcagcatgaccaagatcctggagcccttccgggcccagaaccccgagatcgtgatctaccagtacggcgacgacctgtacgtgggctccgacctggagatcggccagcaccgcgccaagatcgaggagttgcgcgagcacctgctgaagtggggcttcaccacccccgacaagaagcaccagaaggagccccccttcctgtggatgggctacgagctgcaccccgacaagtggaccgtgcagcccatccagctgcccgagaaggacagctggaccgtgaacgacatccagaagctggtgggcaagctgaactgggccagccagatctaccccggcatcaaggtgcgccagctgtgcaagctgctgcgcggcgccaaggccctgaccgacatcatccccctgaccgaggaggccgagctggagctggccgagaaccgcgagatcctgaaggagcccgtgcacggcgcctactacgacccctccaaggacctgatcgccgagatccagaagcagggccaggaccagtggacctaccagatctaccaggagcccttcaagaacctgaagaccggcaagtacgccaagatgcgcaccgcccacaccaacgacgtgaagcagctgaccgaggccgtgcagaagatctccatggagagcatcgtgatctggggcaagatccccaagttccgcctgcccatccagaaggagacctgggagacccgctggaccgcctactggcaggccacctggatccccgagtgggagttcgtgaacaccccccccctggtgaagctgtggtaccagctggagaaggaccccatcgccggcgtggagaccttctacgtggacggcgccgccaaccgcgagaccaagatgggcaaggccggctacgtgaccgaccgcggccgccagaagatcgtgtccctgaccgagaccaccaaccagaagaccgagctgcaggccatctgcctggccttgcaggactccggctccgaggtgaacatcgtgaccgactcccagtacgccctgggcatcatccaggcccagcccgacaagagcgagtccgagctggtgaaccagatcatcgagcagctgatcaagaaggagcgcgtgtacctgtcctgggtgcccgcccacaagggcatcggcggcaacgagcaggtggacaagctggtgagcaacggcatccgcaaggtgctgttcctggacggcatcgacaaggcccaggaggagcacgagaagtaccacagcaactggcgcgccatggccagcgacttcaacctgccccccatcgtggccaaggagatcgtggccagctgcgaccagtgccagctgaagggcgaggccatgcacggccaggtggactgcagccccggcatctggcagctggactgcacccacctggagggcaagatcatcctggtggccgtgcacgtggccagcggctacatcgaggccgaggtgatccccgccgagaccggccaggagaccgcctacttcatcctgaagctggccggccgctggcccgtgaagatcatccacaccgacaacggcagcaacttcaccagcgccgccgtgaaggccgcctgctggtgggccggcatccagcaggagttcggcatcccctacaacccccagagccagggcgtggtggagtccatgaacaaggagctgaagaagatcatcggccaggtgcgcgaccaggccgagcacctgaagaccgccgtgcagatggccgtgttcatccacaacttcaagcgcaagggcggcatcggcggctacagcgccggcgagcgcatcatcgacatcatcgccaccgacatccagaccaaggagctgcagaagcagatcatcaagatccagaacttccgcgtgtactaccgcgacagccgcgaccccatctggaagggccccgccaagctgctgtggaagggcgagggcgccgtggtgatccaggacaacagcgacatcaaggtggtgccccgccgcaaggccaagatcatcaaggactacggcaagcagatggccggcgccgactgcgtggccagccgccaggacgaggactag

SEQ ID NO: 12 - protein, 966 aa, Homo sapiens
MDAMKRGLCCVLLLCGAVFVSARPQITLWQRPLVSIRVGGQIKEALLDDTVLEEVNLPGKWKPKMIGGIGGFIKVRQYDQIPIEICGKKAIGTVLVGPTPVNIIGRNMLTQLGCTLNFPISPIETIPVKLKPGMDGPRVKQWPLTEEKIKALTAICDEMEKEGKITKIGPENPYNTPVFAIKKKDSTKWRKLVDFRELNKRTQDFWEVQLGIPHPAGLKKKKSVTVLDVGDAYFSVPLYEDFRKYTAFTIPSINNETPGIRYQYNVLPQGWKGSPAIFQCSMTKILEPFRAQNPEIVIYQYGDDLYVGSDLEIGQHRAKIEELREHLLKWGFTTPDKKHQKEPPFLWMGTELHPDKWTVQPIQLPEKDSWTVNDIQKLVGKLNWASQIYPGIKVRQLCKLLRGAKALTDIIPLTEEAELELAENREILKEPVHGAYYDPSKDLIAEIQKQGQDQWTYQIYQEPFKNLKTGKYAKMRTAHTNDVKQLTEAVQKISMESIVIQGKIPKFRLPIQKETWETRWTAYWQATWIPEWEFVNTPPLVKLWWQLEKDPIAGVETFYVDGAANRETKMGKAGYVTDRGRQKIVSLTETTNQKTELQAICLALQDSGSEVNIVTDSQYALGIIQAQPDKSESELVNQIIEQLIKKERVYLSTVPAHKGIGGNEQVDKLVSNGIRKVLFLDGIDKAQEEHEKYHSNWRAMASDFNLPPIVAKEIVASCDQCQLKGEAMHGQVDCSPGIWQLDCTHLEGKIILVAVHVASGYIEAEVIPAETGQETAYFILKLAGRWPVKIIHTDNGSNFTSAAVKAACWWAGIQQEFGIPYNPQSQGVVESMNKELKKIIGQVRDQAEHLKTAVQMAVFIHNFKRKGGIGGYSAGERIIDIIATDIQTKELQKQIIKIQNFRVYYRDSRDPIWKGPAKLLWKGEGAVVIQDNSDIKVVPRRKAKIIKDYGKQMAGADCVASRQDED

SEQ ID NO: 13 - DNA, 993 bp, Homo sapiens
atggacgccatgaagcgcggcctgtgctgcgtgctgctgctgtgcggcgccgtgttcgtgagcgcccgcatgggcggcaagtggtccaagagcagcatcgtgggctggcccgccatccgcgagcgcattcgccgcaccgagcccgccgccgacggcgtgggcgccgtgtctcgcgacctggagaagcatggcgccatcaccagtaacaacaccgccgacaccaacgccgactgcgcctggctggagacccaggaggaggaggaggtgggcttccccgtccgcccccaggtgcccttgcgccccatgaccttcaagggcgccttggacctcagcttcttcctgaaggagaagggcggcctggaggggttgatctacagcaagaagaggcaggagatcctggacttgtgggtctaccacacccagggctacttccccgactggcacaactacacccccggccccggcgtccgcttccccctgaccttcggctggtgcttcaagctggtgcccgtggaccccggcgaggtggaggaggccaacgagggcgagaacaactgcttgctgcaccccgtctgccagcacggcatggacgacgagcaccgcgaggtgctgaagtggaagttcgacagccagctggcccaccgccacagggcccgcgagctgcacccggagttctacaaggactgcatggagcccgtggaccccaacctggagccctggaaccaccccggcagccagcccgagaccgcctgcaacaactgctactgcaagcgctgcagctaccactgcctggtgtgcttccagaagaagggcctgggcatcagctacggccgcaagaagcgccgccagcgccgcagcgccccccccagcagcgaggaccaccagaaccccatcagcaagcagcccctgccccgcacccagggcgaccccaccggcagcgaggagagcaagaagaaggtggagagcaagaccaagaccgaccccttcgactag

SEQ ID NO: 14 - protein, 330 aa, Homo sapiens
MDAMKRGLCCVLLLCGAVFVSARMGGKWSKSSIVGWPAIRERIRRTEPAADGVGAVSRDLEKHGAITSNNTADTNADCAWLETQEEEEVGFPVRPQVPLRPMTFKGALDLSFFLKEKGGLEGLIYSKKRQEILDLWVYHTQGYFPDWHNYTPGPGVRFPLTFGWCFKLVPVDPGEVEEANEGENNCLLHPVCQHGMDDEHREVLKWKFDSQLAHRHRARELHPEFYKDCMEPVDPNLEPWNHPGSQPETACNNCYCKRCSYHCLVCFQKKGLGISYGRKKRRQRRSAPPSSEDHQNPISKQPLPRTQGDPTGSEESKKKVESKTKTDPFD

SEQ ID NO: 15 - DNA, 8186 bp, Homo sapiens
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccgccaccatggatgcaatgaagagggggctctgctgtgtgctgctgctgtgtggagcagtcttcgttagcgccgccgagaacttgtgggtgaccgtgtactacggcgtgcccgtgtggaaggaggccaagaccaccctgttctgcgcctccgacgccaaggcctacgagaaggaggtgcacaacgtgtgggccacccacgcctgcgtgcccaccgaccccaacccccaggagatggtgttggagaacgtgaccgagaacttcaacatgtggaagaacgacatggtgaaccagatgcacgaggacgtcatcagcttgtgggaccagagcctgaagccctgcgtgaagttgacccccctgtgcgtgaccttggagtgcaggaacgtgagcagcaacggcacctacaacgagacctacaacgagatcaagaactgctccttcaacgccaccaccgtgttgagggacaggaagcagaccgtgtacgccctgttctacaggctggacatcgtgcccctgaacaagaagaactccagcgagaactccagcgagtactacaggttgatcaactgcaacacctccgccatcacccaggcctgccccaaggtgaccttcgaccccatccccatccactactgcacccccgccggctacgccatcctgaagtgcaacgacaagaccttcaacggcaccggcccctgccacaacgtgagcaccgtgcagtgcacccacggcatcaagcccgtggtgtccacccagctgctgttgaacggcagcctggccgagagggagatcatcatcaggtccgagaacctgaccaacaacgtgaagaccatcatcgtgcacctgaaccagtccgtggagatcgtgtgcaccaggcccaacaacaacaccaggaagagcatcaggatcggccccggccagaccttctacgccaccggcgacatcatcggcgacatcaggcaggcccactgcaacatcagcaaggacaagtggaaggagaccttgcagagggtgggcaagaagttggccgagcacttccccaacaagaccatcgagttcgcctcctcctccggcggcgacctggagatcaccacccacagcttcaactgcaggggcgagttcttctactgcaacacctccagcctgttcaacggcacctacatgcccaacggcaccgagggcaactccagctccatcatcaccatcccctgcaggatcaagcagatcatcaacatgtggcaggaggtgggccgcgccatgtacgccccccccatcgagggcaacatcacctgcaagtccaacatcaccggcctgctgttggtgcgcgacggcggcaaggagaccaacgacaccgagaccttcaggcccggcggcggcgacatgagggacaactggaggagcgagttgtacaagtacaaggtggtggagatcaagcccttgggcatcgcccccaccgccgccaagaggagggtggtggagagggagaagagggccgtgggcatcggcgccgtgttcctgggcttcctgggcgccgccggcagcaccatgggcgccgccagcatcaccctgaccgtgcaggcccgccagctgctgagcggcatcgtgcagcagcagagcaacctgctgcgcgccatcgaggcccagcagcacctgctgcagctgaccgtgtggggcatcaagcagctgcagacccgcgttctggccatcgagcgctacctgaaggaccagcagctgctgggcatctggggctgcagcggcaagctgatctgcaccaccgccgtgcactggaacagcagctggagcaaccgcagccaggaggagatctggaacaacatgacctggatgcagtgggaccgcgagatcagcaactacaccaacaccatctaccgcctgctggaggacagccagaaccagcaggagcgcaacgagaaggacctgctggccctggacaactggaagaacctgtggagctggttcgacatcaccaactggctgtggtacatccgcatcttcatcatgatcgtgggcggcctgatcggcctgcgcatcatcttcgccgtgctgagcatcgtgaaccgcgtgcgccagggctacagccccctgagcttccagaccctgacccccaaccccggcggccccgaccgcctgggccgcatcgaggaggagggcggcgagcaggacaagaaccgcagcatccgcctggtgaacggcttcctggccctggcctgggacgacctgcgcaacctgtgccgcttcagctaccacctgctgcgcgacctgctgctgatcgtggcccgcatcgtggagctgctgggccgccgcggctgggaggccctgcgctactggtggaacctgctgaagtactgggtgcaggagctgaagaacagcgccgtgagcctgctgaacgccaccgccatcgccgtggccgagggcaccgaccgcgtgatcgaggtggtgcagggcgcctaccgcgccatcctgcacatcccccgccgcatccgccagggcttcgaggccgccctgcagtaagaattccgtgaggctccggtgcccgtcagtgggcagagcgcacatcgcccacagtccccgagaagttggggggaggggtcggcgattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcggccgccgccaccatggacgccatgaagcgcggcctgtgctgcgtgctgctgctgtgcggcgccgtgttcgtgagcgcccgcatgggcgcccgcgccagcatcctgcgcggcggcaagctgggcaagtgggagaagatccgcctgcgccccggcgacaagaagcactacatgctgaagcacctggtgtgggccagccgcgagctggagcgcttcgccctgaaccccggcctgctggagaccagcgagggctgcaagcagatcatcaagcagctgcagcccgccctgcagaccggcaccgaggagctgcgcagcctgttcaacaccgtggccaccctgtactgcgtgcacgagggcatcgagatccgcgacaccaaggaggccctggacaagatcgaggaggagcagaacaagatccagcagaagacccagcaggccaagaaggccgacgagaaggtgagccagaactaccccatcgtgcagaacctgcagggccagatggtgcaccaggccatctcccccaggaccttgaacgcctgggtgaaggtgatcgaggagaaggccttcagccccgaggtgatccccatgttcaccgccttgtccgagggcgccaccccccaggacttgaacaccatgttgaacaccgtgggcggccaccaggccgccatgcagatgttgaaggacaccatcaacgaggaggccgccgagtgggacagggtgcaccccgtgcacgccggccccattgcccccggccagatgagggagcccaggggcagcgacatcgccggcaccaccagcaccctgcagggccagatcgcctggatgaccagcaacccccccgtgcccgtgggcgagatctacaagaggtggatcatcctgggcttgaacaagatcgtgaggatgtacagccccgtgagcatcttggacatcaagcagggccccaaggagcccttcagggactacgtggaccgcttcttcaagaccttgagggccgagcaggccacccaggacgtgaagaactggatgaccgacaccttgttggtgcagaacgccaaccccgactgcaagaccatcttgagggccttgggccccggcgcctccttggaggagatgatgaccgcctgccagggcgtgggcggccccagccacaaggccagggtgttggccgaggccatgagccaggccaacggcaccatcctgatgcagaggagcaacttcaagggctccaagaggatcgtgaagtgcttcaactgcggcaaggagggccacatcgccaggaactgcagggcccccaggaagaagggctgctggaagtgcggcaaggagggccaccagatgaaggactgcaccgagaggcaggccaacttcttgggcaagatctggccctcccacaagggcaggcccggcaacttcctgcagagcaggcccgagcccaccgccccccccgccgagagcttcaggttcgaggagaccacccccgcccccaagcaggagcccaaggacagggagcccttgacctccctgaagtccctgttcggcagcgaccccttgtcccagtaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 16 - DNA, 8017 bp, Homo sapiens
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccaccatggacgccatgaagcgcggcctgtgctgcgtgctgctgctgtgcggcgccgtgttcgtgagcgcccgcccccagatcaccctgtggcagcgccccctggtgtccatccgcgtggggggccagatcaaggaggccctgctggacgacaccgtgttggaggaggtgaacttgcccggcaagtggaagcccaagatgatcggcggcatcggcggcttcatcaaggtgaggcagtacgaccagatccccatcgagatctgcggcaagaaggccatcggcaccgtgttggtgggccccacccccgtgaacatcatcggcaggaacatgttgacccagctgggctgcaccctgaacttccccatcagccccatcgagaccatccccgtgaagctgaagcccggcatggacggcccccgcgtgaagcagtggcccctgaccgaggagaagatcaaggccctgaccgccatctgcgacgagatggagaaggagggcaagatcaccaagatcggccccgagaacccctacaacacccccgtgttcgccatcaagaagaaggacagcaccaagtggcgcaagctggtggacttccgcgagctgaacaagcgcacccaggacttctgggaggtgcagctgggcatcccccaccccgccggcctgaagaagaagaagtccgtgaccgtgctggacgtgggcgacgcctacttctccgtgcccctgtacgaggacttccgcaagtacaccgccttcaccatccccagcatcaacaacgagacccccggcatccgctaccagtacaacgtgctgccccagggctggaagggctcccccgccatcttccagtgcagcatgaccaagatcctggagcccttccgggcccagaaccccgagatcgtgatctaccagtacggcgacgacctgtacgtgggctccgacctggagatcggccagcaccgcgccaagatcgaggagttgcgcgagcacctgctgaagtggggcttcaccacccccgacaagaagcaccagaaggagccccccttcctgtggatgggctacgagctgcaccccgacaagtggaccgtgcagcccatccagctgcccgagaaggacagctggaccgtgaacgacatccagaagctggtgggcaagctgaactgggccagccagatctaccccggcatcaaggtgcgccagctgtgcaagctgctgcgcggcgccaaggccctgaccgacatcatccccctgaccgaggaggccgagctggagctggccgagaaccgcgagatcctgaaggagcccgtgcacggcgcctactacgacccctccaaggacctgatcgccgagatccagaagcagggccaggaccagtggacctaccagatctaccaggagcccttcaagaacctgaagaccggcaagtacgccaagatgcgcaccgcccacaccaacgacgtgaagcagctgaccgaggccgtgcagaagatctccatggagagcatcgtgatctggggcaagatccccaagttccgcctgcccatccagaaggagacctgggagacccgctggaccgcctactggcaggccacctggatccccgagtgggagttcgtgaacaccccccccctggtgaagctgtggtaccagctggagaaggaccccatcgccggcgtggagaccttctacgtggacggcgccgccaaccgcgagaccaagatgggcaaggccggctacgtgaccgaccgcggccgccagaagatcgtgtccctgaccgagaccaccaaccagaagaccgagctgcaggccatctgcctggccttgcaggactccggctccgaggtgaacatcgtgaccgactcccagtacgccctgggcatcatccaggcccagcccgacaagagcgagtccgagctggtgaaccagatcatcgagcagctgatcaagaaggagcgcgtgtacctgtcctgggtgcccgcccacaagggcatcggcggcaacgagcaggtggacaagctggtgagcaacggcatccgcaaggtgctgttcctggacggcatcgacaaggcccaggaggagcacgagaagtaccacagcaactggcgcgccatggccagcgacttcaacctgccccccatcgtggccaaggagatcgtggccagctgcgaccagtgccagctgaagggcgaggccatgcacggccaggtggactgcagccccggcatctggcagctggactgcacccacctggagggcaagatcatcctggtggccgtgcacgtggccagcggctacatcgaggccgaggtgatccccgccgagaccggccaggagaccgcctacttcatcctgaagctggccggccgctggcccgtgaagatcatccacaccgacaacggcagcaacttcaccagcgccgccgtgaaggccgcctgctggtgggccggcatccagcaggagttcggcatcccctacaacccccagagccagggcgtggtggagtccatgaacaaggagctgaagaagatcatcggccaggtgcgcgaccaggccgagcacctgaagaccgccgtgcagatggccgtgttcatccacaacttcaagcgcaagggcggcatcggcggctacagcgccggcgagcgcatcatcgacatcatcgccaccgacatccagaccaaggagctgcagaagcagatcatcaagatccagaacttccgcgtgtactaccgcgacagccgcgaccccatctggaagggccccgccaagctgctgtggaagggcgagggcgccgtggtgatccaggacaacagcgacatcaaggtggtgccccgccgcaaggccaagatcatcaaggactacggcaagcagatggccggcgccgactgcgtggccagccgccaggacgaggactaggaattccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcggccgccgccaccatggacgccatgaagcgcggcctgtgctgcgtgctgctgctgtgcggcgccgtgttcgtgagcgcccgcatgggcggcaagtggtccaagagcagcatcgtaggctggcccgccatccgcgagcgcatccgccgcaccgagcccgccgccgacggcgtgggcgccgtgtctcgcgacctggagaagcatggcgccatcaccagtaacaacaccgccgacaccaacgccgactgcgcctggctggagacccaggaggaggaggaggtgggcttccccgtccgcccccaggtgcccttgcgccccatgaccttcaagggcgccttggacctcagcttcttcctgaaggagaagggcggcctggaggggttgatctacagccagaagaggcaggagatcctggacttgtgggtctaccacacccagggctacttccccgactggcacaactacacccccggccccggcgtccgcttccccctgaccttcggctggtgcttcaagctggtgcccgtggaccccggcgaggtggaggaggccaacgagggcgagaacaactgcttgctgcaccccgtctgccagcacggcatggacgacgagcaccgcgaggtgctgaagtggaagttcgacagccagctggcccaccgccacagggcccgcgagctgcacccggagttctacaaggactgcatggagcccgtggaccccaacctggagccctggaaccaccccggcagccagcccgagaccgcctgcaacaactgctactacaagcgctgcagctaccactgcctggtgtgcttccagaagaagggcctgggcatcagctacggccgcaagaagcgccgccagcgccgtagcgccccccccagcagcgaggaccaccagaaccccatcagcaagcagcccctgccccgcacccagggcgaccccaccggcagcgaggagagcaagaagaaggtggagagcaagaccaagaccgaccccttcgactagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcccacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 17 - DNA, 2442 bp, Homo sapiens
atgagagtgacggggatcaggaagaactatcagcatttatggagatggggcaccatgctccttgggatgttgatgatctgtagtgctgcagaaaacttgtgggtcacagtctattatggggtacctgtgtggaaagaagccaaaactactctattctgtgcgtcagatgctaaagcatatgagaaagaagtgcataatgtctgggctacacatgcctgtgtacccacagaccccaacccacaagaaatggttttggaaaatgtaacagaaaattttaacatgtggaaaaatgacatggtgaatcagatgcatgaggatgtaatcagtttatgggatcaaagcctaaagccatgtgtaaagttgaccccactctgtgtcactttagaatgtagaaatgttagcagtaatggtacctacaatgagacctacaatgagatcaaaaattgctctttcggggccgggtattatagattaataaattgtaatacctcagccataacacaagcctgtccaaaggtcacttttgatccaattcctatacactattgcactccagctggttatgcgattctaaagtgtaatgataagacattcaatggaacaggaccatgccataatgttagtacagtacaatgtacacatggaattaagccagtggtatcaactcaactactgttaaatggtagcctagcagaaagagagataataattagatctgaaaatctgacaaacaatgtcaaaacaataatagtacatcttaatcaatctgtagaaattgtatgtacaagacccaacaataatacaagaaaaagtataaggataggaccaggacaaacattctatgcaacaggagacataataggagatataagacaagcacattgtaacattagtaaagataaatggaatgaaactttacaaagggtaggtaaaaaattagcagaacacttccctaataaaacaatagaatttgcatcatcctcaggaggggacctagaaattacaacacatagctttaattgtagaggagaatttttctattgtaatacatcaatcctgtttaatggtacatacatgcctaatggtacagaaggtaattcaagctcaatcatcacaatcccatgcagaataaagcaaattataaacatgtggcaggaggtaggacgagcaatgtatgcccctcccattgagggaaacataacatgtaaatcaaatatcacaggactactattggtacgtgatggaggaaaagagacaaatgatacagagacattcagacctggaggaggagatatgagggacaattggagaagtgaattatataaatataaagtggtagaaattaagccattgggaatagcacccactgcagcaaaaaggagagtggtggagagagaaaaaagagcagtgggaataggagctgtgttccttgggttcttgggagcagcaggaagcactatgggcgcggcgtcaataacgctgacggtacagtccagacaattgttgtctggtatagtgcaacagcaaagcaatttgctgagggctatagaggcgcaacagcatctgttgcaactcacggtctggggcattaagcagctccagacaagagtcctggctatagaaagatacctaaaggatcaacagctcctagggatttggggctgctctggaaaactcatctgcactactgctgtacattggaactccagttggagtaacagatctcaagaagagatttggaataacatgacttggatgcagtgggatagagaaattagtaattacacaaacacaatatacaggttgcttgaagactcgcaaaaccagcaggaaagaaatgaaaaagatttactagcattggacaattggaaaaatctatggagttggtttgacataacaaattggctgtggtatataagaatattcataatgatagtaggaggcttgataggtttaagaataatttttgctgtgctctctatagtgaatagagttaggcagggatactcacctttgtcgtttcagacccttaccccgaacccagggggacccgacaggctcggaagaatcgaagaagaaggtggagagcaagacaaaaacagatccattcgattagtgaacggattcttagcacttgcctgggacgacctgcggaacctgtgccgcttcagctaccacctcttgagagacttactcttgattgtagcaaggattgtggaacttctgggacgcagggggtgggaagccctcagatattggtggaatctcctgaagtattgggttcaggaactaaagaatagtgctgttagtttgctcaatgccacagctatagcagtagctgaggggacagatagggttatagaagtagtacaaggagcttatagagctattctccacatacctagaagaataagacagggctttgaagcagctttgcaataa

SEQ ID NO: 18 - protein, 813 aa, Homo sapiens
MRVTGIRKNYQHLWRWGTMLLGMLMICSAAENLWVTVYYGVPVWKEAKTTLFCASDAKAYEKEVHNVWATHACVPTDPNPQEMVLENVTENFNMWKNDMVNQMHEDVISLWDQSLKPCVKLTPLCVTLECRNVSSNGTYNETYNEIKNCSFGAGYYRLINCNTSAITQACPKVTFDPIPIHYCTPAGYAILKCNDKTFDGTGPCHNVSTVQCTHGIKPVVSTQLLLNGSLAEREIIIRSENLTNNVKTIIVHLNQSVEIVCTRPNNNTRKSIRIGPGQTFYATGDIIGDIRQAHCNISKDKWNETLQRVGKKLAEHFPNKTIEFASSSGGDLEITTHSFNCRGEFFYCNTSILFNGTYMPNGTEGNSSSIITIPCRIKQIINMWQEVGRAMYAPPIEGNITCKSNITGLLLVRDGGKETNDTETFRPGGGDMRDNWRSELYKYKVVEIKPLGIAPTAAKRRVVEREKRAVGIGAVFLGFLGAAGSTMGAASITLTVQSRQLLSGIVQQQSNLLRAIEAQQHLLQLTVWGIKQLQTRVLAIERYLKDQQLLGIWGCSGKLICTTAVHWNSSWSNRSQEEIWNNMTWMQWDREISNYTNTIYRLLEDSQNQQERNEKDLLALDNWKNLWSWFDITNWLWYIRIRIMIVGGLIGLRIIFAVLSIVNRVRQGYSPLSFQTLTPNPGGPDRLGRIQEEGGEQDKNRSIRLVNGFLALAWDDLRNLCRFSYHLLRDLLLIVARIVELLGRRGWEALRYWWNLLKYWVQELKNSAVSLLNATAIAVAEGTDRVIEVVQGAYRAILHIPRRIRQGFEAALQ

SEQ ID NO: 19 - DNA, 4404 bp, Homo sapiens
gccgccaccatggacgccatgaagcgcggcctgtgctgcgtgctgctgctgtgcggcgccgtgttcgtgagcgcccgcatgggtgcgagagcgtcaatattaagagggggaaaattagataaatgggaaaaaattaggttaaggccagggggaaagaaacactatatgctaaaacacatagtatgggcaagcagagagctggaaagatttgcacttaaccctggccttttagagacatcagaaggctgtaaacaaataataaaacagctacaaccagctcttcagacaggaacagaggaacttagatcattattcaacacagtagcaactctctattgtgtacatgcagggatagaagtacgagacaccaaagaagccttagacaagatagaggaagaacaaaacaaaatgcagcaaaaaacacagcaggcaaaagaggctgacgggaaggtcagtcaaaattatcctatagtgcagaatctccaagggcaaatggtgcaccaggccatatcacctagaactttgaatgcatgggtaaaagtaatagaggaaaaggcttttagcccagaggtaatacccatgtttacagcattatcagaaggagccaccccacaagatttaaacaccatgttaaatacagtagggggacatcaggcagccatgcaaatgttaaaagatactatcaatgaagaggctgcagaatgggatagagtacatccagtacatgcagggcctattgcaccaggccaaatgagagaaccaaggggaagtgacatagcaggaactactagtacccttcagggacaaatagcatggatgacgagtaacccacctgttccagtgggagaaatctataaaagatggataattctggggttaaataaaatagtaagaatgtatagccctgtgagcattttggacataaaacaagggccaaaggaaccctttagagactatgtagaccggttctttaaaactttaagagctgaacaagctacacaagatgtaaaaaattggatgacagacaccttgttggtccaaaatgcgaacccagattgtaagaccattttaagagcattaggaccaggggcttcattagaagagatgatgacagcatgtcagggagtgggaggacctaaccacaaagcaagagtgttggctgaggcaatgagccaagcaaacggtaccatactgatgcagagaagcaattttaaaggctctaaaagaattgttaaatgtttcaactgtggcaaggaagggcacatagccagaaattgcagggcccctaggaaaaaaggctgttggaaatgtggaaaggaaggacaccaaatgaaagactgcactgaaaggcaggctaattttttagggaaaatttggcctccccacaaggggaggccagggaatttcctccagagcagaccagagccaacagccccaccagcagagagcttcgggttcgaggagacaaccccagctccgaagcaggagccgaaagacagggaacccttaacttccctcaaatcactctttggcagcgaccccttgtctcaacctcaaatcactctttggcagcgaccccttgtctcaataagagtagggggccagataaaagaggctctcttagatgatacagtattagaagaagtaaatttgccaggaaaatggaaaccaaaaatgataggaggaattggaggttttatcaaagtaagacaatatgatcaaatacctatagaaatttgtggaaaaaaggctataggtacagtattagtgggacccacacctgtcaacataattggaagaaatatgttgactcaacttggatgcacactaaattttccaatcagtcccattgaaactataccagtaaaattaaagccaggaatggatggcccaagggttaaacaatggccattgacagaagagaaaataaaagcattaacagcaatttgtgatgaaatggagaaggaaggaaaaattacaaaaattgggcctgaaaatccatataacactccagtatttgccataaaaaagaaggacagtactaagtggagaaaattagtagatttcagggaactcaataaaagaactcaagatttttgggaagttcaattaggaataccacacccagcagggttaaaaaagaaaaaatcagtgacagtactggatgtgggggatgcatatttttcagttcctttatatgaagacttcagaaaatatactgcattcaccatacctagtataaacaatgaaacaccagggatcaggtatcaatataatgtgcttccacagggatggaagggatcaccagcaatattccagtgtagcatggcaaaaatcttagagccctttagggcacaaaatccagaaatagtcatctatcaatatggcgatgacttgtatgtaggatctgacttagagatagggcaacatagagcaaaaatagaggagttaagagaacatctgttaaagtggggatttaccacaccagacaagaaacatcagaaagaacctccatttctttggatggggtatgaactccatcctgacaaatggacagtacagcctatacagctgccagaaaaggatagctggactgtcaatgatatacagaagttagtgggaaaattaaactgggcaagtcagatttacccaggaattaaagtaaggcaactttgtaaactccttaggggggccaaagcactaacagacataataccactaactgaagaagcagaattggagttggcagaaaacagggaaattctaaaagaaccagtacatggagcatattatgacccatcaaaagacttgatagctgaaatacagaaacaggggcaggaccaatggacatatcaaatttaccaagaaccattcaaaaatctgaaaacagggaaatatgcaaaaatgaggactgcccacactaatgatgtaaaacagttaacagaggctgtgcagaaaatatccatggaaagcatagtaatatggggaaaaattcctaaatttaggttacccatcccaaaagaaacctgggagacacggtggacagcctattggcaagccacctggattcctgagtgggaatttgttaatacccctcccttagtaaaattatggtaccagctggagaaagatcccatagcaggagtagaaactttctatgtagatggagcagctaatagggaaattaaaatgggaaaagcagggtatgttactgacagaggaaggcagaaaattgtgtctctaactgaaacaacaaatcagaagactgaattgcaagcaatttgtctagctttgcaagattcaggatcagaagtaaatatagtaacagattcacagtatgcattaggaatcattcaagcacaaccagataagagtgagtcagagttagttaaccaaataatagaacaattaataaaaaaggaaagggtctacctgtcgtgggtaccagcacataaaggaattggaggaaatgaacaagtagataaattagtaagtaatggaatcaggaaagtgctatttctagatggaatagataaagctcaagaagagcatgaaaagtatcacagcaattggagagcaatggctagtgactttaatctgccacccatagtagcaaaagaaatagtagctagctgtgatcaatgtcagctaaaaggggaagccatgcatggacaagtagactgtagtccagggatatggcaattagattgtacacatttagaaggaaaaatcatcctggtagcagtccatgtagccagtggctacatagaagcagaagttatcccagcagaaacaggacaagaaacagcatactttatactaaaattagcaggaagatggccagtcaaaataatacatacagacaatggtagcaatttcaccagtgctgcagttaaggcagcctgttggtgggcaggtatccaacaggaatttggaattccctacaatccccaaagtcagggagtagtagaatccatgaataaagaattaaagaaaattatagggcaggtaagagatcaagctgagcaccttaagacagcagtacaaatggcagtattcattcacaattttaaaagaaaaggggggattggggggtacagtgcaggggaaagaataatagacataatagcaacagacatacaaactaaagaattacaaaaacaaattataaagattcaaaattttcgggtttattacagagacagcagagaccccatttggaaaggaccagccaaactactctggaaaggtgaaggggcagtagtaatacaagataatagtgacataaaggtagtaccaaggaggaaagcaaaaatcattaagggctatggaaaacagatggcaggtgctgattgtgtggcaagtagacaggatgaagattagtaattttttatgcggccgcta

SEQ ID NO: 20 - protein, 1457 aa, Homo sapiens
MDAMKRGLCCVLLLCGAVFVSARMGARASILRGGKLDKWEKIRLRPGGKKHYMLKHIVWASRELERFALNPGLLETSEGCKQIIKQLQPALQTGTEELRSLFNTVATLYCVHAGIEVRDTKEALDKIEEEQNKMQQKTQQAKEADGKVSQNYPIVQNLQGQMVHQAISPRTLNAWVKVIEEKAFSPEVIPMFTALSEGATPQDLNTMLNTVGGHQAAMQMLKDTINEEAAEWDRVHPVHAGPIAPGQMREPRGSDIAGTTSTLQGQIAWMTSNPPVPVGEIYKRWIILGLNKIVRMYSPVSILDIKQGPKEPFRDYVDRFFKTLRAEQATQDVKNWMTDTLLVQNANPDCKTILRALGPGASLEEMMTACQGVGGPNHKARVLAEAMSQANGTILMQRSNFKGSKRIVKCFNCGKEGHIARNCRAPRKKGCWKCGKEGHQMKDCTERQANFLGKIWPPHKGRPGNFLQSRPEPTAPPAESFGFEETTPAPLQEPKDREPLTSLKSLFGSDPLSQPQITLWQRPLVSIRVGGQIKEALLDDTVLEEVNLPGKWKPKMIGGIGGFIKVRQYDQIPIEICGKKAIGTVLVGPTPVNIIGRNMLTQLGCTLNFPISPIETIPVKLKPGMDGPRVKQWPLTEEKIKALTAICDEMEKEGKITKIGPENPYNTPVFAIKKKDSTKWRKLVDFRELNKRTQDFWEVQLGIPHPAGLKKKKSVTVLDVGDAYFSVPLYEDFRKYTAFTIPSINNETPGIRYQYNVLPQGWKGSPAIFQCSMAKILEPFRAQNPEIVIYQYGDDLYVGSDLEIGQHRAKIEELREHLLKWGFTTPDKKHQKEPPFLWMGYELHPDKWTVQPIQLPEKDSWTVNDIQKLVGKLNWASQIYPGIKVRQLCKLLRGAKALTDIIPLTEEAELELAENREILKEPVHGAYYDPSKDLIAEIQKQGQDQWTYQIYQEPFKNLKTGKYAKMRTAHTNDVKQLTEAVQKISMESIVIWGKIPKFRLPIPKETWETRWTAYWQATWIPEWEFVNTPPLVKLWYQLEKDPIAGVETFYVDGAANRETKMGKAGYVTDRGRQKIVSLTETTNQKTELQAICLALQDSGSEVNIVTDSQYALGIIQAQPDKSESELVNQIIEQLIKKERVYLSWVPAHKGIGGNEQVDKLVSNGIRKVLFLDGIDKAQEEHEKYHSNWRAMASDFNLPPIVAKEIVASCDQCQLKGEAMHGQVDCSPGIWQLDCTHLQGKIILVAVHVASGYIEAEVIPAETGQETAYFILKLAGRWPVKIIHTDNGSNFTSAAVKAACWWAGIQQEFGIPYNPQSQGVVESMNKELKKIIGQVRDQAEHLKTAVQMAVFIHNFKRKGGIGGYSAGERIIDIIATDIQTKELQKQIIKIQNFRVYYRDSRDPIWKGPAKLLWKGEGAVVIQDNSDIKVVFRRKAKIIKGYGKQMAGADCVASRQDED

SEQ ID NO: 21 - DNA, 1011 bp, Homo sapiens
gccgccaccatggacgccatgaagcgcggcctgtgctgcgtgctgctgctgtgcggcgccgtgttcgtgagcgcccgcatggggggcaagtggtcaaaaagtagcatagttggatggcctgctataagagaaagaataagacgaactgaaccagcagcagatggggtgggagcagtatctcgagacctggaaaaacatggagcaatcacaagtaacaacacagcagatactaatgctgattgtgcctggctagaaacacaagaggaggaggaggtgggttttccagtcagacctcaggtacccttaagaccaatgacttttaagggagcattggatctcagcttctttttaaaagaaaaggggggactggaagggttaatttactctaagaaaaggcaagagatccttgatttgtgggtctatcacacacaaggctacttccctgactggcacaactacacaccaggaccaggggtcagattcccactgacttttgggtggtgcttcaagctagtaccagttgacccaggggaagtggaagaggccaatgaaggagaaaacaactgtttgctacaccctgtctgccagcatggaatggatgatgaacacagagaagtattaaagtggaagtttgacagtcaactagcacacagacacagggcccgcgagctacatccggagttttacaaagactgcatggagccagtagatcctaacctagagccctggaaccatccaggaagtcagcctgaaactgcttgcaataactgttattgtaaacgctgtagctaccattgtctagtttgctttcagaaaaaaggcttaggcatttcctatggcaggaagaagcggagacagcgacgaagcgctcctccaagcagtgaggatcatcaaaatcctatatcaaagcagcccttaccccgaacccagggggacccgacaggctcggaagaatcgaagaagaaggtggagagcaagacaaaaacagatccattcgattagtaatttttt

SEQ ID NO: 22 - protein, 330 aa, Homo sapiens; identical to SEQ ID NO: 14

SEQ ID NO: 23 - DNA, 9181 bp, Human immunodeficiency virus 1
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttc

SEQ ID NO: 24 - DNA, 1503 bp, Human immunodeficiency virus 1
  CDS (1)..(1500)
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataa

SEQ ID NO: 25 - protein, 500 aa, Human immunodeficiency virus 1
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 26 - DNA, 3012 bp, Human immunodeficiency virus 1
  CDS (1)..(3009)
ttttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattag

SEQ ID NO: 27 - protein, 1003 aa, Human immunodeficiency virus 1
FFREDLAFLQGKAREFSSEQTRANSPTRRELQVWGRDNNSPSEAGADRQGTVSFNFPQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 28 - DNA, 579 bp, Human immunodeficiency virus 1
  CDS (1)..(576)
atggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactag

SEQ ID NO: 29 - protein, 192 aa, Human immunodeficiency virus 1
MENRWQVMIVWQVDRMRIRTWKSLVKHHMYVSGKARGWFYRHHYESPHPRISSEVHIPLGDARLVITTYWGLHTGERDWHLGQGVSIEWRKKRYSTQVDPELADQLIHLYYFDCFSDSAIRKALLGHIVSPRCEYQAGHNKVGSLQYLALAALITPKKIKPPLPSVTKLTEDRWNKPQKTKGHRGSHTMNGH

SEQ ID NO: 30 - DNA, 237 bp, Human immunodeficiency virus 1
  CDS (1)..(234)
atggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgacatag

SEQ ID NO: 31 - protein, 78 aa, Human immunodeficiency virus 1
MEQAPEDQGPQREPHNEWTLELLEELKNEAVRHFPRIWLHGLGQHIYETYGDTWAGVEAIIRILQQLLFIHFQNWVST

SEQ ID NO: 32 - DNA, 261 bp, Human immunodeficiency virus 1
  CDS (1)..(258)
atggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcaacccacctcccaaccccgaggggacccgacaggcccgaaggaatag

SEQ ID NO: 33 - protein, 86 aa, Human immunodeficiency virus 1
MEPVDPRLEPWKHPGSQPKTACTNCYCKKCCFHCQVCFITKALGISYGRKKRRQRRRAHQNSQTHQASLSKQPTSQPRGDPTGPKE

SEQ ID NO: 34 - DNA, 351 bp, Human immunodeficiency virus 1
  CDS (1)..(348)
atggcaggaagaagcggagacagcgacgaagagctcatcagaacagtcagactcatcaagcttctctatcaaagcaacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatag

SEQ ID NO: 35 - protein, 116 aa, Human immunodeficiency virus 1
MAGRSGDSDEELIRTVRLIKLLYQSNPPPNPEGTRQARRNRRRRWRERQRQIHSISERILGTYLGRSAEPVPLQLPPLERLTLDCNEDCGTSGTQGVGSPQILVESPTVLESGTKE

SEQ ID NO: 36 - DNA, 249 bp, Human immunodeficiency virus 1
  CDS (1)..(246)
atgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtag

SEQ ID NO: 37 - protein, 82 aa, Human immunodeficiency virus 1
MQPIPIVAIVALVVAIIIAIVVWSIVIIEYRKILRQRKIDRLIDRLIERAEDSGNESEGEISALVEMGVEMGHHAPWDVDDL

SEQ ID NO: 38 - DNA, 2571 bp, Human immunodeficiency virus 1
  CDS (1)..(2568)
atgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataa

SEQ ID NO: 39 - protein, 856 aa, Human immunodeficiency virus 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 40 - DNA, 372 bp, Human immunodeficiency virus 1
  CDS (1)..(369)
atgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattag

SEQ ID NO: 41 - protein, 123 aa, Human immunodeficiency virus 1
MGGKWSKSSVIGWPTVRERMRRAEPAADRVGAASRDLEKHGAITSSNTAATNAACAWLEAQEEEEVGFPVTPQVPLRPMTYKAAVDLSHFLKEKGGLEGLIHSQRRQDILDLWIYHTQGYFPD

SEQ ID NO: 42 - protein, 4 aa, Human immunodeficiency virus 1
DTGA

SEQ ID NO: 43 - protein, 4 aa, Human immunodeficiency virus 1
YMDD

SEQ ID NO: 44 - protein, 4 aa, Human immunodeficiency virus 1
YGDD

SEQ ID NO: 45 - protein, 9 aa, Human immunodeficiency virus 1
TYNETYNEI

SEQ ID NO: 46 - protein, 15 aa, Human immunodeficiency virus 1
TAVQMAVFIHNFKRK

SEQ ID NO: 47 - protein, 15 aa, Human immunodeficiency virus 1
VHGVYYDPSKDLIAE

SEQ ID NO: 48 - protein, 15 aa, Human immunodeficiency virus 1
ISYGRKKRRQRRRAP